(19) United States
(12) Patent Application Publication
(10) Pub. No.: US 2021/0366586 A1
(43) Pub. Date: Nov. 25, 2021

(54) Enterprise Consumer Safety System

(71) Applicant: Kelly Dell Tyler, Rincón, PR (US)

(72) Inventors: Thomas Michael Ryan, Coeur d'Alene, ID (US); Thomas Russell Bartz, Leavenworth, WA (US); Richard Martin Homan, New York, NY (US); William Harrison Parker, Coeur d?Alene, ID (US); Kelly Dell Tyler, Rincón (PR)

(73) Assignee: Kelly Dell Tyler, Rincón, PR (US)

(21) Appl. No.: 17/257,503

(22) PCT Filed: Jul. 2, 2019
(86) PCT No.: PCT/US19/40423

§ 371 Date: Dec. 31, 2020

Related U.S. Application Data
(60) Provisional application No. 62/832,350, filed on Apr. 11, 2019.
(60) Provisional application No. 62/800,179, filed on Feb. 1, 2019.
(60) Provisional application No. 62/692,860, filed on Jul. 2, 2018.
(60) Provisional application No. 62/845,249, filed on May 8, 2019.

(51) Int. Cl. G16H 20/10; G06Q 20/20; G16H 10/60; G16H 50/20; G06Q 20/32; G16H 40/67; G06Q 20/38; G16H 70/40; G16H 15/00; G16H 40/20; G06F 16/9535

(52) U.S. Cl. CPC G16H 20/10; G06Q 30/014; G06Q 20/203; G16H 10/60; G16H 50/20; G06Q 20/204; G06Q 20/3224; G16H 40/67; G06Q 20/3278; G06Q 20/208; G06Q 20/389; G16H 70/40; G16H 15/00; G16H 40/20; G06F 16/9535; G06Q 20/202

(57) ABSTRACT

A data management system is disclosed wherein at least a portion of the data is generated at a point of sale (POS) and/or retail store server and the data is stored, at least in part, on a blockchain database or other secure database. A digital replica of a point of sale device is configured to receive a product unique identifier, and to receive customer unique identifying data. An adapter is configured to facilitate communication between incompatible entities. A repository is configured to store the product unique identifier and to store the customer unique identifying data associated with a purchase of the product. A query module configured to execute a query of the repository to identify customer unique identifying data for the customers who purchased the recalled product. A communication module is configured to receive a communication regarding a product recall and to send communications regarding a product recall.

This application claims the benefit of the filing date of U.S. Provisional Patent Application No. 62/692,860 filed Jul. 2, 2018, U.S. Provisional Patent Application No. 62/800,179 filed Feb. 1, 2019, U.S. Provisional Patent Application No. 62/832,350 filed Apr. 11, 2019, and U.S. Provisional Patent Application No. 62/845,249 filed May 8, 2019 the disclosures of which are hereby incorporated by reference herein in their entirety.

### FIELD

This disclosure relates generally to data systems and, more particularly, to a data management system wherein at least a portion of the data is generated at a point of sale (POS) and/or retail store server and the data is stored, at least in part, on a blockchain database or other secure database.

### BACKGROUND

Although there may be many techniques taught in the prior art for integrating a point of sale (POS) into services none are structured similarly to the present disclosure for the purpose of interacting with a consumer and/or other entities, and none offer all the advantages provided in the present disclosure.

The point of sale (POS) is the point at which a customer makes a payment to the merchant (retailer) in exchange for goods or services. It is the time and place (and/or device) where a transaction is completed. At the point of sale, the merchant (retailer) calculates the amount owed by a customer, specifies that amount, may prepare a receipt or invoice for the customer, and may specify payment options for the customer to make payment. After receiving payment, the merchant may or may not issue a receipt for the transaction, which may be printed or sent electronically.

The point of sale is often referred to as the point of service because it is not just a point of sale but the place where a merchant (retailer) interacts with the customer. With every customer in world using a point of sale to make purchases, it becomes increasingly important to provide additional services at the point of sale for customers, merchants and for the companies (brands) that make the products the customers purchase.

Thus, there is a technological need to provide extended services via the point of sale.

Each year there are hundreds of recalls for food, pharmaceuticals, and other products that have been deemed unsafe. Currently, only two recall notifications are required by federal law: a posting on the Food and Drug Administration's (FDA) recall website, and a press release issued by the company conducting the recall. Consumer advocates argue the current standard does not adequately inform consumers but instead creates an uneven system of recall notifications that vary from retailer to retailer. Today, what often happens when a product is recalled is the retailer prints a recall notice and posts this notice somewhere in the retail location that sold the recalled product. The recall notice, that is printed and posted, is seldom seen by most consumers who actually purchased the recalled product. Although retailers are typically not at fault for product contamination, they are tasked with a multitude of responsibilities, from removing product from their shelves to reassuring consumers to setting the record straight with news media, once a recall occurs. Industry has no consistent, effective way of notifying all (or most) end users of a recalled product. Social media efforts have been implemented by many retailers as a way to quickly reach consumers. For example, Facebook and Twitter can spread the word of a recall as users share and retweet news. This is a big change from the '80s and '90s when retailers relied on newspapers and television stations to inform shoppers. However, using social media, consumers can become unnecessarily alarmed or anxious about the quality and safety of products. Despite its speed, social media does present unique challenges. Users can easily spread false information. If a user believes he or she is a victim of contaminated food and tweets or posts about it, that post could go viral and create a panic that the retailer must fight to contain, requiring many retailers to employ social media specialists. Retailers have had to develop internal practices to make sure they are moving quickly to remove products from shelves and storage areas while at the same time communicating via social media to counter rumors. This requires extra work, time, and effort by the retailers for recall problems that they did not create.

There is no uniform practice for handling recalls. The Center for Science in the Public Interest surveyed 32 of America's top grocery chains to identify how retailers notify consumers about a product recall. The organization found that, while most retailers do post recall notices in their stores, the placement of these notices varies, and some chains do not post notices at all. The location and content of posted notices varied widely.

There are many database systems (e.g., IBM Food Trust, FoodloqiQ, RSM Clearthru) and blockchain technologies (Hyperledger Sawthooth, Hyperledger Fabric, Quorum, Ethereum, etc..) that are utilized to track products from the farm or manufacturer to retail locations. However, these systems fall short in collecting data about end users and notifying end users in the event of product recalls.

Every year thousands of products recalled; however, very few, if any, consumers who purchased a recalled product are ever notified directly.

Thanks to the problems with food and product supply chains, consumers are demanding more and more information about the quality and genesis of the products they consume and use. Consumers want to know where the products came from, if the product contains genetically modified ingredients, if the product is organic, if antibiotics were used in the product, whether source animals were healthy, and any safety concerns about the product.

Historically, traditional brick-and mortar relationships were incredibly personal. Consumers knew who their butcher was and over time they developed a relationship. The butcher began to understand the consumer's preferences and knew what he or she wanted before they entered the butcher shop. The butcher would also give deals based on consumer loyalty, such as a little extra on a birthday or a free sample of some new inventory. Previous generations grew up with these intrinsic relationships between consumers and their shopping supply chain. Over time, brand consolidation has increased distribution of products and dramatically increased consumer choices at the grocery store, but lost personalization.

Shoppers can be incredibly loyal to a brand (i.e., a particular company that manufactures a type of product under a particular name) and will buy that brand's products whenever they shop or dine. A shopper might buy a brand's drink at the grocery store, at convenience store while filling up their car with gas, at a restaurant when they dine out, and maybe even online. However, the brand cannot identify this shopper and is unable to reward them for their brand loyalty.

Brands have many ways by which they incentivize retailers to sell their products. A predominate method is to pay an incentive once the retailer proves they actually sold the product, also known as pay on performance. Today, retailers create manual reports detailing their performance and send these reports to brands or companies hired by the brands to manage the validation of the sales data sent via the retailer. Once sales data is approved, it can take 20-45 days to pay the promised incentive to the retailer. Brands believe that they are overpaying retailers millions of dollars in incentive payments each year. Much of this overpayment is tied to uncertain and questionable product sales reporting.

Animal and aquaculture farms are an important contributor to the human food chain. Worldwide, humankind keeps an enormous number of animals (e.g. chickens, pigs, cows, fish, lobsters, oysters, shrimps, etc.) for their egg, milk, and meat. However, there have been growing concerns about the quality of life for these animals and increasingly vocal demands for animal health, improved standards of living, and animal welfare.

Packaged foodstuffs are also an important contributor to the human and pet food supply chain. Customers want to know that the prepackaged products they are purchasing are safe and nutritious.

People each have individual habits, needs, and medical conditions. One person might have allergies to certain foods (e.g. peanuts, milk, etc.), and another person might prefer one form of communication over another (e.g. social media over text messaging, etc.).

Retail and grocery wholesalers, distributors, and retailers buy meat, seafood, produce, fruits, and other products in bulk, which are not packaged for consumer sales. These foods are portioned into sizes or weights appropriate for consumer purchases. For example, a retailer may buy a half Angus beef, then cut the beef into weights and sizes appropriate for consumer sales. These products are displayed in coolers or freezers. The retailer weighs and packages such items on electronic scales, which then print out a label detailing the weight, price, handling instructions, nutritional information, and other information. By handling and processing the product, the retailer may be directly entering the food safety liability stream and the FDA and others may now deem the retailer to be a producer and/or packager. Currently, the retailer has limited or no way of tracking these processed foods through their systems to the POS.

The current credit card industry operates from a 50+ year-old legacy model with multiple intermediaries charging excessive interest rates and fees. Banks charge card holders excessive interest rates on revolving bank credit. Payment companies charge retailers excessive interchange and transaction processing fees. Interest rates and fees on retailer-issued credit cards are even more excessive. These excesses harm the retail ecosystem and broader economy with:
(1) Interest rate charges of 15 to 30% on revolving bank issued credit card debt, which is excessive, particularly with sub 3% financing rates, sub 3% loss rates, and access to over 10× leverage, (2) Advertising 0% introductory period and applying balance transfer fees, (3) Interest rate charges of 18 to 30% on revolving retail issued credit card debt is even more excessive and most often held on bank balance sheets, (4) Interchange fees of 1 to 3%, foreign exchange fees of 1 to 5% and other fees charged by payment companies which are excessive for near riskless transactions. The credit card industry not only has some of the highest interest rates but is also one of the largest financial markets in the United States with relatively low loss rates. Purchasing volume with credit cards now exceeds over $3.0 trillion per annum in the U.S. and continues to grow at rates higher than the broader economy. Revolving credit card debt exceeded $1.0 trillion in December 2017, representing a 5.8% increase from the previous year with consumers averaging more than 3 credit and 2 retail cards in their wallet. Meanwhile, charge-offs and delinquency rates remain low, with less than 2.5% past due 30 days or more.

### SUMMARY

The disclosure relates generally to data systems and, more particularly, to a data management system wherein at least a portion of the data being stored and accessed is generated at a POS and/or retail store server and the data is stored, at least in part, on a blockchain database or other secure database. The POS may be a device at a retail store, a POS on the world wide web, a POS within an application on a mobile device, a mobile checkout POS, a cloud based POS, or any place (virtual or otherwise) where a sale or transaction is consummated, received, or verified and/or any POS, checkout, or technology that generates or verifies transactions.

The disclosure also generally relates to accessing data that is saved on a blockchain database and/or other secure databases, more particularly accessing the data for the purpose of later interacting with a consumer and/or other entity.

The disclosure also generally relates to unique ways of writing data to databases and more particularly writing to multiple databases simultaneously and automatically choosing which data is written to which database. The databases might include blockchain repositories, directed acyclic graphs (DAG), relational databases, ledgers, logs, or any other database that can securely store data.

The disclosure also generally relates to product safety, more particularly data representing products being stored on one or more databases, and the data being accessed in order to notify a consumer about a consumer safety issue.

The disclosure also generally relates to using POS devices as part of a data system to facilitate consumer and/or business services (e.g., consumer safety recalls, payments, gamification of services, personalized pricing, incentives, rewards, coupons, loyalty points, product tracking, product integrity, food providence, security, brand wallet, consumer identity safety, digital credit, etc.).

The disclosure also generally relates to using AI as part of a data system in order to quickly understand and react to data that is being gathered.

A technological solution is disclosed wherein data from POS transactions are recorded on an immutable blockchain database or other secure database. The data may be accessed by and/or provided to the payment processors, manufacturers, distributors, producers of products, and/or retailers represented by the data. The data can be used to inform purchasers of product recalls, mitigate the effects of a product recall, and provide information to purchasers and/or end users of products.

A technological solution is disclosed whereby products can be tracked in order to provide a product history to the consumer and to notify consumers if the product is defective.

Many other services can be enhanced and improved upon using a data system in which a portion of the data obtained from a POS is stored to a blockchain database or other secure database and the data is used to provide services to a consumer and/or business. The services might include new methods of providing individualized incentives to consumers or allowing brands to communicate directly with consumers and reward them for their loyalty or advertising consumption (e.g., watching product ads or information videos).

A technological solution is disclosed whereby brands can have a more personalized relationship with their customers and provide unique personalized incentives or advertising to a user in real time.

There is also need for a technological solution where artificial intelligence (AI) may be used to provide notifications, product offers, or other incentives that are more targeted or more meaningful to consumers. These interactions may be developed by an AI via the computation of data from past consumer purchases or the propensity of a consumer to buy certain products. An array of additional third-party consumer data can be attributed as well, thereby developing even greater or better targeting for the benefit of the consumer.

A technological solution is disclosed wherein a shopper may let a brand know of their loyalty when the shopper buys the brand's products often and from several locations. This allows the brand to reward the shopper as a thank you, introduce the shopper to other brand products, and incentivized the shopper to try new brand offerings.

A technological solution is disclosed that eliminates human intervention, which enables greater trust between the brand and retailer.

A technological solution is disclosed that improves food supply safety and stability by tracking, anticipating, predicting, organizing, and managing the global food supply more effectively. AI and machine learning may play an important role in some embodiments.

A technological solution is disclosed wherein AI may be utilized to alert consumers to a range of products or product types that have been recalled in the past or have a propensity to be recalled. AI may identify recall trends for products or companies and other attributes.

A technological solution is disclosed wherein AI may be used to quickly determine and notify consumers regarding individual allergens tied to products that the consumer has purchased, thus mitigating personal risk.

A technological solution is disclosed wherein AI may be used to identify how each retailer or consumer interacts in their own digital world. As a result, communications, alerts, notifications or other means of communicating may be best channeled to the consumer, thus enabling the best communication result and utilizing the most effective method to communicate with each individual consumer.

A technological solution is disclosed to help companies that handle and process food products track those food products.

A technological solution is disclosed to write weighted item product data such as meats, seafood, produce, fruit and other items from distributor, wholesaler or retailer electronic scale systems to various blockchains or other databases.

A technological solution is disclosed that originates consumer (retail) digital credit at the POS via direct payments (e.g., direct account-to-account, Automated Clearing House (ACH) payments, digital wallet to digital wallet, etc.) recorded on the blockchain (or other database method) outside of the card networks, eliminating middlemen, and offering consumers and merchants lower interest rates and transaction fees.

A technological solution is disclosed to finance, securitize, and/or distribute consumer (retail) digital credit across a broader network of lenders more efficiently via security token offerings where execution of transactions is facilitated via the representation as direct pass-through, time, credit, cashflow, and/or other tranches of the risk (cashflows) of consumer (retail) digital credit, recorded on the blockchain (or other database method).

The following summary and detailed description illustrate the best modes and preferred structures for carrying out the invention. It will be understood by persons of ordinary skill in the art that there are changes that could be made to the systems and methods that are specifically described herein and shown in the included drawings. However, for the sake of brevity, all changes that fall within the scope of the present invention have not been included in detail.

The present disclosure provides a description of systems and methods for improving consumer safety and the consumer shopping experience by providing brands with systems and methods to track their products, to issue recalls to the consumer when needed, and to provide methods to reward and incentivize consumers.

Each purpose and objective of the invention, whether stated or not, can use parts of any or all of the information and/or references contained in this disclosure and those disclosures incorporated by reference to achieve a novel application, method, system, and/or device.

One embodiment of the invention provides a unique application, method, system and/or device that combines two or more of the following objectives in the areas of consumer safety, marketing, business, and shopping:

Consumer Product Safety;

Promotions and Incentives;

Third-party incentives;

Artificial Intelligence;

Digital Credit;

Consumer Data;

System Framework; and

Supply Chain.
This list is for organization purposes only and is not intended to categorize or limit the objectives of the disclosure.

An embodiment of the invention provides a unique and novel combination of existing technology and ideas that bring hardware, software, and/or ideas together to create a novel application, product, system, or method by combining some or all of the following technologies and/or ideas: data systems, consumer safety, food safety, pharmaceutical safety, product recall methods and systems, blockchain, permissioned blockchain, encryption, gamification, incentives, coupons, loyalty rewards, points, digitized tokens, user input interfaces (e.g., keyboards, camera, haptics, buttons, brain communication, scanners, infrared signals, radio signals, WiFi, Bluetooth, near field communication (NFC), compasses, gyroscopes, accelerometers, GPS, touchscreens, graphical user interfaces, phones, computers, thermometers, thermal imaging, microphones, sensors, biometric sensing devices, facial recognition, object recognition, optical florescence, ultrasonic sensing, web searches, location data, data feeds, etc.), unique identifiers (e.g., GUID, UUID, product identifiers, etc.), security chips, AI, databases, ledgers, logs, algorithms, software, hardware, GPS, geo-fencing, beacons, radio waves, radio frequency identification (RFID), sharding, object relational mapping (ORM), object blockchain mapping, directed acyclic graph (DAG), NFC, LED enabled signals, QR codes, bar codes, machine readable graphics, scanning devices and/or methods, product integrity, tracking, food providence, animal health, genetically modified organism (GMO)-tracking, organic-tracking, antibiotics-in-food-chain-tracking, ingredient-tracking, parts-tracking, pharmaceuticals-tracking, cannabis-tracking, seed-tracking, genetic-tracking, and the like.

Consumer Product Safety

One embodiment uses a payment instrument that a consumer used to purchase a product (e.g., credit card, debit card, gift card, pre-paid card, mobile device, QR code, PayPal, payment service, etc.) to identify the consumer in order to alert the consumer of an issue with the purchased product.

Another embodiment notifies consumers of a product recall.

Another embodiment communicates with a payment instrument provider or its agent to notify consumers of a product recall.

Another embodiment identifies a consumer using an identifier (e.g., GUID, UUID, mobile device signal, unique identifier, or other identifier) that is written into a blockchain database or other database.

Another embodiment tracks product recalls.

Another embodiment matches recalled products with consumers that purchased those products and communicates with the consumers.

Another embodiment uses machine readable language (e.g., JSON, XML or other) to write information to databases, including a consumer- or transaction-identifier that is linked to products the consumer has purchased.

Another embodiment provides the ability to track purchases, link the purchase to a consumer in a database, and match a consumer that purchased a recalled product with information from a product recall notice.

Another embodiment queries databases and then alerts a retailer that there is a product in the retailer's inventory or a product that the retailer has sold that is involved in an active recall so the retailer can quickly remove affected products.

Another embodiment provides real-time updates to distributors or other relevant third-party handlers of recalled products that are still being sold so they can better manage the product.

Another embodiment tracks product recall notices, preforms a query across multiple databases that contain information about consumers and the products the consumers purchased, and matches the product recall notice to consumers that purchased a recalled product.

Another embodiment notifies consumers about a recalled product that the consumer purchased.

Another embodiment notifies consumers of a recalled product, how to handle the recalled product, and how to receive compensation for the recalled product. The compensation may be provided automatically through a payment instrument the consumer used to purchase the recalled product or another way.

Another embodiment writes recall or warning information on a transaction receipt. The information may comprise both generic and/or personalized content for a particular customer (e.g., allergens, preferences, the presence of GMOs when the product was advertised to be GMO-free, or if the product was found to be nonorganic when advertised to as organic).

Another embodiment notifies a payment instrument facilitator or it agent of a product recall and provides the facilitator or agent with information that identifies consumers either directly or via a transaction identifier associated with a recalled product, thereby allowing the payment instrument facilitator to notify the consumer of a product recall, the measures the consumer should take to protect himself or herself, and how to receive reimbursement for purchase of the recalled product.

Another embodiment provides consumers with the ability to automatically receive notices about recalled products that the consumers have purchased, and/or automatically receive reimbursement of the purchase price of a recalled product, and/or automatically receive other benefits (e.g., incentives, coupons, price reductions, free products, etc.) as compensation for having purchased a recalled product.

Another embodiment uses a blockchain database and/or other databases to track purchases and uses purchase information to inform consumers of a product recall.

Another embodiment uses a blockchain ledger to provide assurances that all transactions are valid between a real consumer and a verified retailer.

Promotions and Incentives

One embodiment leverages blockchain, a digitized, encrypted ledger that records contracts and transactions to eliminate middlemen, and brings substantial savings to coupons programs, rewards programs, temporary price reduction programs, brand/customer relationships, individualized incentives, direct advertising, consumer safety, recalls, and the like.

Another embodiment provides unique, individualized incentives, loyalty rewards, and advertising to individuals using an account, such as a digital wallet.

Another embodiment allows multiple incentives to be created and tested simultaneously.

Another embodiment tracks incentive programs in real time.

Another embodiment provides unique incentives to a user in real time.

Another embodiment provides a system in which unique targeted offers flow from brands to customers.

Another embodiment provides a system in which brands know who their best consumers are and can easily reward loyalty, provide personalized pricing, and target strategic advertising.

Another embodiment identifies a consumer's purchase then writes specific purchase information to a brand's database or blockchain for the purchase and provides the consumer rewards or incentives.

Another embodiment provides a system in which brands can identify consumers that switch out of their brand.

Another embodiment provides a system in which a brand can identify consumers that have a high potential to switch to, or become more loyal to, the brand.

Another embodiment provides a system that facilitates data sharing between brands and consumers.

Another embodiment provides a service that facilitates non-competing brands to work together to incentivize retailers and consumer to participate in marketing, incentive and programs.

Another embodiment provides a system that tracks campaigns in real or near real time.

Traditional coupon fulfillment takes weeks or months. Another embodiment provides a system that can clear coupons in one day.

Driving loyalty through continued purchases is a time-honored product discount strategy (e.g., if the consumer buys five pizzas, they get one for free). Unfortunately, most brands do not own the point of purchase and, therefore, must force consumers to make all purchases at once to get the discount or must activate cumbersome rebate or rewards programs based around entering numbers into a website.

Another embodiment provides a system that tracks consumers' activities throughout multiple retailers, purchases, and products.

Another embodiment provides a system that tracks consumers' purchases of a product or products at many separate locations.

Another embodiment provides a system that tracks consumers' purchases of products at multiple locations and provides a reward when certain purchase thresholds are met.

Many loyalty programs run the risk of incentivizing purchases that were going to happen regardless of a discount activity. Offering a consumer a $1 off on a product that the consumer was going to buy anyway is not a good business practice for a brand. Instead, an embodiment allows brands to incentivize cross-portfolio purchasing. For example, consumers who purchase $100 of a soda brand each month are incredibly loyal and do not need to be encouraged to buy more of that soda. Embodiments of the disclosure allow a brand to give that consumer a discount on other products from the same brand, thereby introducing new product lines to a valuable consumer.

Another embodiment provides a system in which brands can manage incremental purchases of product and provide individualized incentives based on purchases at multiple locations.

Sometimes a brand is not concerned about what the consumer bought, but instead what the consumer did not buy. Trip consolidation is incredibly important to consumers—most consumers try to get as much shopping completed as possible during a single trip to the store. Based on time of day, one embodiment might give the consumer a coupon to pick up pizza on the ride home because they just spent the day shopping. Another embodiment might know that it has been six months since the consumer's last oil change. In this case, a coupon can be provided in the consumer's inbox to stop at a fast lube and oil change center on the way home. Embodiments allow the brand to exist around the consumer's life but not disrupt it. This is managed through series of data permissions, so that the consumer knows exactly who has their data and for what purpose.

Another embodiment provides consumer incentives that are to be used at a second location. The incentive is selected based on a first location of the consumer and the time of day.

Another embodiment provides incentives to a consumer that are to be used at a second location. The incentive is based on the length of time that passed since a previous purchase and a first location of a consumer.

Another embodiment writes, sends, clears, and settles retailer incentives in a machine-to-machine system that eliminates human interaction, human error, and fraud.

Another embodiment writes scan-based incentive smart contracts, wherein the smart contract between a brand and a retailer is settled machine-to-machine based on sales information written to the blockchain from the retailer POS or back office systems. The smart contract is written and, when certain criteria are reached, the retailer automatically receives payment. Actual sales information is written to the blockchain from the retailer POS or back office system, and the incentive is settled when certain sales criteria are reached (e.g., sales in a certain time frame, sales goals, etc.).

Another embodiment writes retailer product sales data from a POS machine or machines at retailer's premises to a ledger on a blockchain, thereby eliminating human intervention that enables greater trust between the brand and retailer. Further, blockchain-based solutions mitigate or eliminate brand-retailer transaction fraud and accelerate payments between the brand and retailer.

Another embodiment provides a system in which retailers and brands can track and exchange scan-based offers on a trusted blockchain network while tracking sales in near or real time.

If one product is taking shelf space from better selling products and the brand will not take the slower selling product back, the retailer can control how that product is incentivized on a daily basis. Additionally, retailers can identify which products drive the greatest basket size and encourage users to purchase those products.

Another embodiment provides a system in which retailers are able to dynamically discount products based on inventory.

Another embodiment combines a permissioned blockchain with a decentralized blockchain for processing and clearing payments.

Another embodiment combines a permissioned blockchain with a decentralized blockchain for creating, processing, clearing, and paying for retailer and consumer incentives.

Another embodiment provides a system in which consumers, brands, retailers, and other partners participate in a permissioned blockchain to provide unique incentives (e.g., airline miles, fuel, movie tickets, etc.) to partner with brands for more creative and powerful incentives.

Another embodiment provides a system in which a brand gives a shopper money, such as an in-store discount, each month to be spent at a variety of stores and the shopper's purchases are tracked.

Another embodiment provides a system in which consumer packaged goods (CPG) brands can offer direct consumer incentives (e.g., rebates) for alcohol and other articles restricted by laws that may differ by area and other factors. The system knows all the relevant information and only incentivizes individuals in a manner that is legally acceptable (e.g., based on the area, consumer age, and other factors). The system can instantly incentivize (e.g., rebate) a consumer and then clear with the brand and the retailers in a matter of seconds, thereby transforming the personalized pricing of alcohol and other restricted goods, such as tobacco, inhalants, and the like.

Artificial Intelligence

One embodiment uses AI to provide a credit via a machine learning system that determines an associated incentive for each particular consumer based on a number of factors, which may include the purchase of a recalled product.

Another embodiment automatically calculates a purchase price and taxes, credits a customer's account, and provides a message back to the customer on a transaction receipt. The AI may be used to provide a credit and determine an associated incentive for each particular consumer based on any number of factors.

Another embodiment provides incentives when a consumer is near a retail location (or online) based on the consumer's past purchasing behavior. Existing hardware (e.g., computer, mobile phone, or device, GPS, beacons, NFC, RFID, LED lighting signals, or other signal technology) allows users to receive in real time, instant advertising or incentives to help them decide to buy one brand versus another brand and to receive incentives for companion products. For example, when a user buys razors, she gets an incentive to buy shaving cream, or when a user buys pasta, he gets an incentive to buy pasta sauce. This includes the ability for AI to use proximity technology combined with store product layout maps to present offers to the customer as they shop.

Another embodiment provides user incentives within the store based on proximity and signal technology.

Another embodiment uses AI, accounts (e.g., digital wallets), and a data ecosystem to provide a system in which brands can develop personalized relationships with consumers.

Another embodiment provides a system in which consumers and/or retailers share data that is read by AI to provide personalized pricing and/or incentives based on historical shopping habits.

Another embodiment predicts behavior based on other factors, such as demographics, psychographics, geo-location, and shopping patterns and delivers unique incentives based on those factors.

Another embodiment provides a system having AI-driven personal pricing, personal incentives, and personal discounts, etc. For example, a consumer may walk into her local grocery chain and the manager can say “Hello, Ms. Wilson. Great to have you back. How about a free turkey on us today? We can have it waiting at the checkout for you?” This interaction can be managed within a unique permissioned blockchain, and the consumer never has to touch a paper coupon or lift a finger.

Another embodiment provides a system in which a retailer can identify a high value shopper the moment they walk into the door of the retailer.

Millennials are now parents and are incredibly price sensitive. Trying to start a family is difficult and now more than ever people are looking for a little financial assistance. A simple coupon is only part of the equation. Every generation has used a considerable number of coupons when starting a family but being raised on personalization makes millennials a little different. It is not just the discount that matters but how that discount is delivered. Millennials have crafted every aspect of their lives and many brands have encouraged this customizability in the products and experiences they craft for today's consumer. Unfortunately, because of not owning the purchase experience, many CPG brands are unable to provide this robust personalization. Embodiments offer brands the opportunity to develop this personal experience with their consumer.

Additionally, millennials, like most generations, enjoy being rewarded for their actions. Through gamification, brands now offer these discount experiences based on milestones and rewards. Consumers are surprised and delighted by brands that they previously thought to be boring and old.

Another embodiment provides a permission blockchain system and artificial intelligence gamification of incentives.

Blockchain technology is revolutionary and provides solutions to so many encumbered systems. Another embodiment delivers solutions that many brands have been yearning for quite some time.

Another embodiment uses AI to help track and monitor the food supply chain, which allows the AI to predict issues and recommend responses in real time.

Another embodiment uses AI to help monitor product demand ensuring that retailer have enough for their customers with minimal waste.

Another embodiment improves hygiene at factories and restaurants. Cameras are used to monitor conditions in factories and restaurant and uses facial-recognition and object-recognition software to determine if workers are wearing hats and masks and complying with company procedures and food safety laws.

Another embodiment uses sensors on equipment to make sure correct temperatures are used during storage and processing and machinery is functioning correctly. If a problem is detected, the software extracts screen images or print outs of offending machinery for review.

Another embodiment helps processors with cleanliness and cleaning by using ultrasonic sensing and optical fluorescence imaging to measure food residue, as well as microbial debris on equipment in order to optimize the cleaning process and ensure that food stuff are only process with clean machinery.

Another embodiment uses computer vision and machine learning algorithms to deliver the contextual information to consumers, retailers, and others in the food supply chain.

Another embodiment uses AI to predict real-time availability of grocery items, to predict distribution of times, to predict food security outcomes, to identify food stuff, to estimate food demand, to identify fraudulent foodstuff products, to detect foodborne illness in real time, and to verify the health of individual animals (e.g., chickens, cows, pigs, aquatic animals, etc.) in the food chain.

Another embodiment uses AI for plant and seedling classification and identification of leaf diseases using images.

With new food safety regulations and the increasing need for transparency, supply chain management is a top priority for all food companies. Embodiments allow for food safety monitoring and product testing at every step of the supply chain. Another embodiment provides transparency using AI to help manage the supply chain and to assist in tracking products from farm to consumer.

Another embodiment uses sensing technologies, AI, and machine learning to automatically assess the health of individual animals and plants.

Another embodiment uses AI to predict potential issues with the supply chain by using data from news feeds, web searches, weather information, disease outbreak information, and the like

Another embodiment uses AI to draw on multiple parts of a consumer's journey to appropriately optimize experiences, conversions, and personalized promotions.

Digital Credit

In one embodiment, consumer (retail) digital credit originates at the POS, payment terminal, or back office solution via direct payments (e.g., direct account-to-account, ACH payments, digital wallet to digital wallet, etc.) recorded on the blockchain (or other database method) outside of the credit and debit card networks, thereby eliminating middlemen and offering consumers and merchants lower interest rates and transaction fees.

Another embodiment may enable a consumer to select a payment tender, which if paid on digital credit enables the consumer to maintain a debt obligation in that payment tender (essentially a short position). Meanwhile, the retailer (merchant) can select a receipt tender, which may be the same or different form the consumer's payment tender.

Another embodiment offers a solution to finance, securitize, and/or distribute consumer (retail) digital credit via security token offerings where economics of transactions are represented as direct pass-throughs, time, credit, cashflow, and/or other tranches of consumer (retail) digital credit recorded on the blockchain (or other database method) and where consumer credit may be securitized via various pooling methods by individual retailers, brands, banks, and/or credit profiles.

Another embodiment may originate consumer credit (digital credit) directly at the POS. Upon achieving critical mass, digital loan portfolios maybe sold to bankruptcy remote trusts (off balance sheet) where the digital loans will be held, serviced, and financed via the issuance of debt, equity, interest only, servicing, and other tranches. These tranches will be represented in traditional format as well as security token offerings where purchase, sale, payments, servicing, and other related transactions are recorded on the blockchain (or other database method).

Traditional Credit Card Business:

Authorization: The cardholder presents the credit card as payment to the merchant and the merchant submits the transaction to the acquirer (acquiring bank). The acquirer verifies the credit card number, the transaction type and amount with the issuer (card-issuing bank) and reserves that amount of the cardholder's credit limit for the merchant. An authorization will generate an approval code, which the merchant stores with the transaction.

Batching: Authorized transactions are stored in “batches”, which are sent to the acquirer. Batches are typically submitted once per day at the end of the business day. If a transaction is not submitted in the batch, the authorization will stay valid for a period determined by the issuer, after which the held amount will be returned to the cardholder's available credit (see authorization hold). Some transactions may be submitted in the batch without prior authorizations; these are either transactions falling under the merchant's floor limit or ones where the authorization was unsuccessful, but the merchant still attempts to force the transaction through. (Such may be the case when the cardholder is not present but owes the merchant additional money, such as extending a hotel stay or car rental.)

Clearing and Settlement: The acquirer sends the batch transactions through the credit card association, which debits the issuers for payment and credits the acquirer. Essentially, the issuer pays the acquirer for the transaction.

Funding: Once the acquirer has been paid, the acquirer pays the merchant. The merchant receives the amount totaling the funds in the batch minus either the “discount rate”, “mid-qualified rate”, or “non-qualified rate” which are tiers of fees the merchant pays the acquirer for processing the transactions.

Chargebacks: A chargeback is an event in which money in a merchant account is held due to a dispute relating to the transaction. Chargebacks are typically initiated by the cardholder. In the event of a chargeback, the issuer returns the transaction to the acquirer for resolution. The acquirer then forwards the chargeback to the merchant, who must either accept the chargeback or contest it.

Another embodiment eliminates many of the steps and processes of traditional credit card industry.

Another embodiment creates a novel digital credit system including the steps:
Establishing a credit limit: A user signs up for credit services. His credit information is searched and stored on a token and/or on a blockchain. Through a mathematical formula a credit limit is established and stored on the token and/or in the blockchain, ledger, database, and/or Directed Acyclic Graph (DAG).
Authorization: The user presents the credit payment request to the merchant via card or electronic device. A merchant device pings the blockchain, ledger, database, and/or DAG. Payment is approved or declined depending on a credit limit and used credit. Credit request and credit approval is assigned a unique identifier and stored on blockchain, ledger, database, and/or DAG.
Merchant onboarding: A merchant applies, is approved, and assigned a unique identifier that is recorded on blockchain, ledger, database, token, and/or DAG. The merchant can then accept payments.
Clearing and Settlement: On regular intervals, merchants receive funds in the currency of choice for approved credit transactions. Funds come from a trust that has raised money via security tokens or other means and is disburse automatically provided a preapproved set of criteria has been met.
Charge backs: Approved charge backs are credited to consumers digital wallet and debited to the merchant's digital wallet. If charge backs are disputed, they go to mediation. Multiple arbitrators vote over the blockchain to decide the resolution of the dispute.

Another embodiment creates a novel digital credit system including the steps:
Authorization: The user presents the credit payment request to the merchant via card or electronic device, and the merchant submits the transaction to the acquirer. The acquirer verifies the user (e.g., through a unique token, blockchain and/or other digital means), the transaction type, and the amount and reserves that amount of the user's credit limit for the merchant. An authorization will generate an approval code, which is stored on the blockchain and/or blockchain token. Some transactions may be submitted by a merchant without the user present but with prior user authorization (e.g., when the user is not present but owes the merchant additional money, such as extending a hotel stay or car rental).
Clearing and Settlement: At regular intervals (e.g., every 30 minutes), the acquirer sends batch transactions to a trust for funding. Acquirer receives funds then sends payment to merchant. The merchant receives the amount totaling the funds in the batch minus either the “discount rate,” “mid-qualified rate,” or “non-qualified rate,” which are tiers of fees the merchant pays the acquirer for processing the transactions.
Chargebacks: A chargeback is an event in which money in a merchant account is held due to a dispute relating to the transaction. Chargebacks are typically initiated by the consumer. In the event of a chargeback, the acquirer then forwards the chargeback to the merchant, who must either accept the chargeback or contest it. All is recorded on blockchain and/or database or similar.

Another embodiment replaces the credit card industry with a digital credit system that uses POS systems and blockchain for originating, tracking, and financing credit transactions.

Another embodiment provides a payment system in which multiple types of devices are all interconnected to facilitate payments with multiple types of tender including credit.

Another embodiment provides a consumer credit system that eliminates many of the processes used in the traditional credit and debit card market.

Another embodiment provides a payment system that is part decentralized and part permission based, known as a hybrid system

Another embodiment provides a consumer credit system that has only three to four entities, namely consumer, merchant, payment company, and lender (bank, finance company or trust).

Another embodiment provides a consumer credit system that has only three to four entities, namely consumer, merchant, the system, and system affiliate (lender, bank, finance company or trust).

Another embodiment clears and funds consumer credit using a permissioned based blockchain.

Another embodiment integrates into consumer credit eco-system via direct payments (e.g. direct account-to-account, ACH payments, digital wallet to digital wallet, etc.) via web and mobile devices and systems.

Another embodiment facilitates digital credit payments direct payments (e.g., direct account to account, ACH payments, digital wallet to digital wallet, etc.) between consumer's and merchant's bank accounts or between a customer digital wallet and the system.

Another embodiment offers consumers lower interest rates on new digital credit debt as compared to traditional credit card debt.

Another embodiment offers merchants lower fees on transactions paid via new digital credit debt as compared to traditional credit card debt.

Another embodiment lowers the cost of consumer credit.

Another embodiment originates consumer credit digitally at POS systems via a digital wallet application which is web and/or mobile device enabled (mobile app).

Another embodiment records digital credit transaction history for each individual or retailer on the blockchain (or other database method).

Another embodiment provides a digital wallet from which users can store, spend, and/or redeem fiat, security tokens, crypto currency, reward points, coupons, etc.

Another embodiment provides a hardware wallet from which users can store, spend, and/or redeem fiat, security tokens, crypto currency, reward points, coupons, etc.

Another embodiment provides digital wallet from which users can select the means to facilitate the tender, via debt to assets held in a digital account or via digital credit.

Another embodiment provides a digital wallet to enable customers to select the “currency” (fiat, security tokens, cryptocurrency, etc.) to maintain their digital credit debt obligation.

Another embodiment facilitates the consumers debt obligation (short position) via the credit lenders purchase of that “currency” on a listed or open exchange, rather than the tradition stock borrower and loan market, where the consumer pays interest on the debt obligation and for which the debt obligation must be repaid in the selected currency.

Another embodiment enables the retailer (merchant) to select the “currency” to receive payment in which may be different from the “currency” for which the consumer maintains the debt obligation.

Another embodiment provides multi-tender payment transaction for consumers by scanning a QR Code or bar code or similar optically unique identifier. The multi-tender transaction may include any or all the following: coupons, points, rewards, incentives, fiat currencies, crypto-currencies, credit, or a combination of any of the fore mentioned tenders.

Another embodiment provides for a consumer to pay in any combination of tenders (e.g. Fiat, security tokens, crypto-currency, points, rewards, etc..) and means (digital account or digital credit), merchant then receives payment in merchants tender of choice. Money is debited from consumers digital wallet or paid via digital credit by the consumers lender and credited to merchant's digital wallet or bank account. Merchant can keep tender in merchant's digital wallet or request through the wallet that tender be sent to merchant's bank.

Another embodiment provides for a user to store or connect to multiple tenders or representatives of multiple tenders on an electronic device, those tenders including fiat currencies, security tokens, crypto-currencies, different forms of rewards, coupons, points, incentives, precious metals, etc.

Another embodiment provides for a user to store unique keys to unlock multiple tenders or representatives of multiple tenders on an electronic device, those tenders including fiat currencies, security tokens, crypto-currencies, different forms of debit, rewards, coupons, points, incentives, precious metals (tokenized), etc. A device may display amounts for each tender the unique keys unlock. The device enables secure communication with other devices for the purposes of transferring rights to portions of those tenders to others.

Another embodiment provides a consumer (retail) credit system that is recorded on a data base and blockchain at near the same time.

Another embodiment records single and/or multi-tender transactions to a blockchain.

Another embodiment records single and/or multi-tender transactions to a blockchain and a data base simultaneously.

Another embodiment uses blockchain technology to record and initiate funding of credit transactions.

Another embodiment assigns a unique identifier to a debt that can be tracked over time.

Another embodiment assigns a unique identifier to a consumer credit transaction.

Another embodiment tracks and clears consumer credit transactions with a database and blockchain.

Another embodiment provides consumer immediate access to historical purchases.

Another embodiment provides consumers immediate access to their historical digital credit purchases by brand, product, retailer, date range and/or other criteria across the retail eco-system.

Another embodiment provides brands immediate access to their historical purchases by consumer, retailer, product, date range, and/or other criteria across the retail eco-system.

Another embodiment provides retailers immediate access to their historical purchases by retail location, consumer, product, date range and/or other criteria across the retail eco-system.

Another embodiment provides a system in which brands (product manufacturing companies) can offer digital brand credit to users of products and/or for general use throughout the consumer retail market.

Another embodiment provides a method for a brand to provide consumer credit that can be used at one or more retailers, in store or online.

Another embodiment provides a system in which brands can offer credit for consumer loyalty and/or purchases.

Another embodiment funds consumer credit with tokens (security or other).

Another embodiment provides a consumer credit system that does not use traditional banks. Note: banks may be used to pay off consumer credit debt.

Another embodiment funds digital credit debt via issuance of security debt tokens, and equity tokens, servicing tokens and/or other principal or interest only tokens or in traditional format.

Another embodiment lists these security tokens on exchanges for sale and trading.

Another embodiment represents and track ownership (title) and/or collateral on security tokens and/or a blockchain.

Another embodiment funds consumer credit via security or other tokens issued by and/or held on balance sheet by a traditional bank, finance company or trust.

Another embodiment creates security token exchange-traded fund (ETF) funds for consumer credit. In some embodiments, a DAG, blockchain, and encrypted ledger may be used interchangeably.

Consumer Data

Another embodiment provides a system in which consumer data is passed to consumer trusted sources in a transparent manner without fear of fraud or abuse.

Another embodiment provides a system in which brands have a way of clearly communicating with consumers to determine what consumer data will be leveraged, how it will be leveraged, and what is a fair value to pay consumers for that data.

Another embodiment provides a system that allows a user to provide personal information (data) to a company in return for benefits.

Another embodiment provides a system in which there are trusted exchanges between consumers and brands.

Public blockchain can work for purely public utilities like currency as it prioritizes transparency and immutability but falls short when it comes to scalability. When dealing with sensitive shopping behavior, immutability and scalability are incredibly important but transparency must be controlled. Permission-based blockchain bridges that gap.

Another embodiment provides a system of nodes (points of validation) within a blockchain so consumers know who is getting exactly what data and that data is immutable. Brands and retailers that are trusted entities become nodes (points of validation) to consumers.

Another embodiment provides a unique permission-based blockchain that uses a combination of technologies to provide unique customer experiences.

Another embodiment brings ethical transformation to the data space, in which consumer data is exchanged for value.

Another embodiment provides a system that protects consumer data by using a permissioned-based blockchain that only communicates with verified nodes and never transmits personally identifiable information.

Another embodiment provides a system that protects data that is in transit by sharding thus ensuring all data is encrypted between nodes.

Another embodiment provides a system that provides a way for brands and consumers to transact reward points, discounts, coupons, or other incentives for data. For the first time it will be the consumer at the helm of their data, and the consumer that will be rewarded for securely sharing it with brands.

Another embodiment provides transparent data sharing between brands and consumers, combined with permissioning every actor within the platform, which ensures that data is only released from consumers knowingly.

System Framework

Another embodiment enhances scalability and speed of a ledger or database system through sharding.

Another embodiment provides a system of sharding that is novel such that each player in the blockchain can have their own blockchain.

Another embodiment uses Object Blockchain Mapping (OBM) functionality that is built into a blockchain adaptor to perform blockchain sharding.

Another embodiment provides a system that has an OBM built into it so that all business objects will know how to write and read themselves to/from a blockchain in a specified format similar to the business objects being mapped to a relational database.

Another embodiment provides a system in which business objects are both an OBM and ORM capable.

Another embodiment provides a system in which the OBM will serve the purpose of isolating application developers from the underlying database technology in use (e.g., blockchain or relational). This enables modifications or changes to the underlying interfaces to the blockchain (including sidechains and the mainchain) as the blockchain environment continues to mature. This keeps application developers isolated from the technology and shields a developer from wasting resources when the technology changes.

Another embodiment provides a blockchain system in which many transactions are preformed off-chain as some transactions will not be required on the blockchain.

Another embodiment provides a system that requires transactions that are planned for the blockchain to also be in a relational database, thus writing transactions to both a relational database and a blockchain simultaneously.

Another embodiment provides a system in which an object requests data and the ORM connects to the sharded database to put the data together and deliver the data as a package without the object actually touching the database.

Another embodiment provides an ORM or mapping system in which the framework sits on top of a relational database (e.g., SQL server similar to My SQL). All database interactions are isolated away from the application developers (i.e., they make no database calls). Developers do not know that the system is using a database let alone a SQL server or any other database. Applications, Web APIs, services, and the like are built through the use of business objects that represent the business domain. Objects include wallet, address, promotion, receipt, promotion transaction, and order, for example. These objects implement the rules of the business and also encapsulate all CRUD (create, read, update, delete) operations to and from the database. Because the code is well encapsulated all database operations (CRUD) including database connection information is encapsulated as well. This can also be ORM and OBM simultaneously.

Another embodiment provides centralized connection information where horizontal database sharding is implemented. When any of the business objects (e.g., hundreds of business objects) are persisted, it asks the database connection manager where it should be persisted. The connection manager determines the type of object (order, for example) and the retail hierarchy (e.g., client, program, merchant, and store) where the order is to be persisted and returns what order database shard the order should use. Given all logic is isolated from the developer, modifications to the algorithm are easy and safe to implement.

Another embodiment provides a bridge between legacy POS systems and new technology.

Another embodiment uses the invention to write information to a customer receipt that may or may not pertain to the products being purchased and/or the method of payment.

Another embodiment writes product recall information to a customer receipt at the POS.

Another embodiment writes incentive and/or loyalty information to a customer receipt.

Another embodiment provides a system in which ORM and OBM are written near simultaneously on one or more blockchain repositories and on one or more relational databases.

Another embodiment provides a blockchain adapter by which data from legacy technology can be ported to the blockchain with minimal effort.

From production through to distribution, maintaining logistic efficiency and safety in food supply is necessary to ensure that the food supply remains stable through time. Another embodiment helps create and maintain a stable supply of food sources through the use of AI and machine learning, using generative models and genetic programming to explore food market conditions, using recommender and prediction models to analyze market factors, and using these models to drive decision making and to improve food supply stability.

Another embodiment tracks and monitors food ensuring that it is transported safely from the farm all the way to the store shelf.

Another embodiment unlocks the POS with Internet of Things (IoT) transformation to expand services, create new revenue opportunities, and awaken foundational change.

Another embodiment implements database sharding for brands, retailers, and partners. This allows groups of N number of brands in a blockchain shard and have spin off instances as they are needed (or just have one). The same process would be implemented for retailer and partners blockchain shards.

Another embodiment implements OBM as a plug-in to the data system framework as part of the blockchain adaptor. Since all applications must use the data system framework, business objects and all calls within the business objects fully encapsulate the data layer (currently SQL Server). An OBM module can be added to the data system framework, thereby easily converting all current data system framework business objects to be blockchain aware.

For instance, when a promotion is saved to the database, the calling application calls a Promotion Business Object Save method. This save method knows that it is going to a SQL Server to save its definition. It uses an algorithm to ensure the transaction is logged to the correct database shard.

The OBM plug-in works similarly and would seamlessly add an additional save operation to the blockchain. It would save to the database, and with the plug-in, the object would know its destination blockchain shard based on the transaction being brand, retailer or partner. In addition, it will know under which client, program, merchant, and site the transaction is taking place and know which shard it should use when saving to the blockchain.

Another embodiment provides a system that protects data by using the encryption benefits of the blockchain and applying blockchain sidechain sharding technology to ensure different participants in the ecosystem do not have access to data not pertinent to them. This also helps to distribute the transactions in the system away from a single blockchain and organize in such a way to meet the specific needs of each ecosystem participant.

Another embodiment provides a payment system in which a consumer identifies him or herself at a check point (e.g., by facial recognition, photograph, retinal scan, finger print, pin code, RFID, QR code, bar code, phone number, unique identifier, token, or anything similar, or a combination thereof). The consumer is able to view representations of items he or she is purchasing on his or her phone or other electronic device (e.g., as the consumer scans the items themselves or as items are scanned by a merchant, or automatically scanned via RFID, or the like), the consumer is then able to pay with a single tender or multiple tenders (e.g., coupons, reward points, incentives, fiat, cryptocurrencies, credit, etc.) The scan may occur before or after check point.

Another embodiment provides an intelligent blockchain sharding algorithm.

Another embodiment provides an intelligent blockchain sharding using multiple types of sharding. On-chain blockchain sharding will work side by side with the off-chain database sharding to give powerful and needed enterprise application functionality. In this large of a system, some transactions will be off-chain, some on-chain, and others both. Both on-chain and off-chain data services work side by side within the data system framework. Data components allow all existing business objects to inherit functionality automatically without code restructuring.

On-chain blockchain sharding works side by side with the off-chain database sharding to give powerful and needed enterprise application functionality.

Another embodiment provides a system in which some transactions will be off-blockchain, some on-blockchain and others both on and off blockchain. Both on-blockchain and off-blockchain data services work side by side within the system framework. Data components allow multiple objects functionality automatically without code restructuring.

Supply Chain

Another embodiment provides a single-source system that allows retailers to understand their total store (from inventory to register) and that facilitates purchasing, inventory management, consumer relations, and product returns.

Another embodiment provides a system that writes weighted item product data, such as meats, seafood, produce, fruit, and other items from a distributor, wholesaler, or retailer's electronic scale system to various blockchains or other databases.

The above summary is not intended to describe each illustrated embodiment or every implementation of the present disclosure.

### BRIEF DESCRIPTION OF THE DRAWINGS

The drawings included in the present application are incorporated into, and form part of the specification. They illustrate embodiments of the present disclosure and together with the description serve to explain the principle(s) of the disclosure. The drawings are only illustrative of certain example embodiments and do not limit the disclosure. Elements of the figures have been numerically labeled. Each numerical label is intended to refer to a particular element and may be cross referenced in the description of more than one drawing.

FIG. 1 illustrates a design and abstraction technique for a complex environment.

FIG. 2 depicts an embodiment of a basic two-tier architecture system.

FIG. 3 depicts an embodiment of a system using enterprise solution N-tier architecture.

FIG. 4 depicts a preferred flow within N-tier architecture according to one embodiment.

FIG. 5 depicts an organizational embodiment of a system using distributed enterprise architecture.

FIG. 6 depicts an organizational embodiment of a system with N-tier architecture layers and identity services.

FIG. 7 illustrates an embodiment of an authentication process for gaining access to a resource within a system.

FIG. 8 depicts an organizational embodiment of a system with a thin client solution.

FIG. 9 depicts an organizational embodiment of a system with a rich client solution.

FIG. 10 depicts organizational layers of an embodiment of the system.

FIG. 11 depicts an example of optimized batch processing within an embodiment of the system.

FIG. 12 depicts an embodiment in which business objects represent real entities with the system.

FIG. 13 depicts an instance of mapping business objects within the system.

FIG. 14 depicts a manner of accessing data using adapters within the system.

FIG. 15 depicts the difference between a single database, database shards, and blockchain shards.

FIG. 16 depicts an embodiment of the system showing on-chain/off-chain and multichain environments.

FIG. 17 depicts an embodiment of remote access for the system.

FIG. 18 depicts the conversion of legacy system hardware to an IoT device within the system.

FIG. 19 illustrates an embodiment of a point of sale device.

FIG. 20 depicts a computer system in which embodiments of the present disclosure, or portions thereof, may be implemented as computer-readable code.

FIG. 21 illustrates a high-level view of an embodiment of the system and some of the services it provides.

FIG. 22 illustrates an embodiment of an enterprise consumer safety system.

FIG. 23 depicts an embodiment of the system showing data flow in the event of a product recall.

FIG. 24 depicts an embodiment of the system showing data processing flows.

FIG. 25 depicts an embodiment of the system showing alerts to a customer for a recall of a purchased item.

FIG. 26 depicts an embodiment of a system that uses one or more third-party blockchains.

FIG. 27 depicts a possible flow for a product recall using a smart contract within the system.

FIG. 28 illustrates a flow of actions from a customer's purchase of a product to receiving a recall notice for the product.

FIG. 29 illustrates an embodiment of system 130 that uses on-chain, off-chain, and multi-chain technologies.

FIGS. 30A and 30B depict a transaction log 500 according to one embodiment.

FIG. 31 illustrates an embodiment of the system and its store services architecture.

FIG. 32 depicts an embodiment of the system, architecture, services, interfaces, and data flows that represent the support for personalized lifestyle and proximity-based offers.

FIG. 33 depicts a method for conversion of legacy POS systems to IoT devices that are capable of communicating with the cloud.

FIG. 34 depicts interactions between customers, retailers, and payment processors when a product recall occurs.

FIG. 35 depicts a method for writing to multiple databases simultaneously.

FIG. 36 illustrates an embodiment of the system where digital representations are being assigned to real world devices by the system.

FIG. 37 illustrates how a brand might interact with the system according to one embodiment.

FIG. 38 illustrates how a retailer might interact with the system according to one embodiment.

FIG. 39 illustrates customer interactions with a retailer and/or brand according to one embodiment.

FIG. 40 depicts a high-level view of a system for the tracking of consumer products and for the implementation of a recall via the use of a blockchain and/or other secure database.

FIG. 41 depicts business objects that reside in remote client locations, including legacy hardware as well as mobile or handheld devices.

FIG. 42 depicts how business objects that reside in the cloud map to blockchain technologies.

FIG. 43 illustrates a method for a consumer signing up to a brand wallet that allows a consumer to access coupons, discounts, and brand credit across multiple stores.

FIG. 44 represents a static system framework diagram for use with a StoreLine POS system from NCR Corporation.

FIG. 45 represents a static system framework diagram for use with an IBM POS system.

FIG. 46 represents a static system framework diagram for use with a RORC POS system.

FIG. 47 represents a static system framework diagram for use with a ScanMaster POS system from NCR Corporation.

FIG. 48 represents a static system framework diagram for use with a Mobile POS 101e system.

FIG. 49 represents a static system framework diagram for use with a cloud/online POS system.

FIG. 50 represents a static system framework diagram for use with an NCR POS system from NCR Corporation.

FIG. 51 depicts a process for locking a UPC to protect consumers from purchasing a product currently in recall from a POS device.

FIG. 52 depicts an embodiment of a digital wallet application that interacts with the system and is incorporated within phones, tablets, computers, websites and other electronic devices.

FIG. 53 depicts an embodiment of the asset-side of a balance sheet for a consumer credit lender that holds digital credit receivables on balance sheet and recorded on the blockchain.

FIG. 54 illustrates an embodiment of the digital credit process flow within a retail eco-system.

FIG. 55 illustrates an embodiment of a digital credit process flow within the retail eco-system.

FIG. 56 depicts an embodiment of the system showing electronic scale processing data flow.

FIG. 57 depicts a static view of an embodiment of the system showing electronic scale processing data flow.

FIG. 58 illustrates tracking product processing at local retail stores within the system.

FIG. 59 illustrates sensor and location data being received into the system.

FIG. 60 depicts the use of artificial intelligence as part of the system to help manage a safe food supply.

FIG. 61 illustrates the use of scan-based incentives within the system.

FIG. 62 is a flowchart illustrating a process for sending a product recall notice to a customer in an example embodiment.

FIG. 63 is a flowchart illustrating a process for stopping a purchase of a product that has been recalled in an example embodiment.

FIG. 64 illustrates a method of operation with a system framework in which a consumer receives coupons, discounts, and recommendations from both a brand and a retailer.

FIG. 65 illustrates a method of operation in which a consumer receives various benefits after purchasing a product from a retailer.

FIG. 66 is a flowchart illustrating a process for notifying a customer of a product recall in an example embodiment.

While the system of the present application is susceptible to various modifications and alternative forms, specific embodiments thereof have been shown by way of example in the drawings and are herein described in detail. It should be understood, however, that the description herein of specific embodiments is not intended to limit the system to the particular forms disclosed, but on the contrary, the intention is to cover all modifications, equivalents, and alternatives falling within the spirit and scope of the present application as defined by the appended claims.

### Glossary of Terms

Access token—credentials that define a user's scope within a system.

Adapter—a software design pattern that allows one system, network, entity, etc. to communicate with an incompatible system, network, entity, etc.

Agile software development—a software development process that emphasizes development in small meaningful deliveries with continual improvement and flexible responses to change.

AinStein—a set of remote business object components that provide remote caching and the AinStein business rules that determine order benefits. These business objects parse POS device orders and determine what benefits should be applied to an order and to a customer for later use. This component is the heart of the benefit logic and is complex logic that contains the intelligence to support all promotions.

Anonymize—to remove identifying particulars from data in order to protect the identity of a payor or for statistical or other purposes. past tense: anonymized

Application Programming Interface (API)—a set of routines, protocols, and tools for building software applications.

Application services—internal services to support the business or webservices to expose system functionality to internal and external applications.

Artificial Intelligence (AI)—the simulation of human intelligence processes by machines, especially computer systems. These processes include learning (e.g., the acquisition of information and rules for using the information), reasoning (e.g., using rules to reach approximate or definite conclusions) and self-correction. For the purposes of this disclosure, AI also includes machine learning.

Authenticated entity—a client or user that has the approval to access and/or edit an application.

Authorization—rules that determine who is allowed access to what functionality.

Automated Clearing House (ACH)—an electronic funds-transfer system run by NACHA (formerly the National Automated Clearing House Association) since 1974. This payment system deals with payroll, direct deposit, tax refunds, consumer bills, tax payments, and many more payment services in the United States.

Behavior-driven development (BDD)—a development technique that combines the general techniques and principles of Test-Driven Development (TDD) with ideas from domain-driven design and object-oriented analysis and design to provide software development and management teams with shared tools and a shared process to collaborate on software development.

Binary—a group of files in machine language code that represents or are part of an application or executable, or assembly.

Blockchain—an open ledger in which transactions between two parties belonging to the same network are stored in a secure, verifiable, and permanent way. One or more computing devices may comprise a blockchain network (or blockchain repository), which may be configured to process and record transactions as part of a block in the blockchain. Once a block is completed, the block is added to the blockchain and the transaction record thereby updated. In many instances, the blockchain may be a ledger of transactions in chronological order or may be presented in any other order that may be suitable for use by the blockchain network. In some configurations, transactions recorded in the blockchain may include a destination address and a currency amount, such that the blockchain records how much currency is attributable to a specific address. In some instances, the transactions are financial and others not financial, or might include additional or different information, such as a source address, timestamp, etc. In some embodiments, a blockchain may also or alternatively include nearly any type of data as a form of transaction that is or needs to be placed in a distributed database that maintains a continuously growing list of data records hardened against tampering and revision, even by its operators, and may be confirmed and validated by the blockchain network through proof of work and/or any other suitable verification techniques associated therewith. In some cases, data regarding a given transaction may further include additional data that is not directly part of the transaction appended to transaction data. In some instances, the inclusion of such data in a blockchain may constitute a transaction. In such instances, a blockchain may not be directly associated with a specific digital, virtual, fiat, or other type of currency. For the purposes of this disclosure, Directed Acyclic Graph (DAG) is also included in the definition of blockchain.

Blockchain/database processing engine—a system that enables adapter technology to securely write trusted data to any blockchain network or database.

Brand—a company that sells trademarked or other products that are associated with the brand. The brand manufactures, or has manufactured for them, the products, which the brand sells to consumers directly or through distributors or retailers. For the purpose of this disclosure, a brand may also be a manufacturer.

Business objects—digital representations of real-world entities, objects, and devices. The business objects define and capture business rules that represent a problem or business domain of an application. A business object represents its real-world equivalent and models relationships between real-world entities. Modeling with the real world as a template helps to organize increasingly complex enterprise systems. Digital twins (i.e., digital representations) and spaces are business objects.

Business object components—see Business objects

Business services—application services that provide critical business functions to support a system.

Business tier—also known as the business logic tier, is the physical deployment of business logic and is often made up of application services, business object components, and data technology layers.

Client—when referring to software, is a part of the system that resides in a third-party location; when referring to individuals or entities, is an external user of an application or a customer

Cloud—(or cloud computing) the on-demand availability of computer system resources, especially data storage and computing power, without direct active management by the user. The term “cloud” is generally used to describe datacenters that are available to many users over the Internet. Large clouds, which are predominant today, often have functions distributed over multiple locations from central servers (i.e., multiple servers). Clouds may be limited to a single organization (private cloud), may be available to many organizations (public cloud), or a combination of both. Also, the “system data center” or the “system cloud data center” or “system cloud” are referred to as “cloud”.

Collaboration—dependent relationship between classes in which one class depends on another to fulfill a responsibility.

Consumer product—a product that a consumer purchases or a consumer could purchase, or a product that is produced, processed, and/or transported with the intent of being sold to a consumer.

Component Based Architecture—a reusable approach in which entities are defined that contain a division of system responsibilities and the entities communicate via interfaces.

Consensus—a mechanism to ensure all participants of a network agree with a ledger edit.

Coupling—a measure of how much classes depend on one another to perform responsibilities.

Credit account—an account by which a customer obtains credit (e.g., credit card account, etc.).

CRUD task—ability to Create, Read, Update, and Delete from a data source.

Cryptographic hash—output of a hashing algorithm that validates the authenticity of information.

Customer device—any device on which a customer can receive a notification either verbal or written (e.g., phone, smart phone, computer, tablet, watch, communication device, etc.).

Customer Personal Identifying Information (PII)—unique data that identifies a customer and contains at least a name and contact information.

Customer unique identifier—information that anonymously identifies a customer without name or contact information (e.g., a number or Generic Unique Identifier (GUID) assigned to a customer). This could be a single number or a series of numbers that could be used by a payment processor to identify a customer that has a transaction account (e.g., transaction number, POS number, store number, or date).

Customer unique identifying data—is a unique data that identifies a customer and may or may not contain PII (e.g., a name and/or contact information of the customer).

Data repository—general term to describe a destination for data storage.

Data source—general term to describe the provider of data or where data is found. A data source may be any number of data repositories (e.g., a relational database, blockchain repository, etc.).

Data technology components—entities used for locating, accessing, saving, and retrieving data from a data source. These are usually adapters that allow one system, network, entity, etc. to communicate with an incompatible system, network, entity, etc.

Data tier—a physical deployment of a data warehouse, repository, or source.

Device GUID—a unique identifier for a device.

Digital replica—see Digital representation.

Digital heartbeat—an event sent by an application to indicate that it operating as expected.

Digital representation—(also digital twin, or digital replica) a virtual representation of a physical or virtual entity or device. A digital representation is a business object created and assigned to entities and/or devices (physical or virtual) that interact with and/or within the system. The digital representation's digital embodiment allows the system to model and manage the digital representation's counterpart's activity (e.g., authentication, authorization, data, processes, etc.). A digital representation might give added functionality to its physical counterpart. For example, a physical device might not be IoT compatible, but the physical device's digital representation could be IoT compatible. Spaces are a special form of digital representations. Spaces give relational structure to other digital representations. Spaces are virtual representation of a physical environment and the relationships within that physical environment. Spaces bring rational organization to a system where the physical environment is replicated in digital form. A retail store that interacts with the system might have a legacy POS device (i.e., not IoT compatible). The system would assign digital representations to both the retail store and the legacy POS device. The retail store would be a space (special digital representation) and the legacy POS device would be a digital replica of the legacy POS device with added features (e.g., IoT ready, etc.). If a POS is a virtual POS, the system might assign a digital representation to the virtual POS that is a digital replica of a server or other digital representation that the system can then track and manage. A digital representation can be as simple as an identifying number or much more complex.

Digital twin—see Digital representation.

Distributed Enterprise Architecture (DEA)—a design framework that leverages remote client tiers to distribute functionally, limit server calls, balance processing, and reduce the risk of downtime.

Domain-Driven Design (DDD)—an object-oriented analysis and design technique that takes the ubiquitous language of the domain and represents it in domain objects. DDD connects these domain objects to an evolving model of core business concepts.

Endpoint operation—a connection point to a webservice over a network to perform a function.

Entity—a part, device, component, module, repository, system, individual, or business.

Execution package—application or code modules that work together to solve a system objective.

Facade—a software design pattern that provides a simpler interface to more complex underlying interfaces.

Food safety—for the purposes of this disclosure, food safety is any activity associated with attempting to protect humans or animals against illness, danger, risk, injury, or death resulting from any consumer product. The product could be intended for use by animals or humans, such as a food item, drug, pharmaceutical, vitamin, supplement, etc. or a non-food item.

File data source—a data source managed by a file system.

Graphical User Interface (GUI)—a visual representation of a presentation layer.

GUID—Generic Unique Identifier—a unique identifier that may not contain personal identifying information (PII).

Horizontal partitioning—distribution of the same type of data across multiple physical or virtual environments.

Identity services—an application layer dedicated to controlling access to an application through the management of authentication and authorization.

Internet of Things (IoT)—the network of physical devices, vehicles, home appliances, and other items embedded with electronics, software, sensors, actuators, and connectivity that enables these things to connect, collect, and exchange data.

IoT Hub—a remote business tier that manages communication with IoT devices or legacy hardware and cloud services.

JSON (JavaScript Object Notation)—an open-standard file format language that uses comprehendible text made of attribute-value pairs to transmit data objects.

Layer—partitioned code within a tier to separate or decouple objects and functions.

Ledger—a distributed ledger of shared and synchronized data within blockchain.

Legacy hardware—objects without the inherent ability to externally communicate.

Libraries—physical representations, such as a software development kit (SDK) or an application programming interface for either a web server or a web browser (Web API) that makes available groups of classes with subsystem properties to other development teams for reuse.

Light architecture—associated with two-tier architectures where software layering is minimal or non-existent.

Log—list or wallet in which specific information is stored.

Machine-to-Machine (M2M)—direct interaction between machines, such as M2M communication, M2M clearing, and M2M settlement. M2M eliminates human error, fraud, and middlemen. For the purposes of this disclosure, M2M also includes machine-to-system or system-to-machine.

Manufacturer log—a log (such as a digital wallet) that is identified by a product identifier and that stores customer information of customers who purchase the product identified by the product identifier. Also called product log.

Multichain blockchain—the ability to write to multiple independent blockchains and manage several consensus models without having to rewrite data or code.

Namespace—name to uniquely identify a group of like objects.

Node—device on a blockchain network that stores a copy of the data.

N-Tier—a system structure having distinct layers that interact through collaborations or interfaces. Each layer can be independently updated and shared. N-Tier most commonly references class grouping around (1) presentation, (2) business layer, and (3) data.

OAuth 2.0 (RFC 6749)—an open access standard and authorization framework process to provide users access to an application.

Object-Blockchain Mapping (OBM)—an adapter that maps business object properties to a blockchain ledger.

Object-Relational Mapping (ORM)—an adapter that maps business object properties to a relational database.

Object-Webservice Mapping (OWM)—an adapter that maps business object properties and actions to RESTful webservice endpoints.

OO—acronyms for Object-Oriented.

OOA/D—acronyms for Object-Oriented Analysis and Design.

Oracle—trusted third-party that finds and verifies real-world information and then transmits that information to the blockchain.

Order harvester—a system that automates complex data normalization so multiple sources are converted to a standard format (e.g., standard system language format).

Payment instrument—an instrument provided by a payment processor or their agent (e.g., bank, credit union, credit card company) may be in the form of a credit card, application, or any instrument that can be used to facilitate payments against an account. If an instrument is approved for use by a payment processor, it is considered to provide by the payment processor.

Payment instrument account—an account associated with a payment instrument. The account tracks purchases, returns, credit limits, used credit limits, and payments made with the payment instrument.

Payment processor—an entity that establishes (e.g., opens) a letter or line of credit in favor of a beneficiary, and honors drafts drawn by the beneficiary against the amount specified in the letter or line of credit. In many instances, the issuer may be a bank or other financial institution authorized to open lines of credit. In some instances, any entity that may extend a line of credit to a beneficiary may be considered an issuer. The line of credit opened by the issuer may be represented in the form of a payment account and may be drawn on by the beneficiary via the use of a payment card. An issuer may also offer additional types of payment accounts to consumers as will be apparent to persons having skill in the relevant art, such as debit accounts, prepaid accounts, electronic wallet accounts, savings accounts, checking accounts, etc., and may provide consumers with physical or non-physical means for accessing and/or utilizing such an account, such as debit cards, prepaid cards, automated teller machine cards, electronic wallets, checks, etc. The payment processor may be any company that acts as an intermediary between a payor and a payee (e.g., a credit card company, credit card network, payment service company, contract company that has been employed to facilitate payments, bank, any company that facilitates payments between entities, etc.). The payment processor may include a system or network used for the transfer of money via the use of cash-substitutes for thousands, millions, and even billions of transactions during a given period and may use a variety of different protocols and procedures in order to process the transfer of money for various types of transactions. Transactions that may be performed via a payment network may include product or service purchases, credit purchases, debit transactions, fund transfers, account withdrawals, etc. Payment networks may be configured to perform transactions via cash-substitutes, which may include payment cards, letters of credit, checks, transaction accounts, etc. Examples of payment processors including networks and/or systems configured to perform as payment networks include those operated by MasterCard, VISA, Discover, American Express, PayPal, WECHAT, VENMO, a crypto currency network, etc. Use of the term “payment processor” herein may refer to both a payment network as an entity and a physical payment network, such as the equipment, hardware, and software comprising a payment network.

Payment rails—infrastructure associated with a payment network used in the processing of payment transactions and the communication of transaction messages and other similar data between the payment network and other entities interconnected with the payment network that handles thousands, millions, and even billions of transactions during a given period. The payment rails may be comprised of the hardware used to establish the payment network and the interconnections between the payment network and other associated entities, such as financial institutions, gateway processors, etc. In some instances, payment rails may also be affected by software, such as via special programming of the communication hardware and devices that comprise the payment rails. For example, the payment rails may include specifically configured computing devices that are specially configured for the routing of transaction messages, which may be specially formatted data messages that are electronically transmitted via the payment rails, as discussed in more detail below.

Payment transaction—a transaction between two entities in which money or other financial benefit is exchanged from one entity to the other. The payment transaction may be a transfer of funds for the purchase of goods or services, for the repayment of debt, or for any other exchange of financial benefit as will be apparent to persons having skill in the relevant art. In some instances, payment transaction may refer to transactions funded via a payment card and/or payment account, such as credit card transactions. Such payment transactions may be processed via an issuer, payment network, and acquirer. The process for processing such a payment transaction may include at least one of authorization, batching, clearing, settlement, and funding. Authorization may include the furnishing of payment details by the consumer to a merchant, the submitting of transaction details (e.g., including the payment details) from the merchant to their acquirer, and the verification of payment details with the issuer of the consumer's payment account used to fund the transaction. Batching may refer to the storing of an authorized transaction in a batch with other authorized transactions for distribution to an acquirer. Clearing may include the sending of batched transactions from the acquirer to a payment network for processing. Settlement may include the debiting of the issuer by the payment network for transactions involving beneficiaries of the issuer. In some instances, the issuer may pay the acquirer via the payment network. In other instances, the issuer may pay the acquirer directly. Funding may include payment to the merchant from the acquirer for the payment transactions that have been cleared and settled. It will be apparent to persons having skill in the relevant art that the order and/or categorization of the steps discussed above performed as part of payment transaction processing.

Permissioned blockchain—a private blockchain with an access control layer built on the protocol to manage, authenticate, and authorize participation.

Point-of-sale (POS)—sits at the center of the system framework. It is a computing device or computing system configured to interact with a user (e.g., a consumer, employee, etc.) for receiving transaction data, payment data, and/or other suitable types of data for the purchase of and/or payment for goods and/or services. The POS may be a physical device (e.g., a cash register, kiosk, desktop computer, smart phone, tablet computer, etc.) in a physical location that a customer visits as part of the transaction, such as in a “brick-and-mortar” store, or may be virtual in e-commerce environments, such as online retailers receiving communications from customers over a network such as the Internet. In instances where the POS may be virtual, the computing device operated by the user to initiate the transaction or the computing system that receives data as a result of the transaction may be considered the POS, as applicable. Legacy POS systems (i.e., those that are not IoT compatible) are converted by the system framework to IoT devices. The legacy system devices become a business object that model physical IoT devices in digital form. These business objects may communicate with blockchain technologies using a data technology OBM adapter component. The OBM allows business objects to be loosely coupled to blockchain technologies.

POS GUID—a unique identifier for a POS.

Presentation services—user interface to an application.

Presentation tier—physical deployment of a user interface to an application.

Primary key—relational database field that will uniquely identify table records.

Product log—see Manufacturer log.

Product recall—a recall notice usually provided by a brand or government agency.

Proof of authority—blockchain consensus algorithm in which transactions are validated by approved accounts.

Proof of stake—blockchain consensus algorithm in which transactions are validated by highest stakeholders.

Proof of work—blockchain consensus algorithm in which transactions are validated by miners who compete to complete transactions and get rewarded.

Propagate data—generating data from information. Propagated data may be exactly the same as the information from which the propagated data was generated.

Public blockchain—an open blockchain network that allows for public participation and access.

Recall notice—a communication that provides information related to negative issues concerning a product. The recall notice content may be as simple as product name.

Relational database—organization of data that maps to business objects and physical spaces.

Remote—location outside of the cloud venue where the primary system resides. Otherwise known as a client location.

Remote business tier—a lightweight representation of a server business tier Remote business tiers are deployed at client locations to execute lightweight business processing rules and provide access to a server business tier.

Remote presentation tier—part of a distributed enterprise architecture that represents the deployment of a presentation tier at a client location to help support local processing and mitigate server calls.

Repository—place or combination of places to store data. Data can be stored across several places or in one single place (e.g., blockchain repository, database repository, etc.)

Representational state transfer—definition of the REST acronym that is associated with the architectural style used for web services. Used to provide interoperability of systems over the internet.

Responsibility—unit of work that relates to the functional requirement of the system. The functional requirements are made up of many responsibilities distributed intentionally across classes.

Responsibility-Driven Design (RDD)—an object-oriented analysis and design technique in which objects play specific roles. Each object is accountable for a specific portion of the work. Objects collaborate in clearly defined ways, contracting with each other to fulfill the larger goals of the application. By creating such a “community of objects,” assigning specific responsibilities to each, developers build a collaborative model of your application.

RESTful—acronym for Representational State Transfer that is associated with the architectural style used for creating web services. Used to provide interoperability of systems over the internet.

Rich client—a more robust client that manages many functions without communicating with the server business tier.

Server—primary host of an application managed by the application architect.

Service-Oriented Architecture (SOA)—architectural methodology that segments business features in services that provide a cohesive set of functionalities. These services provide the capability to others via a communications protocol typically over a network. This promotes an environment of loosely coupled services that can be shared, reused, and combined to build production applications that provide a larger set of functionalities.

Shard/Sharding—horizontal partitioning of data across multiple database servers or physical locations.

Smart contract—a computer protocol intended to digitally facilitate, verify, or enforce the negotiation or performance of a contract. Smart contracts allow the performance of credible transactions without third parties. One of the best things about smart contracts on a blockchain is that, because it is a more decentralized system that exists between all permitted parties, there is no need to pay intermediaries (i.e., middlemen) and it saves time and conflict. Executing smart contracts on a blockchain is undeniably, faster, cheaper, and more secure than traditional systems. Smart contracts help to exchange money, property, shares, vote, or anything of value in a transparent, conflict-free way while avoiding the services of a middleman.

SOAP—acronym for Simple Object Access Protocol, a messaging protocol specification for exchanging data using XML with web services.

Socket data sources—data providers whose primary means to send and receive data happens through sockets.

Sockets—an inter-process communication mechanism in which a socket is an endpoint of a two-way communication link. Sockets allow communication between processes on the same or different machines in diverse environments.

Software Development Kit (SDK)—library of shared code available for reuse to build larger systems.

Spaces—digital representation of a physical environment and relationships within that environment. A space is a business object.

Subsystem—group of classes with a high degree of dependency through common responsibilities that fulfill a greater distinct system purpose. These subsystems can often be converted into libraries.

Test-Driven Development (TDD)—development technique in which tests are written before anything else. The goal is to capture the specification with a set of small (positive and negative) unit tests. Code is then written and run on the unit tests.

Thin client—light client that relies on the server for all major business and data processing.

Till controller—an agent or API on a store back office server (can also be located in the system cloud on the IoT Hub) that communicates with the POS and with the cloud. The till controller serves as the remote IoT Hub to manage all legacy POS devices. The till controller processes real-time scans at the register to determine rewards (e.g., coupons, points, third-party rewards) to be added to the order, communicates back to the cloud on rewards settled, and adds rewards for both loyalty and non-loyalty customers. This is a benefit engine that manages all POS systems in a store and the order activity occurring at checkout. The till controller communicates with the cloud for wallet information and returns to the POS systems wallet information and benefits to be added to the order. The till controller also manages and assigns digital representations of the POS devices. The till controller's activity can be monitored in real time where POS-IoT events can be captured and provided to the datacenter where cloud services can provide a view of till controller IoT hub activity. This API can also be used by third-party loyalty systems to integrate functionality.

Tier—physical deployment of an application with many layers.

Tightly coupled—concept in which modules of a system are highly interrelated and dependent on one another.

Transaction account—a financial account that may be used to fund a transaction, such as a checking account, savings account, credit account, virtual payment account, etc. A transaction account may be associated with a consumer, which may be any suitable type of entity associated with a payment account, and may include a person, family, company, corporation, governmental entity, etc. In some instances, a transaction account may be virtual, such as those accounts operated by PayPal or the like.

Transaction Log (TLog)—a complete, detailed record of everything that occurs at the POS terminal, including events that are not directly related to a sales transaction. Typically, the TLog record format is unique to a given POS application.

Two-tier architecture—a solution for simple systems with a presentation tier and a data tier.

Unified Modeling Language (UML)—a general-purpose, developmental, modeling language in the field of software engineering, which is intended to provide a standard way to visualize the design of a system.

Unique identifying information—any information that identifies or helps identify a person, place or thing. The information might contain PII (e.g., name, address, phone number, etc.) or it may be without PII (e.g., GUID, transaction number, POS number, store number, date, and/or other unique number or combinations of numbers (including letters) given only to that person). Unique identifying information may be obtained from anywhere (e.g. machine-readable mark, RFID, facial recognition processes, product recognition processes, payment instrument data, etc.).

Unique identifying data—data that is or was propagated from unique identifying information. Unique identifying data may be machine readable.

Universal Product Code (UPC)—for the purposes of this disclosure, a UPC is any product identifier (e.g., RFID tag, QR Code, UPC code, machine-readable marking, etc.).

User exit—a subroutine that is invoked by a software package for a predefined event in the execution package. Clients of the package can substitute their own subroutines in place of the default ones provided by the package vendor to provide customized functionality.

Web API—an API similar to an SDK that is available on the Internet and more commonly used for business-to-business partnerships.

Web API connection—a service that allows clients and partner blockchain and/or database networks to access clean, trusted data through standard communication protocols.

Webservice—an application service that publishes business object components to allow internal and external application and business services to reuse and share business logic.

XML—Acronym for eXtensible Markup Language, which is a set of rules for a document format.

### DETAILED DESCRIPTION

Object-Oriented (OO) Analysis and Design (OOA/D) techniques have been around since the late 1980s and early 1990s. These techniques evolved and improved greatly during this time by the likes of Grady Booch, Peter Coad, Ivar Jacobson, Steve Mellor, Bertrand Meyer, Jim Rumbaugh, and Rebecca Wirfs-Brock. OOA/D simplifies complex problems to bring understanding and order.

When analyzing a complex problem, there are many pieces that need to collaborate in different ways. When these pieces seem to have no commonality in their interaction, they are an unorganized complex problem. To bring order, one must analyze a myriad of states and actions that make it impossible for an individual to track all the details. Software systems continue to grow in complexity but there are limits in the ability to manage and cope. A system must first be broken into smaller, digestible parts to understand it. In doing so, the human limitation to manage complexity is overcome and the problem is understood in parts and not all at once. This is known as abstraction.

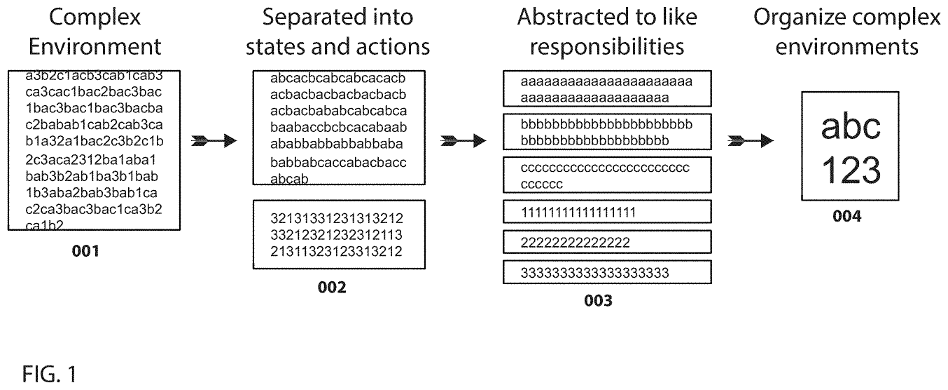

Through the process of abstraction, complex environments are simplified to allow for elegant design. Implementation of this strategic process is clear, clean, and intentional. FIG. 1 illustrates a design and abstraction technique in which a complex environment 001 is separated into states and actions 002 that are further abstracted to like responsibilities 003 and then further abstracted to fully organize complex environments 004.

By using the concept of abstraction, parts of a complex problem are investigated, understood, and captured within objects. Each object contains definition information and knows how to perform operations. In Wirfs-Brock's book, Designing Object-Oriented Software, she offers a technique known as responsibility-driven design to break complex problems into simple manageable pieces. It focuses on taking a system's responsibilities and organizing them into objects where objects work together to solve the greater system responsibilities. Object responsibilities become interfaces and business rules to be implemented. These objects become holders of logic and data to provide services and interfaces to others. Each object plays a role and must do its part in supporting the solution. The responsibility-driven approach is a very natural way to solve software problems.

In Responsibility-Driven Design (RDD), objects are defined and organized into an even higher-level abstraction groupings. These object groupings are subsystems or layers where the objects within each group are tightly coupled or highly collaborative and dependent on one another to perform their responsibilities. These layers can then be packaged and managed independently of other layers. This approach allows systems to be easily understood and easily planned and to have even work distribution, fewer programming issues, quicker implementation time, fewer maintenance issues, and less invasive new technology adoption.

One of the most recent technology changes is Service-Oriented Architectures (SOA). Systems built using SOA and OOA/D principles now have greater integration possibilities than ever before. SOA exposes objects over a network where different systems use different implementation technology and can communicate or leverage functionality for even greater interoperability. Problems once impossible to solve are now solved. The great work of the OO forefathers is the bedrock for technological advancements, such as SOA.

Enterprise architecture is the organization of complex applications and/or services. Enterprise systems demand an architecture that properly organizes necessary business services into multiple tiers and layers. Tiers represent the physical segmentation of a system that contains discrete layers within them. Each layer signifies a service or component of a tier. Services and components are the business processes that allow a system to execute. More advanced enterprise systems may also require distributed services that balance the processing load and allow for localized business services.

OOA/D techniques help understand increasingly complex systems by encouraging developers to think about real-world aspects of a problem. Business problems are analyzed and organized into smaller subsystems to help manage and model the system. These subsystems are transformed into layers of components and services for easier management of a system within tiers.

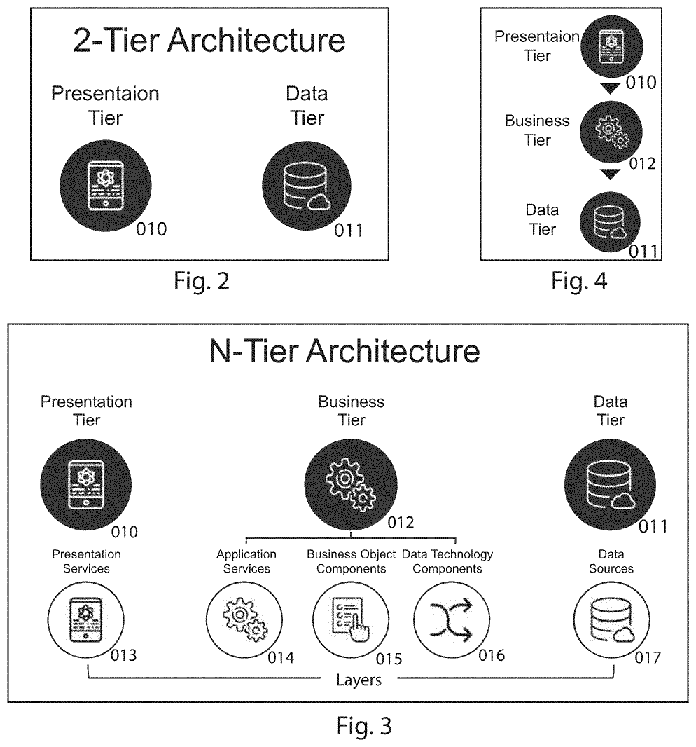

FIG. 2 depicts an embodiment of a basic two-tier architecture system. It consists of a presentation tier 010 and a data tier 011. Functional two-tier programming is a solution for simple systems, but more complex business needs require a third business logic tier that manages specific business functions.

FIG. 3 depicts an N-tier architecture, which is a departure from more traditional two-tier architectures (FIG. 2). The two-tier architecture places significant loads on networks due to heavy interaction between client and server. This may be adequate for the controlled environment of a corporation but is difficult when applications are accessed over the Internet. Two-tier architectures do not scale well and typically must be rewritten or completely replaced when they reach capacity. Without the ability to decouple business logic from the presentation and database logic, applications cannot migrate to different presentation devices, add new functionality, or integrate with other applications.

FIG. 3 depicts an embodiment of a system using enterprise solution N-tier architecture. The “N” being any number 3 or greater. To capture enterprise business functions, additional layers are developed within the added business logic tier. This is more commonly known as a three-tier architecture. The three tiers being the presentation tier 010, the business tier 012, and the data tier 011. Business components 014, 015 and 016, services 013, and data technology 017 are organized into separate layers. This segments business logic from data for easier management.

N-tier architecture, more specifically the traditional three-tier architecture, partitions systems into presentation logic 010, business logic 012, and data management 011. This adds a business logic tier 012 to the two-tier system and decouples business logic from the presentation tier 010 and data tier 011. This additionally allows business logic 012 to be shared amongst applications through webservice and component interfaces. When properly organized, the software and hardware for each tier is managed independently of each other.

FIG. 4 depicts a preferred flow within N-tier architecture according to one embodiment. The presentation tier 010 is the topmost level of an application interface for users or programs. Its primary function is to translate tasks into something users understand. This tier can have multiple components and includes responsibilities such as GUI presentations, mobile applications, or IoT connections.

The business logic tier, or business tier 012, performs the business operations for a programmed system. This includes communication coordination, command process, function execution, logical decision making, and processes data between the presentation and data layer.

The data tier 011 stores, manages, and provides access to data. This is done with a single database, file system, blockchain, or multiple variations or combinations for more complex systems.

Functional programming has streamlined many technology needs to simple two-tiered architectures. Simple systems are managed through this light architecture without being troubled by establishing sound architecture. However, large enterprise solutions demand structure to appropriately scale. This is accomplished by developing tiers that organize business needs within the architecture, most often an N-tier or multi-tier architecture. N-tier architecture is a client-server architecture in which the tiers (presentation 010, business logic 012, and data 011) are physically separated. A tiered approach allows different layers of software to be moved amongst tiers to maximize business benefit.

Some of the benefits of N-tier architecture are scalability, easily maintained code, shareable components, load balancing, upgraded tiers and layers independently, rapid technology adoption, business tier reuse, hardware and software flexibility, security, and more.

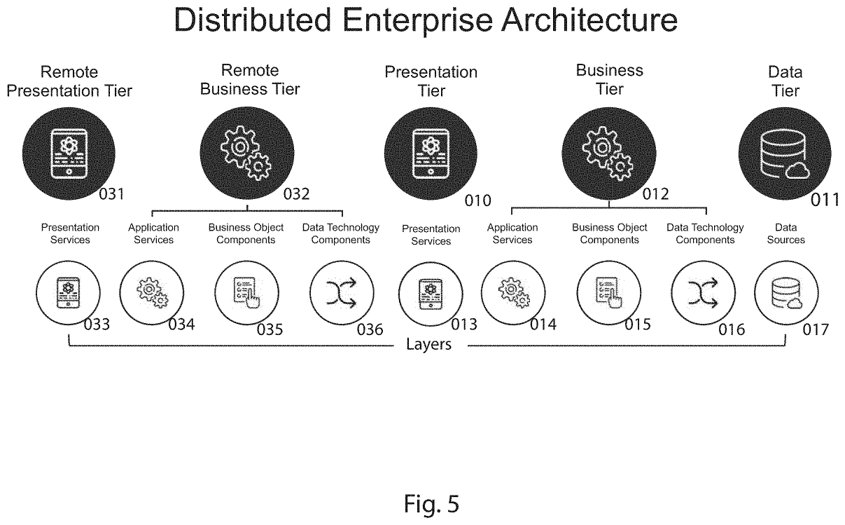

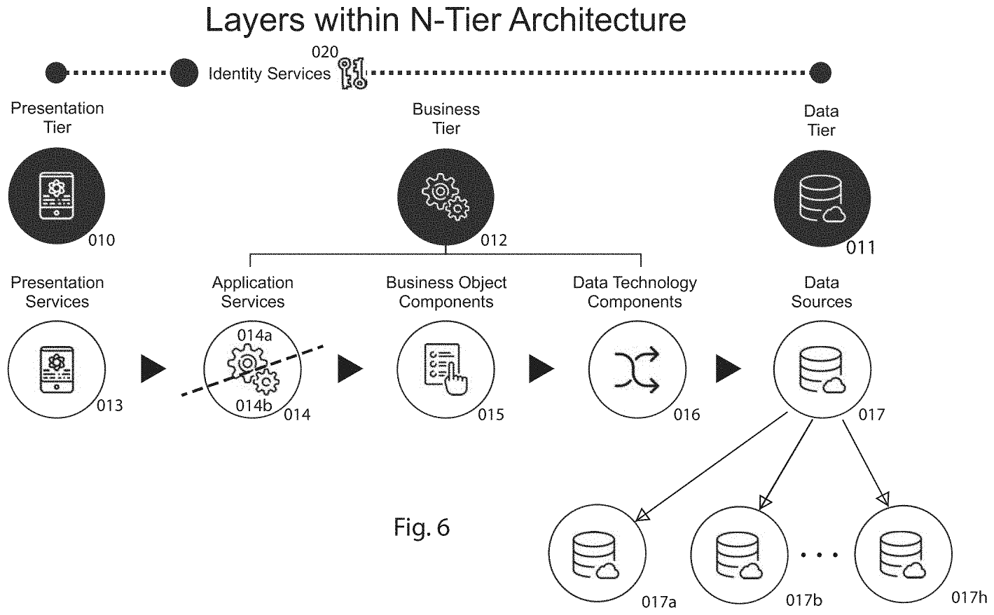

FIG. 5 depicts an organizational embodiment of the system 130 using Distributed Enterprise Architecture (DEA). DEA systems require additional remote client services to manage local processing. This adds additional remote presentation 031 and remote business 032 tiers to the existing server tiers to the N-tier architecture. The remote business tier 032 and the remote presentation tier 031 can interface with their respective layers that include remote presentation services 033, application services 034, business object components 035, and data technology components 036.

FIG. 6 depicts an organizational embodiment of system 130 with N-tier architecture layers and identity services. It shows the arrangement of services and components within tiers. Tiers imply process and/or network boundaries. Layers are the organization of code in services or components. Tiers are the physical deployment of these layers. Layers include presentation services 013, application services 014, business object components 105, data technology components 016, and data sources 017.

Organizing code in layers offers the benefit of code reuse, system stability, easier maintenance, easier team management, shorter development cycles, and lower development costs. Organizing the layers within tiers provides the benefit of performance, scalability, security, and fault tolerance. Layers are placed in the appropriate tier to maximize the needed benefit.

Identity Services 020 control access. The identity service models a system's complex interactions between people, places, and things to support authenticated interactions and robust spatial intelligence.

Presentation services 013 provide an interface to other services. Proper exposure and extension to the application services within a user-friendly workflow is done through a presentation such as an IoT device or GUI-like mobile or web applications.

Application services 014 share and execute business functions. Application services are both webservices 014a and business services 014b. Webservices 014a are public and used by customers to integrate with business object components through RESTful APIs. Private services that are not externally exposed but are responsible for critical business support functions are business services 014b.

Business object components 015 define and capture business rules. Objects that represent the problem or business domain and contain data or rules are business objects. Business object components 015 are the backbone of component architecture.

Data technology components 016 find and access data. Systems use various forms of data sources or repositories to save or retrieve data. Data technology components 016 are responsible for locating and accessing the data in the required protocol of the data source. This includes access to file shares, webservices, blockchain, relational, and other databases.

Data sources 017 are the collection of data repositories and files. Large enterprise data-intensive systems organize and define required data sources to store data within relational databases, blockchain, or file shares. Data sources 017 define their required structure from data requirements in the business object components.

Applications and services 014 within a system manage complex interactions between people, places, and things. These interactions must be authenticated, authorized, and requires robust spatial intelligence to give context to operations. The identity service 020 provides these services to the system.

A centralized security management service within identity services 020 contains information on users and their roles, API key authorization information, data source details, application and business service information, and physical space definitions for devices that users utilize to access system services.

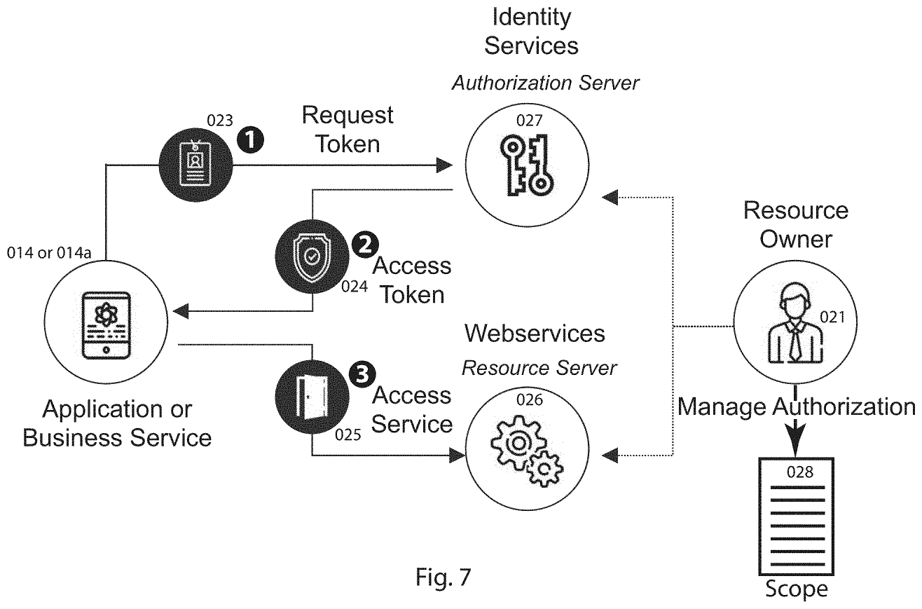

Application or business services 014 are authorized to use system resources found on resource server 026 (FIG. 7) once they are successfully authenticated. This is done through traditional user identifier and password and/or API access token.

Presentation services 013 are both graphical and non-graphical presentations to system users and are responsible for giving the best experience to the user. To properly present business functions to users, presentation services 013 interface with webservices 014a to access business component data and rules. Presentation services 013 commonly come in the form GUI applications, such as desktop, web, and mobile, or on non-GUI objects, such as IoT devices, loyalty cards, or RFID chips.

In SOA, webservices 014a are the means to publish business object components 015 over a network. Webservices 014a allow internal and external applications and business services 014b to reuse and share business logic.

Webservices 014a provide similar advantages as business object components 015, such as interoperability and loose coupling to enable high reuse and development efficiency. A powerful difference is webservices 014a are provided over a network and are technology independent. This allows webservices 014a to be shared across businesses and provides a new level of interoperability.

In this scenario, a bridge is created to the business objects. Presentation layer application 013 or business service 014b makes a request to webservice 014a. Webservices 014a delegate the request to one-to-many business object components 015. The webservice 014a determines the JSON or XML response granularity. Business objects components 015 perform the operations. A response is returned in standard JSON or XML structure.

When communicating with business object components 015, a webservice 014a publishes business object components 015 through an interface using RESTful technology. They use the standard HTTP operations, such as GET, POST, PUT, and DELETE, to expose appropriate business object components 015 through endpoint operations. The business objects are referenced using REST resource naming that maps to a business object component name. This enables developers to more easily understand and adapt to business terminology and services.

A webservice 014a uses the Facade pattern for each business object component 015 published. The Facade is responsible for strategically publishing only those business object component 015 operations needed by the webservice 014a. Webservice 014a endpoints map to business object 015 operations are either coarse-grained or fine-grained. Coarse-grained endpoints return more data than fine-grained endpoints but have the advantage of reducing roundtrips to the service for data. By default, endpoints are coarse-grained but can be converted to fine-grained or additional endpoints can be added to make endpoints as fine-grained as necessary.

The Façade pattern implements webservice 014a endpoints and delegates endpoint work to the appropriate business object component 015. Business object components 015 perform the operation and, when appropriate, return their response in either JSON or XML format. Data is transferred to and from the consumer in these formats. Business object components 015 allow a component consumer to control JSON and XML granularity. This includes what properties and aggregated children should be serialized.

Regarding business object component 015 format, different webservices 014a that delegate to the same business object component 015 use the same JSON and XML general structure as defined by the business object component 015. The granularity of data returned by these differing webservices 014a can be customized by the business object component 015 interface. In enterprise systems, a standard JSON and XML structure for each business object component brings a higher degree of interoperability and reuse across all webservices 014a and removes the additional work of mapping different formats.

Responsibilities or tasks of a system that do not logically sit within the business object components are placed within business services 014b. This layer distinguishes itself from other services by not supporting mainline front-end business actions. Business services 014b are responsible for business support services. These services are independent executables and include self-hosted Web API services.

Business services 014b are defined by tasks where one-to-many business objects 015, data technology adapter components 016, and application webservices 014a work together to perform a valuable business function. Business services do not remove or duplicate business logic from business object components but utilize this business logic in tasks that relate business objects 015 to other internal or third-party webservices and processes. Business services add building block functionality to the system and are therefore considered part of the business tier 012.

Typical business services would include functionality to receive or send data to third-party systems, strategic system caching, an enterprise service bus (ESB), high volume batch processes such as data import or export, system notifications, and data management processes.

Business objects components layer define objects in the business domain. Classes that represent the problem or business domain and collaborate to perform the business are business object components 015. Some authorities, such as Eric Evans in Domain Driven Design, refer to these classes as domain objects. In either case, these classes represent the business logic layer and encapsulate the business intelligence of a system. Well-designed business objects with proper encapsulated functionality are shared across many system applications and services and are the backbone of a component architecture that powerfully support SOA.

ORM maps business objects 015 to relational databases.

Business objects 015 contain data, business logic, and relationships that represent the business domain. Business objects 015 interface with the data technology components to fully encapsulate data source operations.

The primary source of data used by business objects 015 are relational databases. There are several libraries and tools available to allow business objects 015 to interface with relational databases. One of the more popular tools is ORM. It assists in mapping objects to relational database structure and removes the burden of coding repetitive database CRUD tasks for business objects 015. The business objects 015 use an ORM available as a data technology component 016. This ORM encapsulates data access and keep it loosely coupled to business objects 015.

ORM database shards.

The ORM, in collaboration with the identity service 020, provides all required database intelligence along with an interface to support common business data functions. Namespaces help organize business objects 015 where highly cohesive business objects 015 reside in a common namespace. The ORM uses this namespace with the identity service 020 and its space definition to determine the database shard to execute data source operations. This is a powerful method to map business objects to data sources.

There are sources of data for business objects 015 other than relational databases. This requires the use of other data technology components 016 to communicate with these data sources. They include sockets, files, third-party webservices, and emergent blockchain technology. Business objects can integrate with these technologies through the data technology adapter components.

Systems use various forms of data sources 017 to save or retrieve required data. The data technology layer 016 is responsible for providing components that perform this work. These components use specific technology APIs for relational databases, files, webservices, blockchain, or socket-based data sources.

Business objects 015 and business services 014b are loosely coupled to data sources 017 by separating the data technology complexities from the business logic. This allows businesses to easily manage and integrate new data sources 017.

Sharding is a database management technique to help distribute the load of a system by breaking larger database systems 017 into smaller databases (e.g., 017a, 017b, 017c, etc.). It is also known as horizontal partitioning.

By developing a data technology layer, data is partitioned into multiple databases 017, 017a, 017b, 017c, etc. called a shard. This gives an enterprise system more options for scaling and data load distribution. Leveraging the identity service's space definitions is the best option to define and manage shards as part of a system scaling strategy.

It is common for large enterprise systems to interface with various data sources 017 to perform their work. With the emphasis of SOA, internal and external systems now share data more efficiently than ever. Additionally, as enterprise architectures become more understood, the ease of integrating to third-party systems is realized and more SOAs are leveraged when building solutions. As adoption continues, it is important to have a proper architecture that allows for integration to a wide variety of data sources through loose coupling principles.

There are several common data sources 017 where obtaining data occurs through data technology adapter components 060. These include but are not limited to the following.

Relational databases 017a. These are the most common data source for system development. An ORM tool is used by business objects to interface with relational databases. The adapter assists in mapping objects to relational database structure and removes the burden of coding repetitive database CRUD tasks for business objects.

File data sources 017b. Many legacy systems still use files to share data between businesses and systems. Data is exported by one system and imported by another. The adapter reads both ASCII and binary file formats as well as transfers files from common storage locations such as a Content Delivery Network (CDN) share or FTP site.

Blockchain repositories 017c. Emergent blockchain technologies have added a new level of trusted data sharing. They are not relational and require a different mechanism to efficiently manage. The business objects use an OBM logic similar to the ORM logic used with relational databases. An adapter contains this logic and hides the implementation details to interface with blockchain technologies. As in the case with business objects using relational databases, the consumers of business objects are not required to know blockchain technology to complete required business services. Promising blockchain technologies do not replace the need for relational databases but complement them by storing data both on the blockchain and within relational databases.

Socket data sources 017d. Sockets are an inter-process communication mechanism in which a socket is an endpoint of a two-way communication link. An endpoint is a combination of IP (Internet Protocol) address and port number. Sockets allow communication between processes on the same or different machines in diverse environments. An adapter handles all messaging that occurs between processes.

IoT Devices 017e. Communication protocols used for IoT devices vary. Some of the common protocols are Bluetooth, Message Queuing Telemetry Transport (MQTT), Advanced Message Queuing Protocol (AMQP), HyperText Transfer Protocol (HTTP), Constrained Application Protocol (CoAP), Zigbee, Z-Wave, RFID, and NFC. An adapter interface handles messaging that occurs between the IoT device and the system.

Webservices 017. More modern systems that have adopted an SOA publish an integration interface through a webservice. These webservices are implemented using SOAP or REST technologies. An adapter encapsulates this technology and maps the webservice interface to the system.

Legacy systems 017g. Many legacy systems provide integration functionality by a means of user exits. A user exit is a subroutine invoked by a software package for a predefined event in the execution package. Clients of the package substitute their own subroutines in place of the default ones provided by the package vendor to provide customized functionality. In addition, the user exits convert a legacy system into an IoT device where valuable data can now be shared. An adapter bridges and maps the messaging of the legacy system to the protocol used.

Other systems 017h. Other data sources may include quantum systems or other emerging technologies.

Large data-intensive enterprise systems define and organize required data sources 017 using proper object-oriented analysis and design techniques. The most common data source 017 is a relational database 017a. Relational databases 017a are defined and organized through a business object analysis and design process where data and functions are defined. The business objects 015 are then organized into groups of cohesive objects under a common namespace. These groups are used as the logical data model to convert to a physical data model.

A recent and powerful data source 017 to emerge is called blockchain 017c. Blockchain technology is a distributed, decentralized, secure, and trusted ledger. A ledger is a continually evolving list of records or blocks, very similar to a database. These blocks are stored linearly, with each block containing a cryptographic hash of the previous block so that blocks are secure and can never change.

A distributed ledger is shared and synchronized across a network of multiple participants or nodes. A decentralized ledger indicates the same ledger, in its entirety, is located on every node in the network. Distributed ledgers are either public/permissionless or private/permissioned depending on whether anyone (public) or only approved participants (private) can run a node.

Consensus mechanisms. To keep a distributed ledger trusted, a consensus mechanism ensures a majority (or all in some mechanisms) of network participants agree on the validity of data or transactions that are written to the ledger. The consensus mechanism is a set of rules or facts known by network participants and used to keep all nodes in the network on the same page. Common private blockchain consensus algorithms include proof of work, proof of authority, and proof of stake.

SOA is a pattern in computer software design in which application components provide services to other components via a communications protocol, typically over a network. A key advantage of an SOA is it delivers a new level of loosely coupled services by communicating over a network and allows for technology independent service providers.

SOAs provides advantages similar to component-based architectures, such as interoperability and loose coupling to enable high reuse and development efficiency. Components are binaries that have a defined interface. Services use these components to support their interface. Services allow components created with any technology to be accessed over a network.

SOA is a powerful evolution of component-based architectures. It provides the same foundational benefits as component-based architectures and adds the benefits of network and technology independence. When both component-based architectures and SOA concepts are used, a powerful balance occurs that takes advantage of the strengths of both. When architected correctly, SOA is a functional expression of component-based architectures and object-oriented design.

SOA empower components with services that provide external access. This evolves basic component architectures into powerful webservices.

SOAs allow a business tier to be published via a webservice to an internal or external presentation or business tier. This allows business intelligence to be reused across many applications. The publishing occurs by exposing the business components through a webservice interface using RESTful technology. The webservice uses the standard HTTP operations, such as Get, Post, Put, And Delete, along with resource naming to expose appropriate business objects through endpoint operations.

Webservice endpoints map to business object operations. Endpoints are either coarse-grained or fine-grained. Coarse-grained endpoints return more data than fine-grained endpoints but have the advantage of reducing roundtrips to the service for data. By default, endpoints are coarse-grained but can be converted to fine-grained or additional endpoints can be added to make endpoints as fine-grained as necessary.

Business services 014b—business components provide services with business functions to support the service task. Business object components 015—business services use business object components and/or data technology components. Data technology components 016—adapters integrate third-party data sources into the system. Application webservices 014a—webservices integrate to internal or third-party webservices and processes.

FIG. 7 illustrates an embodiment of an authentication process for gaining access to a resource within the system 130. An access token is associated with a scope (set by a resource owner 021) that defines consumer authorization to a webservice. This includes access to only certain endpoints, resources, or functions, such as read/write operations. A validated user has roles within the system that control access to system functions. All identity service entities (API consumer, user, application, and business service) include permissions to spaces. Access to data outside the authorized space is not allowed.

Application and business services must first be authenticated to access system resources through webservices. The application or business service must be known, have a valid access token, and have proper user credentials.

Authentication occurs using the OAuth 2.0 (RFC 6749) standard or other standard that accomplishes the same function. The system may require the grant type of client credentials to be used. When registering with the system, a webservice consumer is given a ClientID and ClientSecret that defines the client credentials. The client credentials request 023 and obtain an access token 024 through an authorization server 027. This access token gives application and business services permission to webservice resources found on the resource server 026.

OAuth 2.0 is an open authentication and access delegation standard leveraged by organizations to manage access through web services. Enterprise organizations may leverage this standard to share information with third-party applications or websites. The identity services 020 function as the OAuth authorization server 027 within this standard.

Applications and/or business services 014 send a token request 023 to the identity service 020 using client credentials. Once the credentials are validated, an access token 024 is given to the consumer. This access token has an expiration to help manage security.

The consumer gains access 025 to the system services found on the resource server 026 with this access token 024. Access to resources will be granted upon access token 024 validation.

Access token 024 issuance through identity services 020 is managed by the resource owner 021 who sets the scope of use 028. Client credentials are validated, and proper scope is applied to the access token 024 to properly secure resources found on the resource server 026.

Receiving permission to access resource on servers 026 includes the steps:
1. Request token (request authorization/authentication) 023;
2. Receive an access token 024 (permission); and
3. Access service or resources that the requester is authorized to access as defined by the scope 028 that is defined be the resource owner 021.

Presentation services take advantage of system intelligence by communicating with the business and data technology component layers. These layers are organized across tiers, or clients to support the needs or capabilities of the technology being chosen for the presentation tier. This layered organization for a presentation client is commonly categorized as thin or rich clients.

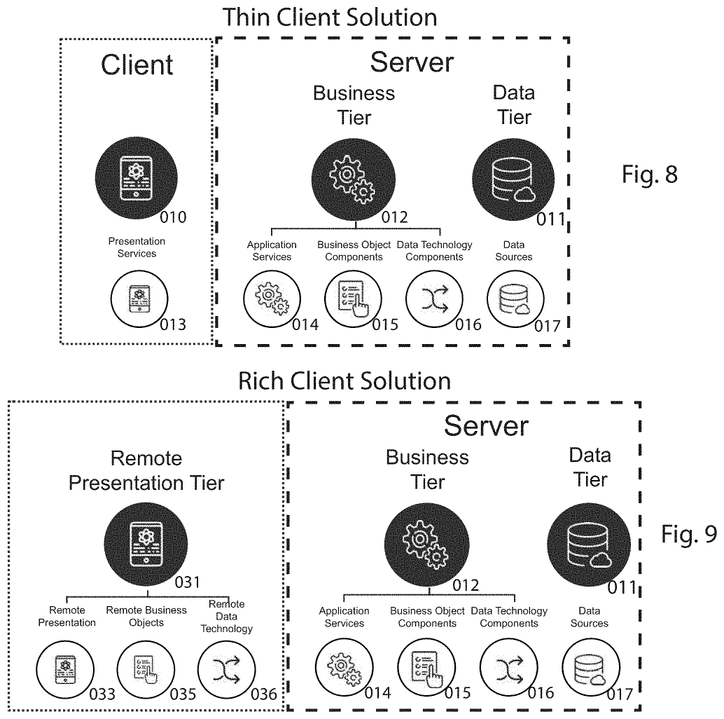

FIG. 8 depicts an organizational embodiment of a system with a thin client solution. Thin clients have business object 015 and data technology component 016 layers residing on the business tier 012. Thin clients rely on the server for all major business and data processing and do very little processing themselves. They mostly deal with presentation responsibilities 010, 013 and only perform very simple data management 016 responsibilities. With a thin client solution, light presentation tiers 031 offer a simple presentation layer consisting of a GUI or non-GUI interface and communicate directly with the business tier webservice 014a.

FIG. 9 depicts an organizational embodiment of system 130 with a rich client solution. Rich clients have business object 015 and data technology component 016 layers residing on the presentation tier 010 or remote presentation tier 031. This provides a more robust set of capabilities within the client. Rich clients have periodic connections to the business tier 012 for system processing but perform many functions without communication to the business tier.

Physical environments are modeled digitally to properly obtain contextual details of a presentation tier 010, 031 device. These are spaces and describe the details of a system location. Digital representations of devices and people are then modeled and associated with these spaces. This keeps people, device, and space information connected to the real-world. Obtaining proper device information on mobile phones, remote client devices, and datacenter servers create a powerful model to obtain insights on how people, devices, and spaces are used.

Enterprise systems have many GUI applications that support the functions of a business. When developing GUI applications in an enterprise environment it is valuable to apply object-oriented concepts for the components that make up the presented views. Understanding the visual needs across applications assists in the development of reusable component. This makes the user experience consistent across the enterprise suite of applications and maximizes code reuse. These reusable components are built to reflect the power of the underlying business components through webservices.

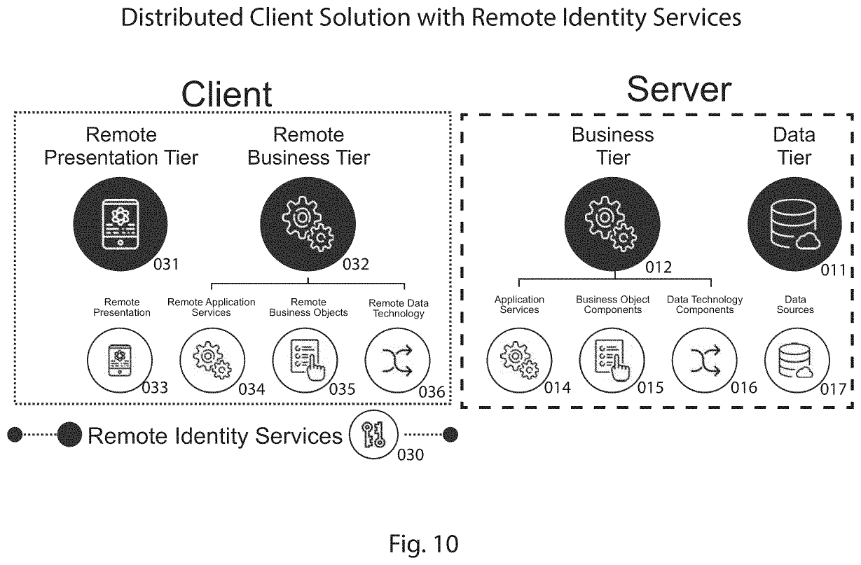

FIG. 10 depicts organizational layers of an embodiment of the system 130. Some remote presentation tier devices have performance, capacity, or security constraints that may not allow for a rich client solution. Additionally, a server business tier may become overwhelmed using a thin client solution. In these cases, an additional remote business tier is added to distribute rich client functionality across two remote tiers.

Advanced enterprise solutions require distributed or remote client services to facilitate more complex client needs. In a DEA, functionality in the form of layers and components are separated on different networked computers. These components communicate over the network using various forms of messaging protocols. Distributing processing costs across computers and multiple business tiers reduce the risk of system downtime and improve efficiency and performance.

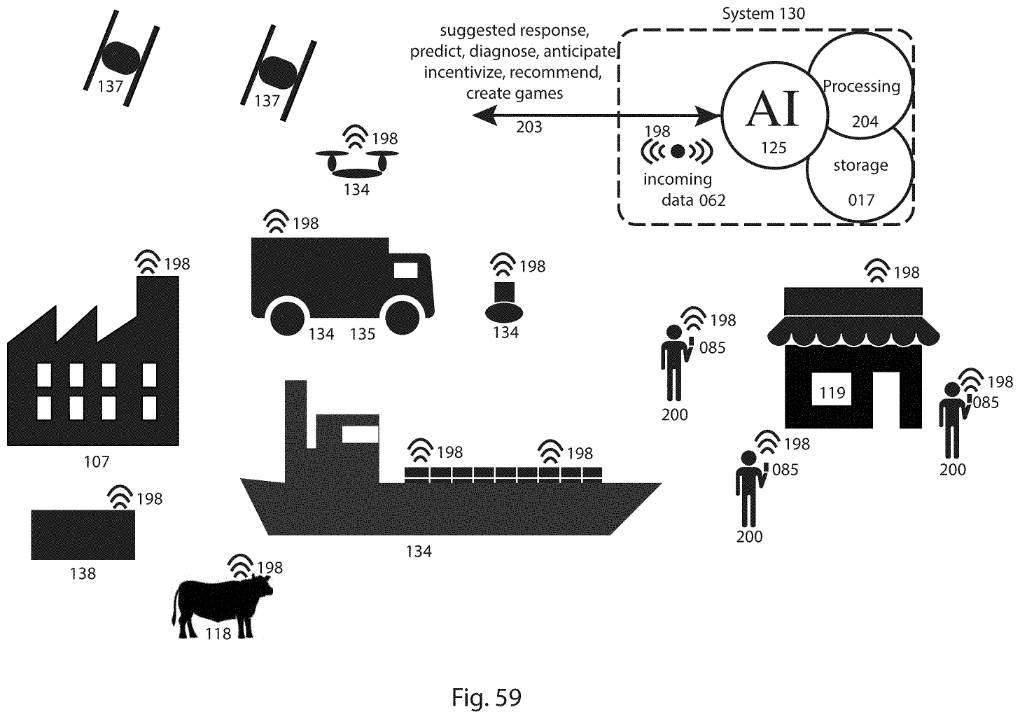

Remote client services use the same N-tier principles of non-distributed solutions. The number of tiers used is dependent on solution complexity. Often in a distributed architecture, the remote presentation tier includes IoT devices 017e, scanners, RFID readers, beacons, sensors of all types, interfaces, input devices of all types and configurations 198 (FIG. 59) and legacy systems 017g. The layout of devices and remote location requirements will affect the number of machines and tiers required. It is common to have both a remote presentation tier 031 and remote business tier 032 located at remote client locations.

Complex enterprise systems require layer distribution to better manage server load and security. A client establishes remote presentation 031 and remote business tiers 032 to physically house these layers on premises. Due to the complex nature of client needs, layers are distributed in different ways but the principle of locally replicating some N-Tier layers remains consistent. These then become remote clients and represent any client that manages some application processes or functions within their physical location.

Remote Representation of N-Tier Layers.

Remote tiers utilize the same layers from a server but use unique implementation methods. This broader distribution of layers leverages a lighter version of their server counterpart and are made complete by their ability to communicate with server layers at the appropriate time. This allows the flexibility to distribute the completeness of layers and balances the needs of the system with the constraints of the client infrastructure.

Server Tiers and Layers.

The server tiers and layers remain consistent when adding remote tiers for a client. The decoupling of layers within the server allows for multiple remote clients to be managed by a relatively unchanged business and data tier. This significantly assists in scaling across various business needs.

Remote Client Layers.

Remote identity services control remote access.

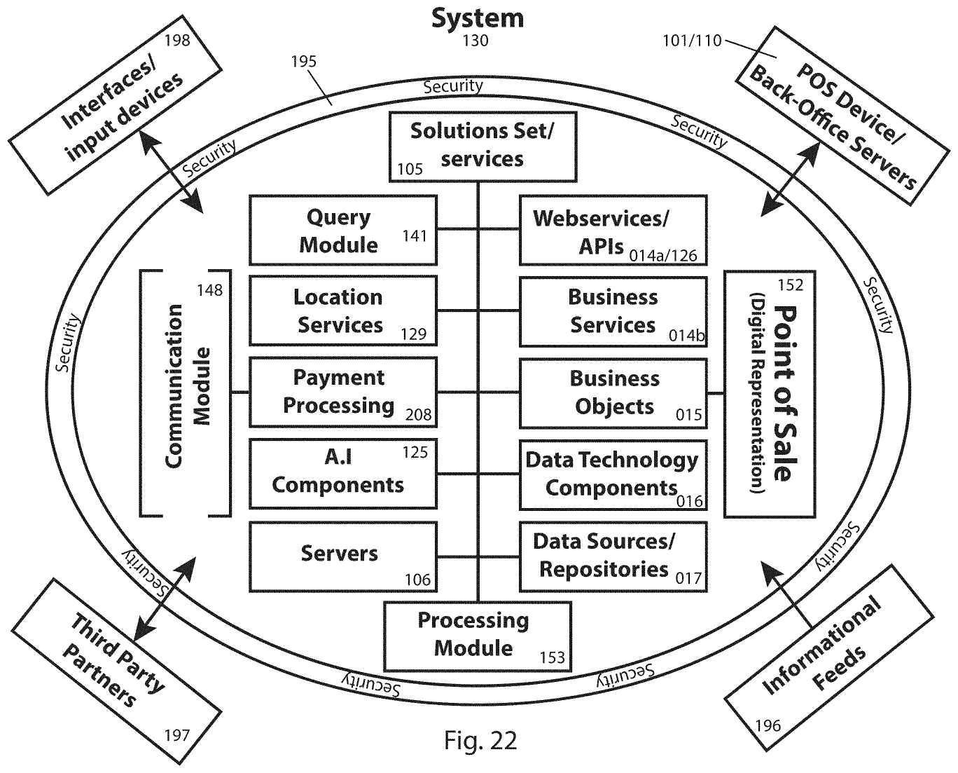

Authentication and authorization of all third-party 197 (FIG. 22) access to a service is critical to system security. An architecture that leverages remote tiers manages access through remote identity services 030.

Remote presentation services 033 are responsible for integrating system functionality to both graphical and non-graphical presentation devices. Multiple presentation devices may exist for clients and all must be managed remotely as part of the system 130. The option for a thin or rich client presentation tier allows remote presentation services to distribute work as required by the presentation device.

Remote business services 034 transform legacy IoT devices to communicate in a modern world.

Complex clients require remote business services to manage client business processes. This balances the need for server resources with the use of client resources. The remote business service intelligence will include the processing and caching of data to optimize the client experience and release the server from unnecessary processing.

Remote business object components 035 manage distributed objects and data communication.

These lighter versions of their server business object component counterpart allow for system intelligence and processing to be distributed based on system needs and client constraints. They leverage the remote data technology components 036 to communicate with the server business tier 012 to optimize and manage the need for server data and processing.

Remote data technology components 036 accessing data remotely.

Remote tiers 032 leverage remote data technology component 036 adapters to communicate with necessary data sources 017. The most commonly used are the webservice 014a and socket adapters but may vary depending on solution complexity. These components do not differ from data technology components 016 within the server business tier, but rather are ones commonly used for remote communication.

Remote applications, IoT devices 017e, legacy systems 017g, and business services 014b within a DEA manage complex interactions between people, places, and things. These interactions require authenticated and authorized connections as well as robust spatial intelligence to give context to operations. This service contains information on users, user roles, access token information, webservice authorization, resource server details, application and business service information, and physical space definitions for remote devices. This information is made available to remote presentation 031 and business tier 032 layers for proper execution of their services.

Remote Security Management.

To properly secure communication within a Distributed Enterprise Architecture, the remote presentation 033 devices, business tier 032 devices, applications, business services, API client credentials, and remote users must all authenticate successfully. Once authenticated, transactions from devices are authorized. Authenticated remote transactions contain powerful contextual information on how devices, users, spaces, and applications and services are used.

Webservice Access.

In a DEA, webservice 014a access is required from the remote tiers 031-032 Remote business object components 035 communicate with a server business tier 012 webservice by first obtaining an access token 024 from the business tier authorization server. Business tier webservices 014a use the OAuth 2.0 standard (or other secure standard) where client credentials obtain an access token 024. Once the access token 024 is received, it is authorized for transactions once the remaining client information successfully authenticates.

Spaces and Services.

To obtain proper contextual details of a remote presentation tier 031 device, the remote physical environment is modeled digitally (digital representations of physical space). These are called spaces. Spaces are details that describe a remote client location. Digital representations of devices are then modeled and associated with these spaces. This keeps device and space information connected to the real world creating a powerful model for authentication and allows the system to obtain insights on how people, devices, and spaces interact.

Device and Spaces.

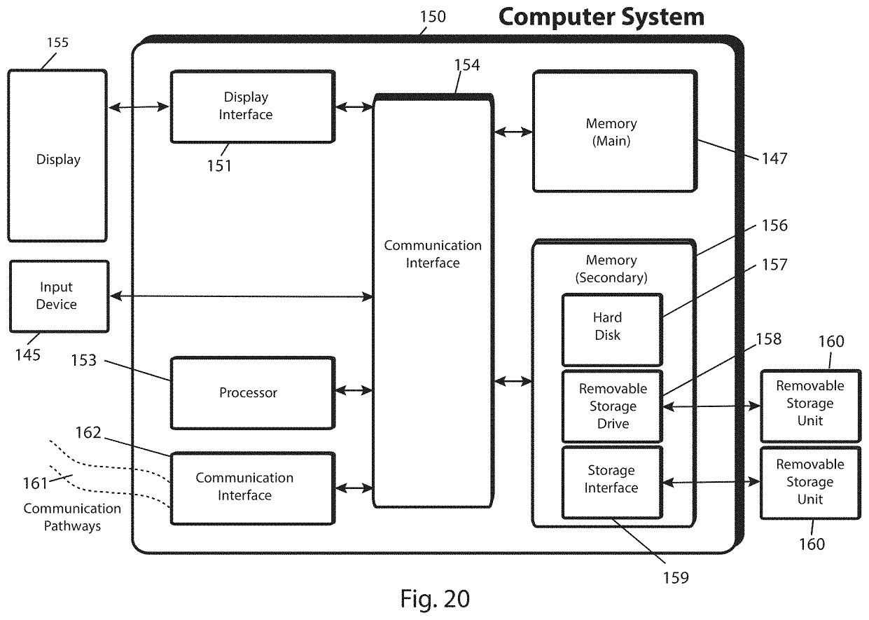

Creating and assigning digital representations can be key to modeling spaces and devices. Modeling space and device information allows the remote identity service to manage the remote client location. Devices and their association with spaces are authenticated and are authorized for activity. Some of the devices include remote computers 150 (FIG. 20), servers 106, legacy systems 017g, interfaces, inputs of all types 198 (FIG. 59), and IoT devices 017e that perform system functions. These devices only run within authorized spaces.

Applications and Business Services, Devices, and Spaces.

Equally important is ensuring that remote services are authorized to execute on devices within a space. Remote applications and business services 034 will not be authorized to perform transactions without the combination of these three elements successfully authenticating against each other.

Remote Users.

In cases where users interact with legacy systems 017g, IoT devices 017e, or applications they must successfully be authenticated and authorized to use the application on a particular device within a space. Groups of users can easily be allowed to use all devices for a group of spaces. If the system requires tight user security to access applications and devices, the remote identity service will ensure this occurs.

Server Tiers and Layers.

The server tiers and layers remain consistent when adding remote tiers for a client. The decoupling of layers within the server allows for multiple remote clients to be managed by a relatively unchanged business 012 and data 011 tier. This significantly assists in scaling across various business needs.

Remote presentation services 033 are both graphical and non-graphical presentations to system users. They are responsible for giving the user the best experience with the business services 014b behind it.

In a DEA, it is common for multiple presentation devices to be present at remote client locations. It is also common for each of these devices to be responsible for multiple presentation devices—an aggregate presentation device structure. The structure can be a combination of legacy systems 017g and IoT devices 017e such as RFID readers, scanners, beacons, interfaces and input of all types 198 (FIG. 59). The remote presentation service 033 interfaces with data technology component adapters 060 to integrate these devices into the solution.

Device Configuration.

It is important for remote devices to leverage space definitions for authentication and authorization management. This process of remote device management is an important aspect of a DEA. Remote locations may have different types of IoT devices that have various configuration requirements. To manage and control a distributed system, it is important that the device and their configuration settings are properly modeled in the system. Once devices are authenticated and authorized, their configuration settings are established and controlled.

Monitoring and Diagnostics.

In a large network of IoT devices 017e, it is important to understand the health of each device, which allows the service provider to—recognize when devices are out of operation, not operating correctly, or out of date on their software version. Capturing device telemetry data helps find device malfunctions and alerts appropriate parties.

Software Maintenance.

To handle business logic changes and fix bugs in software that interface with IoT devices 017e, it is important to have a procedure to update securely and efficiently in many instances, the remote location IT department may want to control updates.

A remote business tier 032 is common in DEA remote layers within a remote presentation tier 031. Business intelligence distribution is needed to handle these constraints and help it perform at optimal levels.

Many clients are deeply invested in legacy systems 017g but want to be part of the internet of things and SOA. IoT devices 017e are the cornerstone of most transformation strategies and there is a significant opportunity to unlock already embedded infrastructure. Legacy systems 017g were not built natively with optimized IoT protocols and therefore require custom data management and communication to model IoT devices 017e. This requires IoT Hubs built for legacy systems 017g to optimally handle the complexity of these systems.

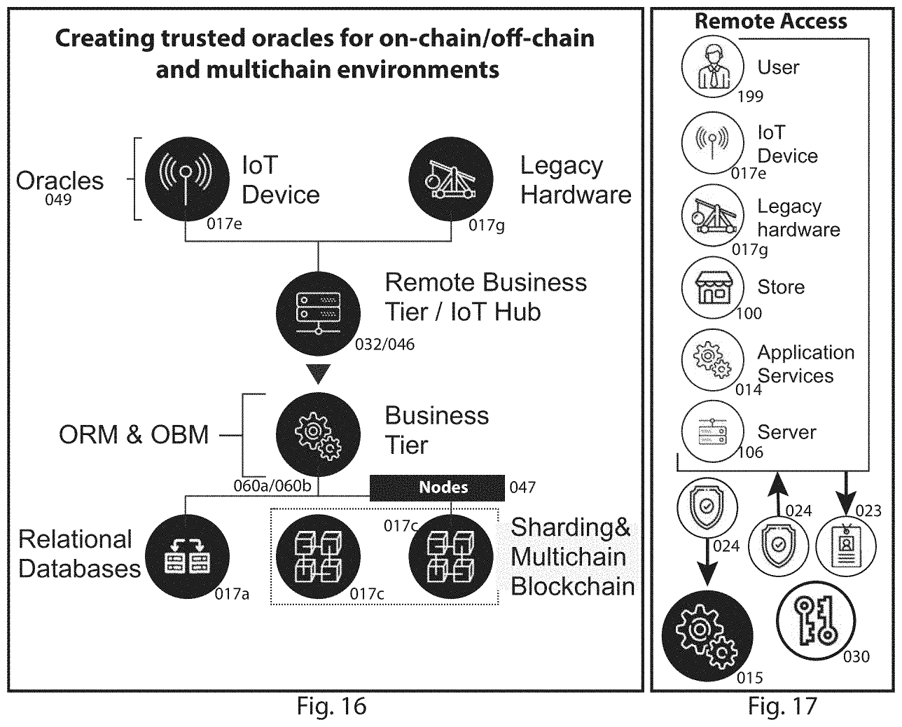

A remote business tier 032 is ideal for an enterprise solution with IoT devices 017e. The business service to manage these devices is called an IoT Hub 046 (FIG. 16). A remote IoT Hub business service can help respond to events, cache data, and execute business rules remotely to help deal with presentation tier or server constraints.

In a DEA, remote business services 034 integrate with cloud 102 services. The remote services are best written with the same business entities used within the cloud services. These business entities are called remote business object components 035 and are replicas of those defined in the server business object components 015.

Remote business object components 035 are lightweight versions of server counterparts. These components contain light validation rules, property caching, and aggregate/child component caching. The execution of larger business processing rules and access to the solution's primary data source occurs through the server business tier using the data technology webservice component adapter.

Supported Environments.

In large DEAs, many devices access services. This includes remote IoT devices 017e, rich client web applications, and mobile applications. Remote business object components 035 exist to support these various client technologies. They offer a loosely coupled business logic layer that allows the presentation layer 033 to focus strictly on differing presentation technologies.

Adapter Communication.

These client components differ from server components because they use remote data technology component adapters 060. Where the server business components 015 use an adapter 060 to a relational database, the remote business components use a webservice adapter 060h to communicate to the business tier webservice 014a. In addition, remote business object components 035 are used within a remote presentation tier 031 to support distributed rich clients. The remote business object components 035 use either a data technology socket adapter 060f or webservice adapter 060h to communicate with the remote business tier 032.

Adapter Object-Webservice Mapping (OWM).

The remote data technology webservice adapter contains similar concepts as the ORM adapter used by business object components mapped to relational databases. The webservice adapter maps remote business object components to the structure required by the business tier webservice. Since the webservice is designed to support the naming and structure of business objects, a common technique to interact with the webservice is used. This technique is called Object-Webservice Mapping (OWM) to reflect the similar concepts used by an ORM.

The OWM is designed to encapsulate the mapping of the standard remote business object CRUD operations to the HTTP verbs Post, Get, Put, And Delete that support RESTful webservices. The business object name is used as the URI resource name and actions become the verb. URIs are formatted as https://<host>/<object name>/{[ObjectID]}/[<action>|<aggregate/child name>].

Webservices connect remote business objects within the remote presentation tier to the primary business object components within the system business tier.

When communicating with a web service, data may be transferred using a JSON object. The object is intelligently defined because it knows which properties are required to be sent, such as an ObjectID or modified properties. Aggregate or child JSON are included when it is required by the operation. In the case of a response containing a JSON object, it is deserialized into its associated business objects.

Remote tiers utilize remote data technology components when managing data and data processes. These components contain the technical implementation to interface with a data source. They do not differ from the data technology components referenced within the server business tier, but rather they are those components more frequently used for remote communication. The most commonly used components are webservices, IoT, files, and legacy system adapters.

The ability to move layers to different tiers allow remote data technology component adapters to be used within both the remote presentation tier and the remote business tier. They contain a powerful encapsulation of technical capability that allows dependent layers to manage the required data and processing.

Presentation Tier Usage.

Remote data technology IoT and legacy system adapters 060d are more commonly used as part of the remote presentation tier 031. They interface with the IoT and legacy system technology to bring valuable data and intelligence to the system 130. The adapters contain the technical implementation to send and receive data.

Business Tier Usage.

Remote business object components 035 can choose to be as light as necessary and use the webservice adapter 060h component to interface with the server business tier 012 at the appropriate time. This distributes the completeness of layers to balance the needs of the system and the constraints of the client infrastructure. The remote business object components 035 will use the OWM capability within the webservice adapter 060h to seamlessly communicate with the server business tier 012.

Other Uses.

The data technology component adapters 060 span a vast number of data sources that support various protocols. The adapters mentioned here and within the server data technology component layer are those that are more commonly used but is not comprehensive and other suitable data technology component adapters 060 types and configurations will be apparent to persons having skill in the relevant art.

Some common remote data technology adapters include:
File adapters 060c—transfer and read/write ASCII or binary files;
Legacy technology adapters 060d—communicate with legacy user exit or similar inerface-based systems;
IoT adapters 060e—communicate with wide range of IoT protocols;
Socket adapters 060f—communicate using fundamental technology on TCP/IP networks;
WebSocket adapters 060h—used as a subject or observer for publication of real-time events;
OWM adapters 060i—map business objects to REST based webservices; and
OBM adapters 060b—Map business object to blockchain network (repository).

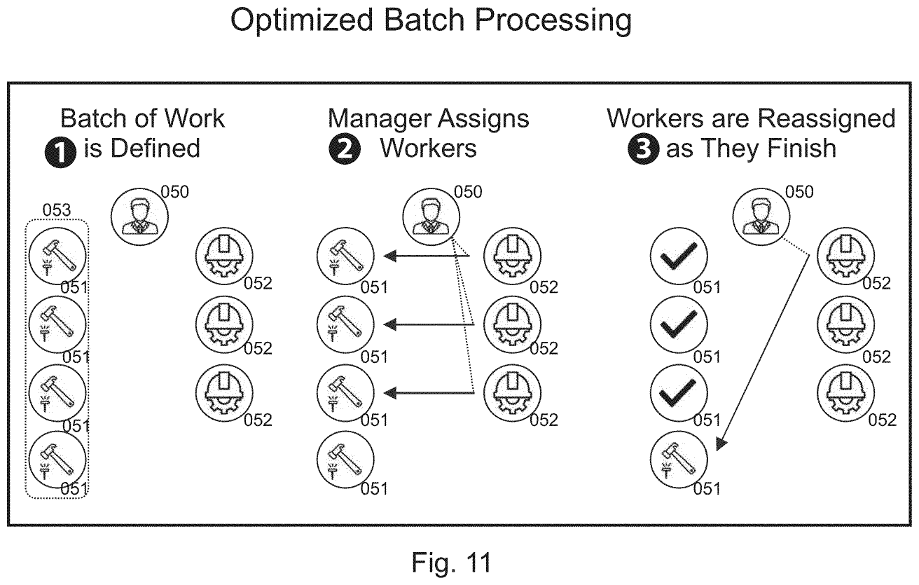

FIG. 11 depicts an example of optimized batch processing within an embodiment of the system. Although batch processing can help tiers manage communication, collecting work together in a single batch can overburden a business service 014b. When a business service 014b is asked to complete more work than it is able, it manages available workers through a batch management process.

A batch manager 050 gathers work into a batch 053. The batch manager knows when and where to find and format work 051. The manager has a predefined number of workers 052 available to process the batch 053. A unit of work 051 within the batch 053 is assigned to an available worker 052. If work within a batch 053 outnumbers the available workers 052, workers 052 are reassigned after completing their unit of work 051.

Managing enterprise workloads. Business services 014b designed for enterprise systems have high throughput requirements. This includes processing large batches 053 of work 051 through complex thread management optimizations. Typical scenarios include processing batches 053 of existing system data, importing data, or interfacing with third-party systems.

When processing large batches 053 of data, whether it be business objects 015 or large datasets returned by a data technology adapter component 016, each unit of work 051 is assigned a worker thread 052. A thread pool manager 050 administers worker threads 052 to ensure optimal use of server resources. Each worker thread 052 has access to existing business object 015 functions, adapters, and other business services 014b to complete its task. Using the power of thread pools and server hardware, business services scale to meet the needs of large enterprise systems.

Business services 014b work with the identity service to determine the spaces it supports. Multiple instances of a business service 014b are created to handle groups of spaces. This allows instances of the business service 014b to be distributed across a server environment to help scale an enterprise system.

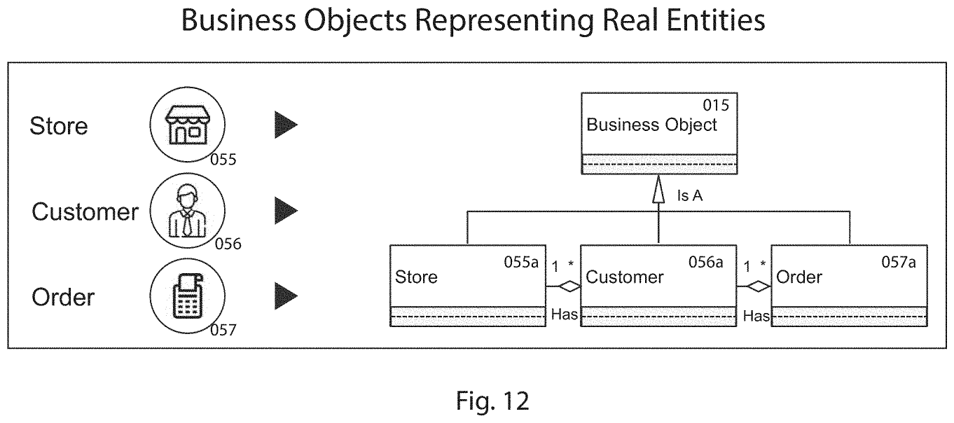

FIG. 12 depicts an embodiment in which business objects represent real entities with the system. Business object components 015 are digital representations of common real-world business entities, locations, devices, etc. These objects contain data and business rules that drive the business. Organizations have real-world entities that are critical participants in their business. These entities have properties, know how to perform functions, have relational order, and have ways in which they interact. Digital components create a business object that represents its real-world equivalent and model relationships between real-world entities. Modeling with the real world as a template help organize increasingly complex enterprise systems. In FIG. 12 the store 055, the customer 056, and the order 057 each have digital representations within the business objects that represent the store 055a, the customer 056a and the order 057a.

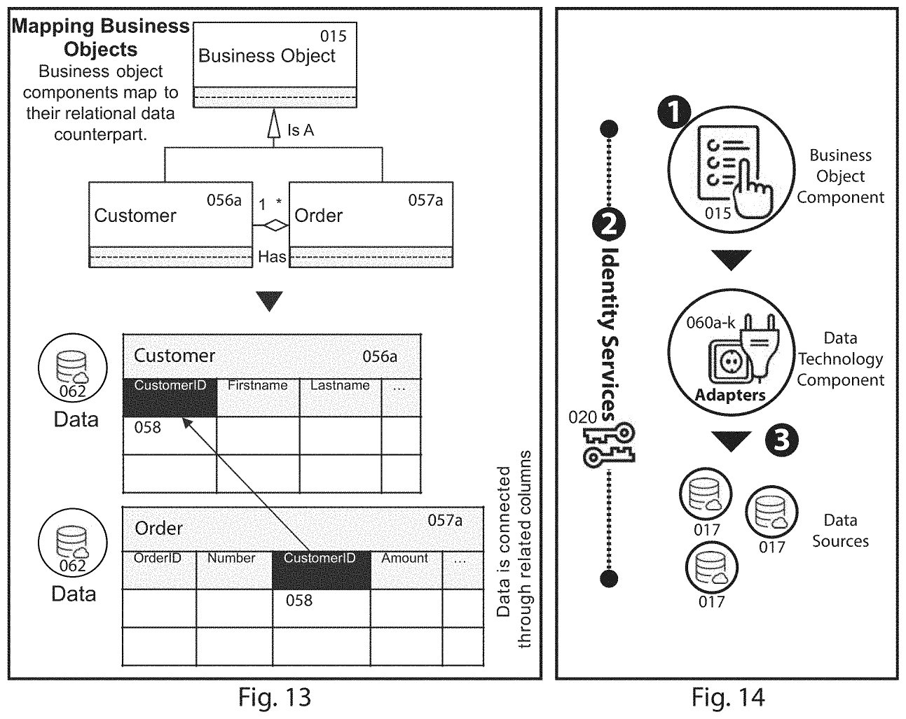

FIG. 13 depicts an instance of mapping business objects within the system. Business object components 015 map to their relational data counterpart. Data is connected through related columns 058.

FIG. 14 depicts a manner of accessing data using adapters within the system. Business object component 015 (or business services 014b in some cases) makes a data request. Identity services 020 help manage complex data organization. Data technology components 016 find and fulfill requests with data sources 017 through an adapter 060.

Data technology adapters 060. An adapter component 060 is used to integrate with data sources 017. It is a powerful software engineering design pattern to allow systems to integrate with one another without changes to the code. This pattern permits the business objects 015 and business services 014b to receive data provided by an adapter 060 and perform actions on the data to keep them loosely coupled with technology. These adapters are built on top of a wide range of technology such as webservices, blockchain, file, or socket technologies. Additionally, these adapters are used remotely when executing a DEA strategy.

There are many types of data technology adapters 060. These include but are not limited to:
ORM adapters 060a—map business objects to relational databases;
OBM adapters 060b—map business objects to blockchain technology;
File adapters 060c—transfer and read/write ASCII or binary files;
Legacy technology adapters 060d—communicate with legacy user exit or similar interface-based systems;
IoT adapters 060e—communicate with wide range of IoT protocols;
Socket adapters 060f—communicate using fundamental technology on TCP/IP networks;
WebSocket adapters 060g—use as a subject or observer for publication of real-time events;
Third-Party webservices adapters 060h—communicate with SOAP or REST based webservices;
OWM adapters 060i—map business objects to REST based webservices;
Other data technology adapters 060j—any adapter or component that helps a technology map a data source, gather data, communicate, etc.; and
POS adapters 060k—loaded by a POS device 101 to handle messages specific to the type of POS 101.

The data technology components 016 use the adapter pattern to interact with data sources 017 using a standard interface. Business objects 015 and business services 014b are the consumers of this data. Business objects 015 use an ORM adapter component 060a to interface with a relational database and an OBM adapter component 060b to interface to a blockchain. Business objects 015 used remotely (remote business objects 035) in a more complex distributed architecture, may use webservice or socket-based adapters 060 depending on the protocol requirements. Business services 014b use adapters 060 of various types and map data into the appropriate business objects 015, 035 as part of their processing. Business services 014b can bypass business objects 015, 035 and talk directly to adapters 060 when integrating third-party data providers.

SOA expands the need for integration. A universal desire to integrate grows as SOA adoption continues. During this process, the ability to deal with legacy forms of data sharing remains important. This requires a technique that keeps data sources 017 loosely coupled to the system. An adapter 060 is a data technology component 016 that maps a data source 017 interface to the interface required by the system and is a powerful design pattern. For enterprise systems, integration may occur with a wide range of data sources 017 that include files 017b, sockets 017d, legacy applications 017g, IoT devices 017e, webservices 017, blockchain technologies 017c, relational database technologies 017a, and other systems 017h.

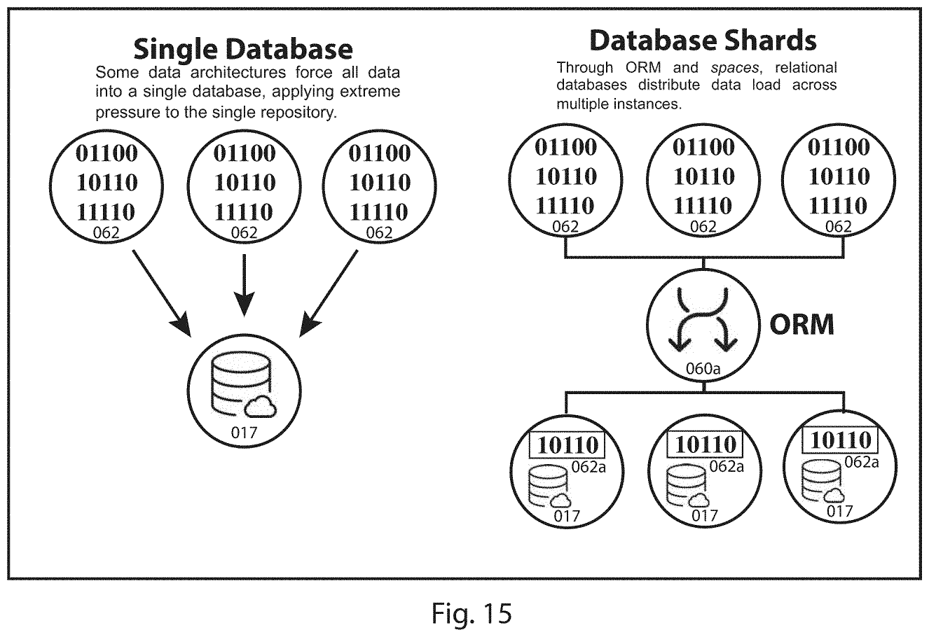

FIG. 15 depicts the difference between a single database, database shards, and blockchain shards. Some data architectures force all data 062 into a single database 017, applying extreme pressure to the single repository. Through ORM adapter 060a and spaces, relational databases 017a distribute data 062 load across multiple instances 062a across potentially multiple database server hardware 017.

Sharding is a horizontal database partition that spreads the data load across multiple instances, or databases, to help achieve scale quickly.

Sharding has become an integral part of most blockchain solutions as it is a perfect solution to manage the large quantiles of data. The system 130 may leverage sharding to achieve horizontal scalability but unlike other blockchain solutions, the system leverages novel sidechains as a core sharding solution. Sidechains are incredibly important to provide data security amongst partners and helps to achieve scalability.

Sidechain sharding breaks a single macro blockchain into several sidechains. This not only helps manage data load but also partitions client data into clear, distinct sub-blockchains. The sidechains still latter up to the same system 130 blockchain 017 so data integrity is not risked.

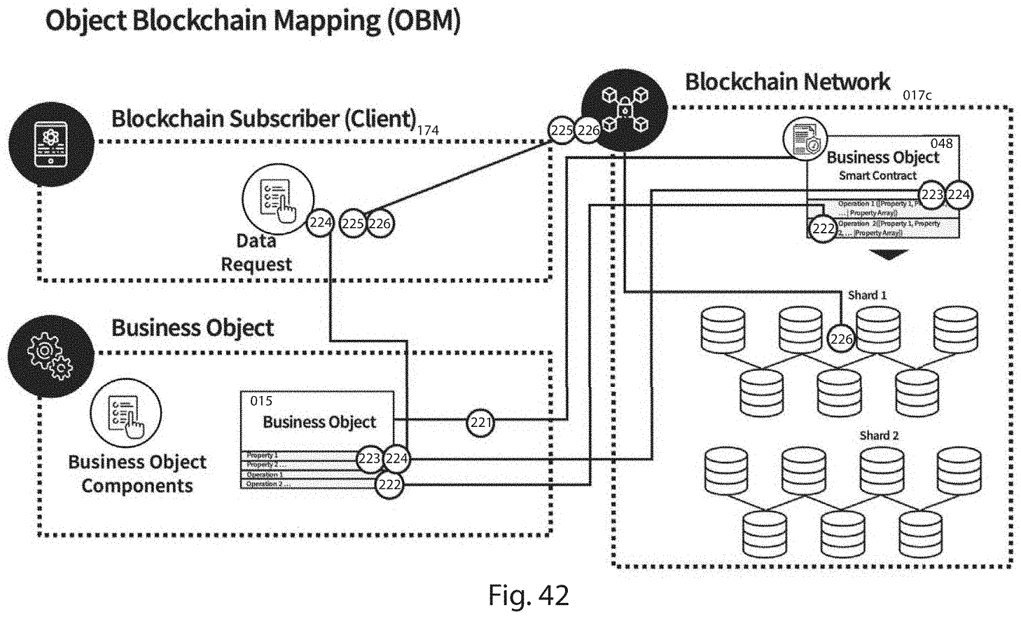

System 130 clients 174 (FIG. 42) and/or third parties 197 (FIG. 22) sit at various levels of data needs and must maintain explicit data permissions to properly secure our client's data. System blockchain sidechains may allow for varying permissions (smart contracts) within the users while also connecting necessary data and validation through the macro blockchain. The blockchain adaptor plug-in will allow the objects to be blockchain aware and allows for interaction with the blockchain. System sharding algorithms inform each object of its destination blockchain based on the transaction being brand, retailer, partner and/or other category.

Each group of objects recognized by its namespace relates to a database instance 062a. In enterprise systems, further analysis is required to determine if a database instance 062a or a segment of its tables will need to be replicated. This is known as database sharding or horizontal partitioning. Each shard is stored within a separate database server instance 062a. A database server instance 062a and its database shards can be placed on separate server hardware 017. This allows for distribution of a systems data 062 over several servers 017, greatly improving performance.

Shard by spaces. Each database shard instance 062a relates to the identity service space definition. By applying space definition to sharding, data 062 partitioning can occur around real-world segmentation. This approach allows enterprise systems to take control of database partitioning and manage it in a way understood by the business. The identity service 020 uses the combination of business object namespace (database instance 062a) and spaces to return the appropriate shard to the ORM data technology component 060a. The ORM 060a then performs necessary data source operations with the shard.

The data technology ORM 060a component manages all database transactions. The ORM 060a database transactions will contain one-to-many database read-write operations. These transactions use ACID (atomicity, consistency, isolation, durability) properties to guarantee the transaction integrity.
Atomicity—All operations succeed or fail as a single unit of work.
Consistency—At transaction completion, the state of data is in a valid state.
Isolation—Transactions unknown by other transactions until complete.
Durability—When transaction completes, data cannot be lost.

Database object naming conventions. Each database instance 062a contains objects such as indexes, tables, triggers, and stored procedures. A standard naming convention is used to remove the difficulty in managing these objects. This naming convention is based on an object-oriented view of database objects: object+[action and/or column]. Table names are defined in the logical data model such as customer and store. Tables added to support the physical data model reflect the relationships they support such as a CustomerStore table. Indexes are prefixed with IDX followed by table name and columns involved in the index such as IDX_Customer_StateCode. Store procedures are named using Table+Action+[By+Columns]. An insert stored procedure for a customer is named CustomerInsert where a stored procedure to retrieve customers by state is named CustomerGetByStateCode.

Database Table Identification.

All non-lookup tables are defined with a sequential GUID as the primary key. The GUID is designed to be sequential to work efficiently with standard database indexing and remove index fragmentation that can occur using standard GUIDs. The GUID uniquely identifies each record and subsequently each business object instance. This has many advantages including the ability to create identifiers outside of the database system, a simplified process of merging shards of like data 062 and used as the object identifier for webservice's RESTful interfaces.

Blockchain sharding. Sharding within the blockchain works very similar to traditional database sharding where scalability, latency, and transaction throughput issues are managed by data segmentation. Blockchain sharding differs in that each node only contains information for that shard and not the entire blockchain. Decentralization is still maintained. Each blockchain shard relates to the identity service space definition allowing blockchain data to be segmented around real-world entities.

FIG. 16 depicts an embodiment of the system showing on-chain/off-chain and multichain environments. One of the most recent and powerful data sources to emerge is called blockchain 017c. Blockchain technology is a distributed, secure, trusted ledger, and can be decentralized. A ledger is a continually evolving list of records or blocks, very similar to a database. These blocks are stored linearly, with each block containing a cryptographic hash of the previous block so that blocks are secure and can never change.

A distributed ledger is shared and synchronized across a network of multiple participants or nodes. A decentralized ledger most likely indicates the same ledger, in its entirety, is located on every node in the network. Distributed ledgers are either public/permissionless or private/permissioned depending on if anyone (public) or only approved participants(private) can run a node.

To keep a distributed ledger trusted, a consensus mechanism ensures a majority (or all in some mechanisms) of network participants agree on the validity of data or transactions that are written to the ledger. The consensus mechanism is a set of rules or facts known by network participants and used to keep all nodes in the network on the same page. Common private blockchain consensus algorithms include proof of work, proof of authority, and proof of stake.

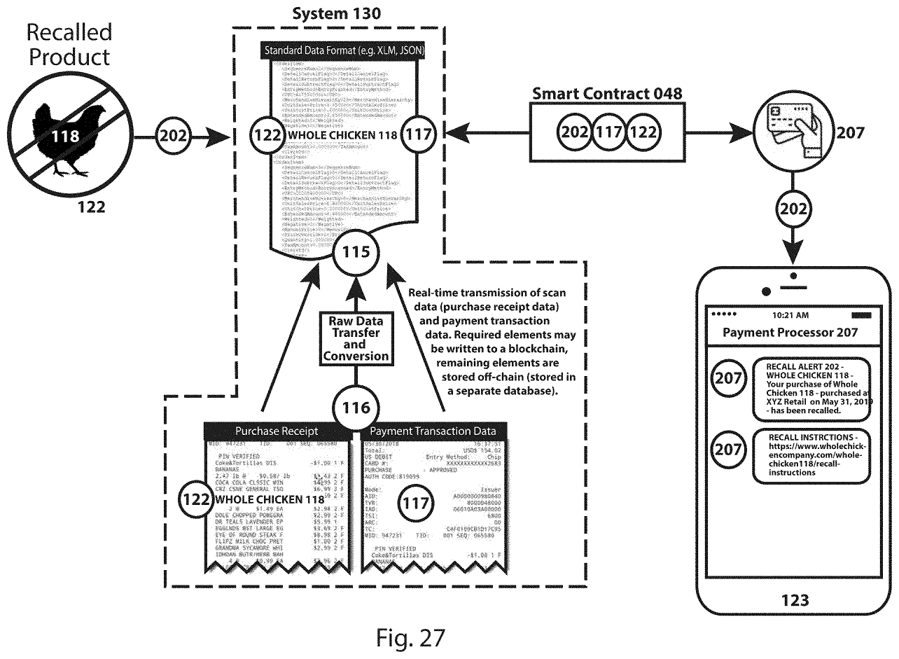

An oracle 049 is a third-party data source 017 that supplies data to the blockchain 017c, through smart contracts 048 (FIG. 27). In FIG. 16 the oracles 049 are an IoT Device 017e and Legacy Hardware 017g (e.g. A legacy retail POS). A smart contract 048 is computer code running on top of a blockchain network and contains a set of rules and conditions to which all participants agree. When executed, incoming data is processed against these rules and conditions. When met, data is accepted to the blockchain 017c. Each participant node is a device that stores a copy of the data 062. Each node stores a smart contract 048 and, therefore, each must execute it and return the same result.

IoT devices 017e are the cornerstone of many transformation strategies and there is a significant opportunity to unlock existing legacy infrastructure. A remote business hub 032/IoT hub 046 converts legacy hardware 017g to an IoT Device 017e. The remote business tier 032 functions as a localized server to collect, control, and batch communication to the primary server. When communicating with IoT devices 017e, the IoT Hub 046 independently manages each device, its events, and its data 062 within a unique IoT business object 015.

Many clients are deeply invested in legacy systems 017g but require data 062 contained within these systems to be unlocked and made available in a trusted, distributed ledger. Legacy systems 017g are converted to IoT devices 017e using a secure and efficient process. Most legacy systems 017g can be converted to an IoT device 017e to unlock new functionality and data sources 017, without a significant hardware investment. Businesses can transform their organization by leveraging a remote business tier 032 to manage this expanded capability through remote business tier 032 communication.

This capability, in union with the distributed ledger functionality of blockchain 017c, allows traditionally hard to access data 062 to now be published to the blockchain 017c. Through a business tier that employs OBM 060b, this difficult-to-access data 062 is now open to be published to multiple independent blockchains 017c, if required. This uncovers new revenue possibilities and awakens transformation change.

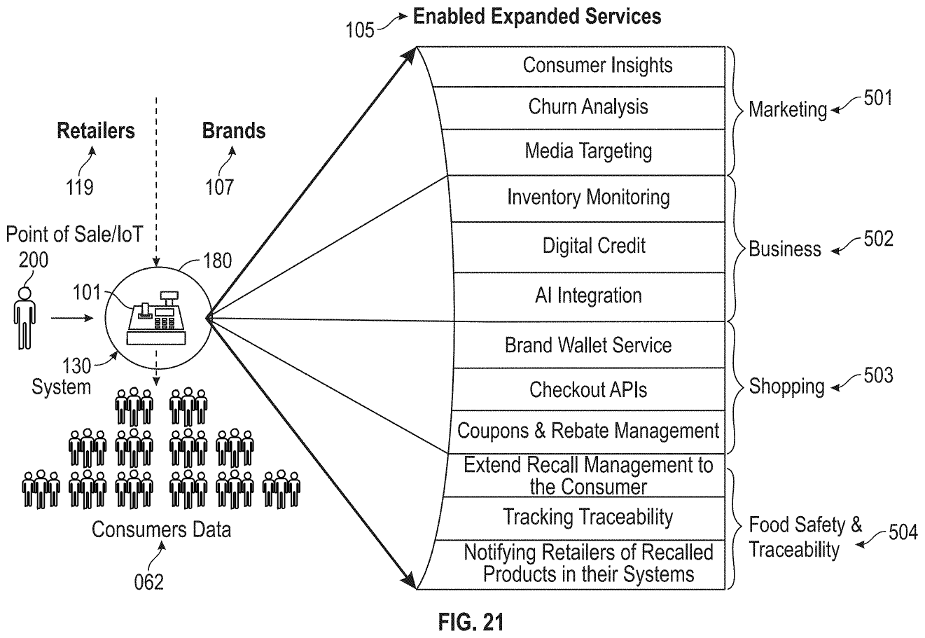

System off-chain transactions are part of a data exchange but will not be placed within the blockchain. Not all data is pertinent to the partners 197 (FIG. 22), retailers 119 (FIG. 21), and brands 107 (FIG. 21). Additionally, many legacy systems 017g are unable to communicate directly with any blockchain database 017c. On-Chain/Off-chain transaction management allows for the blockchain 017c to observe exchanges that occur outside the blockchain (off-chain) and then on-chain the appropriate data 062.

When engaging with blockchain technologies, the system may fulfill certain transactions off-chain and then on-chain them (or select portions of them) to validate and share pertinent data 062a.

FIG. 17 depicts an embodiment of remote access for the system. After a remote identity service authenticates remote tiers 031-032, that remote tier is provided authorized access to business tier services 014b. OAuth 2.0 may provide the best in class industry standards that are implemented for remote authentication and authorization access.

Remote Users.

In cases where users interact with legacy systems 017g, IoT devices 017e, or applications they must successfully be authenticated and authorized to use the application on a particular device within a space. Groups of users can easily be allowed to use all devices for a group of spaces. If the system requires tight user security to access applications and devices, the remote identity service 030 will ensure this occurs.

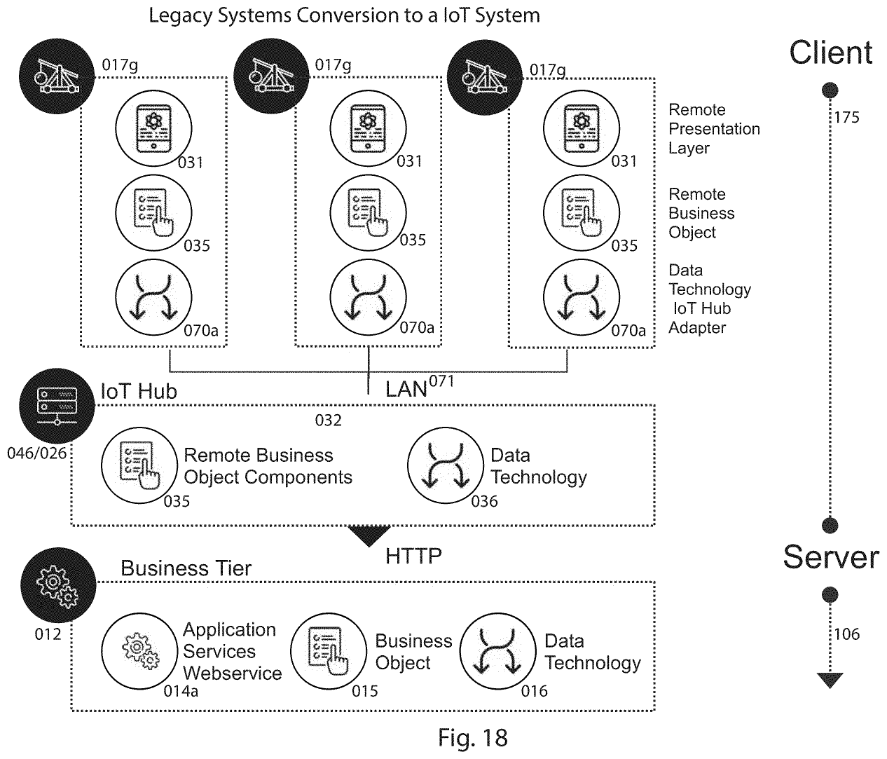

FIG. 18 depicts the conversion of legacy system hardware to an IoT device within the system.

Most legacy systems 017g can be converted to an IoT device 017e to unlock new functionality and data sources, without a significant hardware investment. Businesses can transform their organization by leveraging a remote business tier 032 to manage this expanded capability through remote business tier communication.

Legacy hardware communicates with an IoT Business Hub.

Remote Presentation Tier 031 constraints may dictate using light-weight client business objects 035 with a data technology IoT Hub adapter 070a to communicate to the remote business tier 032 where IoT devices are managed.

The remote presentation tier 031 communicates with IoT hub/server 046/026 over a local area network (LAN). The network may actually be any network suitable for performing the functions as disclosed herein and may include a LAN, a wide area network (WAN), a wireless network (e.g., WiFi), a mobile communication network, a satellite network, the Internet, fiber optic, coaxial cable, infrared, radio frequency (RF), or any combination thereof. Other suitable network types and configurations will be apparent to persons having skill in the relevant art.

The remote business tier 032 functions as a localized server 026 to collect control, and batch communication to the primary server. When communicating with IoT devices, the IoT Hub 046 independently manages each device, its events, and its data within a unique IoT business object.

The business tier 012 communicates to client business objects 035 through a webservice 014a. The webservice constructs business object components 015 using the JSON/XML payload to service the desired action.

Virtually Replicate IoT Devices.

In a remote distributed environment with several IoT devices, the IoT Hub 046 complexity increases. It is important to have digital replicas (digital representations) of IoT devices 017e to help the IoT Hub 046 authenticate and authorize their use and manage device configuration, data, and events. These digital replicas are components inside the remote business object layer 032 and managed by the IoT Hub/server 046/026. The high volume of simultaneous events and data is managed to ensure no data and event loss occurs across devices.

These components capture events and data that are used for local processing and permits client activity to remain at the remote location until it is necessary to submit to the server business tier 012. Communication remains local between the remote presentation tier 031 and the remote business tier 032 to help deal with constraints at the remote location.

Legacy system IoT devices (017g converted to 017e) become business object components 015 by modeling physical IoT devices 017e in digital form. These business objects 015 communicate with blockchain technologies using a data technology OBM adapter component 060b. The OBM 060b allows business objects 015 to be loosely coupled to blockchain 017c technologies. This component works in similar fashion as the ORM adapter component 060a where business object properties and aggregate/child information are shared with the blockchain 017c and validated through smart contracts 048. When business object components 015 work with both the OBM 060b and ORM 060a adapters, management of on-chain and off-chain data 062 is seamless and powerful. With the current latency in many blockchain technologies, it is not reasonable to write all data to a blockchain. Some data is therefore kept off-chain (not written to the blockchain) and some is written on-chain (on the blockchain). The OBM 060b is also a powerful mechanism that enables blockchain interoperability, the writing to multiple independent blockchain networks, or multichain.

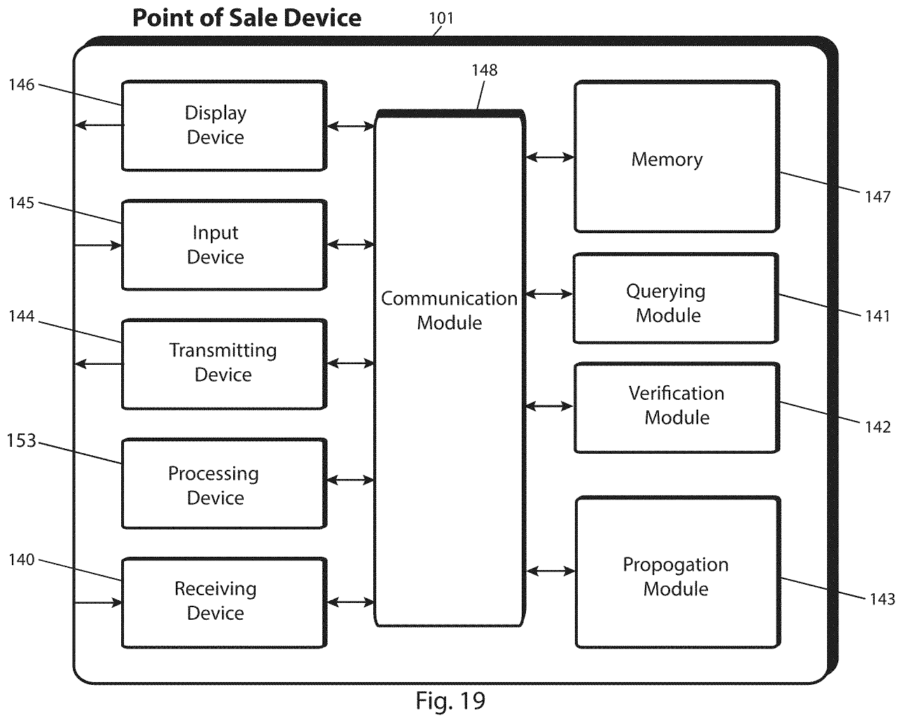

FIG. 19 illustrates an embodiment of a POS device 101 in the system 130. It will be apparent to persons having skill in the relevant art that the embodiment of the POS device 101 as shown in FIG. 19 is provided as illustration only and may not be exhaustive to all possible configurations of the POS device 101 suitable for performing the functions as discussed herein. For example, the computer system 150 illustrated in FIG. 20 and discussed in more detail below may be a suitable configuration of the POS device 101.

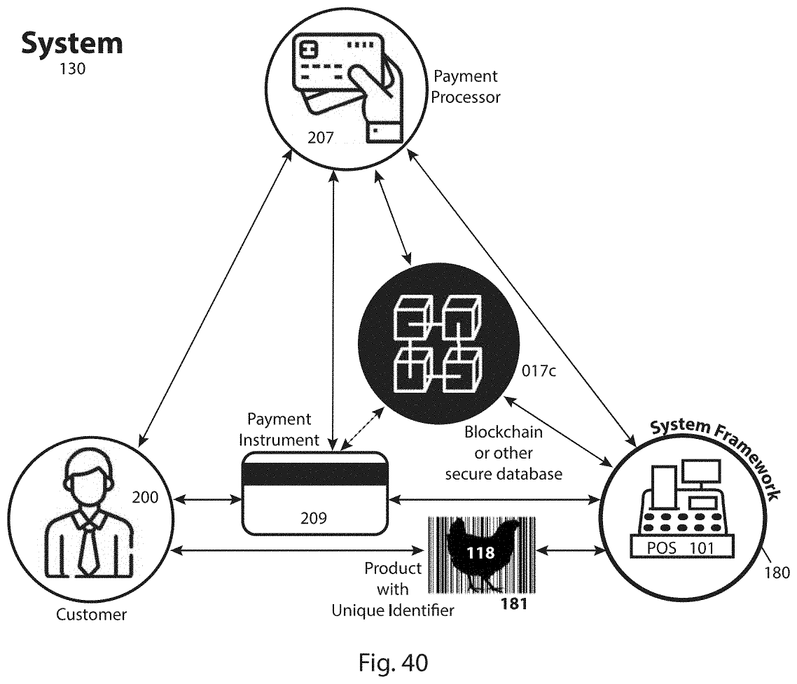

The POS device 101 may include or be otherwise interfaced with one or more input devices 145. The input devices 145 may be internal to the POS device 101 or external to the POS device 101 and connected thereto via one or more connections (e.g., wired or wireless) for the transmission of data and/or information to and/or from. The input devices 145 may be configured to receive input from a user of the POS device 101 which may be provided to another module or engine of the POS device 101 (e.g., via the communication module 148) for processing accordingly. Input devices 145 may include any type of input device suitable for receiving input for the performing of the functions discussed herein, such as a keyboard, mouse, click wheel, scroll wheel, microphone, touch screen, track pad, scanner, chip reader, magnetic strip reader, camera, optical imager, etc. The input device 145 may be configured to, for example, scan of bar codes, QR codes or other types of machine readable code, read data encoded in a magnetic stripe of a payment instrument 209 (FIG. 40), read a machine-readable code displayed by a payment instrument 209 and decode data encoded therein, or receive data input by a communication device and/or an individual or customer 200, where such data may include payment credentials and/or data associated with an identification value stored in a transaction in the blockchain.

The POS device 101 may also include a processing device 153. The processing device 153 may be configured to perform the functions of the POS device 101 discussed within this disclosure as will be apparent to persons having skill in the relevant art. In some embodiments, the processing device 153 may include and/or be comprised of a plurality of engines and/or modules specially configured to perform one or more functions of the processing device 153, such as a querying module 141, verification module 142, generation module 143, etc. (even though they might be depicted as separate elements in the figure). As used herein, the term “module” may be software or hardware particularly programmed to receive an input, perform one or more processes using the input, and provides an output. The input, output, and processes performed by various modules will be apparent to one skilled in the art based upon the present disclosure.

The POS device 101 may also include a communication module 148. The communication module 148 may be configured to transmit data between modules, engines, databases, memories, and other components of the POS device 101 for use in performing the functions discussed in this disclosure. The communication module 148 may be comprised of one or more communication types and utilize various communication methods for communications within a computing device. For example, the communication module 148 may be comprised of a bus, contact pin connectors, wires, etc. In some embodiments, the communication module 148 may also be configured to communicate between internal components of the POS device 101 and external components of the POS device 101, such as externally connected databases, display devices, input devices, servers, etc.

The POS device 101 may include a receiving device 140. The receiving device 140 may be comprised of multiple devices, such as different receiving devices for receiving data over different networks, such as a first receiving device for receiving data over a LAN and a second receiving device for receiving data via the Internet. The receiving device 140 may be configured to receive data from payment instruments 209, blockchain networks/repositories 017c, the system 130, third-party partners 197 (FIG. 22), payment processors 207, and other systems and entities via one or more communication methods, such as NFC, physical contact points, WiFi, Bluetooth, LAN, cellular communication networks, the Internet, etc. The receiving device 140 may be configured to receive data over one or more networks via one or more network protocols. The receiving device 140 may receive electronically transmitted data signals, where data may be superimposed or otherwise encoded on the data signal and decoded, parsed, read, or otherwise obtained via receipt of the data signal by the receiving device 140. In some instances, the receiving device 140 may include a parsing module for parsing the received data signal to obtain the data superimposed thereon. For example, the receiving device 140 may include a parser program configured to receive and transform the received data signal into usable input for the functions performed by the processing device to carry out the methods and systems described herein.

The receiving device 140 may also be configured to receive data signals electronically transmitted by payment processors 207, which may be superimposed or otherwise encoded with notifications indicating approval or denial of electronic payment transactions. The receiving device 140 may be further configured to receive data signals electronically transmitted by payment instruments 209, which may be superimposed or otherwise encoded with digital signatures, public keys, blockchain addresses, or other data used in the identification and authentication of stored in the blockchain and the posting of new wallet, promotion, incentive, consumer safety data thereto.

The POS device 101 may also include a memory 147. The memory 147 may be configured to store data for use by the POS device 101 in performing the functions discussed herein, such as public and private keys, symmetric keys, etc. The memory 147 may be configured to store data using suitable data formatting methods and schema and may be any suitable type of memory, such as read-only memory, random access memory, etc. The memory 147 may include, for example, encryption keys and algorithms, communication protocols and standards, data formatting standards and protocols, program code for modules and application programs of the processing device, and other data that may be suitable for use by the POS device 101 in the performance of the functions disclosed herein as will be apparent to persons having skill in the relevant art. In some embodiments, the memory 147 may be comprised of or may otherwise include a relational database that utilizes structured query language for the storage, identification, modifying, updating, accessing, etc. of structured data sets stored therein. The memory 147 may be configured, for example, to store blockchain data, digital signatures, private keys, public keys, and other data used as discussed in this disclosure

The POS device 101 may include or be otherwise interfaced with a display device 146. The display device 146 may be internal to the POS device 101 or external to the POS device 101 and connected thereto via one or more connections (e.g., wired or wireless) for the transmission of data to and/or from. The display device 146 may be configured to display data to a user of the POS device 101. The display device 146 may be any type of display suitable for displaying data as part of the functions discussed herein, such as a liquid crystal display, light emitting diode display, thin film transistor display, capacitive touch display, cathode ray tube display, light projection display, head mount display, etc. In some instances, the POS device 101 may include multiple display devices 146. The display device 146 may be configured to, for example, display data associated with an electronic payment transaction, such as a transaction amount, processing status, approval status, etc.

The POS device 101 may include a querying module 141. The querying module 141 may be configured to execute queries on databases to identify information. The querying module 141 may receive one or more data values or query strings and may execute a query string based thereon on an indicated database, such as the memory, to identify information stored therein. The querying module 141 may then output the identified information to an appropriate engine or module of the POS device 101 as necessary. The querying module 141 may, for example, execute a query on the memory 147 to identify transaction values included in blocks received from the blockchain network/repository 017c

The POS device 101 may also include a verification module 142. The verification module 142 may be configured to verify data as part of the functions of the POS device 101 as discussed herein. The verification module142 may receive data to be verified as input, may attempt to verify the data, and may output a result of the attempted verification to another module or engine of the POS device 101. The verification module 142 may, for example, verify digital signatures that are included in transaction values identified in the blockchain using public keys received (e.g., by the receiving device 140) from payment instruments 209. The verification module 142 may also be configured to verify eligibility of a promotion or incentives for redemption based on blockchain data, such as to verify that a promotion or incentive was not previously redeemed and has not been transferred to a null address or an address associated with invalidation.

The POS device 101 may also include a propagation module 143. The propagation module 143 may be configured to propagate data as part of the functions of the POS device 101 as discussed herein. The propagation module 143 may receive an instruction as input, may propagate data based on the instruction, and may output the propagated data to another module or engine of the POS device 101. The propagation module 143 may, for example, be configured to generate blockchain transaction values, such as may include a digital wallet, promotion, incentive and/or consumer safety identification value and the digital wallet, promotion, incentive and/or consumer safety data. Digital wallet, promotion, incentive, and consumer safety data may include, for instance, transaction number, store number, POS number, transaction date, a transaction modifier, expiration date, start date, redemption limit, etc. The propagation module 143 may also be configured to generate key pairs and/or digital signatures, as applicable, for the performing of the functions discussed in this disclosure.

The POS device 101 may also include a transmitting device 144. The transmitting device 144 may be configured to transmit data over one or more networks via one or more network protocols. In some instances, the transmitting device 144 may be configured to transmit data to blockchain networks, payment instruments 209, payment processors 207, and other entities via one or more communication methods, such as NFC, physical contact points, Bluetooth, radio frequency, LAN, cellular communication networks, the Internet, etc. In some embodiments, the transmitting device 144 may be comprised of multiple devices, such as different transmitting devices for transmitting data over different networks, such as a first transmitting device for transmitting data over a LAN and a second transmitting device for transmitting data via the Internet. The transmitting device 144 may electronically transmit data signals that have data superimposed that may be parsed by a receiving computing device. In some instances, the transmitting device 144 may include one or more modules for superimposing, encoding, or otherwise formatting data into data signals suitable for transmission.

The transmitting device 144 may be configured to electronically transmit data signals to blockchain networks that are superimposed or otherwise encoded with transaction values, which may include wallet, promotion, incentive and/or consumer safety identification values and wallet, promotion, incentive and/or consumer safety data. The transmitting device 144 may also electronically transmit data signals to blockchain networks that are superimposed or otherwise encoded with data requests, such as to request new blocks or transaction values for review or use thereof by the POS device 101. The transmitting device 144 may also be configured to electronically transmit data signals to payment instruments 209, such as may be superimposed or otherwise encoded with private keys, public keys, digital signatures, blockchain addresses, or other data necessary for use by the payment instrument 209 to authentication access thereof to a wallet, promotion, incentive and/or consumer safety data. The transmitting device 144 may be further configured to electronically transmit data signals to payment processors 207 and/or a payment network such as may be superimposed or otherwise encoded with transaction data for the processing of an electronic payment transaction, such as payment credentials, a transaction amount, geographic location, store number, POS number, transaction number, time and/or date, currency type, product data, merchant data, credit data, debit data, acquirer data, issuer data, incentive data, reward data, loyalty data, offer data, digital wallet data, promotion data, incentive and/or consumer safety data, etc.

FIG. 20 depicts a computer system 150 in which embodiments of the present disclosure, or portions thereof, may be implemented as computer-readable code. For example, the POS device 101 of FIG. 19 may be implemented in the computer system 150 using hardware, software, firmware, non-transitory computer readable media having instructions stored thereon, or a combination thereof and may be implemented in one or more computer systems or other processing systems. Hardware, software, or any combination thereof may embody modules and components used to implement the methods and systems the objects of the invention.

If programmable logic is used, such logic may execute on a commercially available processing platform configured by executable software code to become a specific purpose computer or a special purpose device (e.g., programmable logic array, application-specific integrated circuit, etc.). A person having ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device. For instance, at least one processor device and a memory may be used to implement the described embodiments herein.

Processor device 153 may be a special purpose or a general-purpose processor device specifically configured to perform the functions discussed herein. The processor device 153 may be connected to a communications infrastructure 154, such as a bus, message queue, network, multi-core message-passing scheme, etc. The network may be any network suitable for performing the functions as disclosed herein and may include a local area network (LAN), a wide area network (WAN), a wireless network (WiFi), a mobile communication network, a satellite network, the Internet, fiber optic, coaxial cable, infrared, radio frequency (RF), or any combination thereof. Other suitable network types and configurations will be apparent to persons having skill in the relevant art.

The computer system 150 may also include a main memory 147 (e.g., random access memory, read-only memory, etc.), and may also include a secondary memory 156. The secondary memory 156 may include the hard disk drive 157 and a removable storage drive 158, such as a floppy disk drive, a magnetic tape drive, an optical disk drive, a flash memory, etc.

The removable storage drive 158 may read from and/or write to the removable storage unit 160 in a well-known manner. The removable storage unit 160 may include a removable storage media that may be read by and written to by the removable storage drive 158. For example, if the removable storage drive 158 is a floppy disk drive or universal serial bus port, the removable storage unit 160 may be a floppy disk or portable flash drive, respectively. In one embodiment, the removable storage unit 160 may be non-transitory computer readable recording media.

In some embodiments, the secondary memory 156 may include alternative means for allowing computer programs or other instructions to be loaded into the computer system 150, for example, the removable storage unit 160 and an interface 159. Examples of such means may include a program cartridge and cartridge interface (e.g., as found in video game systems), a removable memory chip (e.g., EEPROM, PROM, etc.) and associated socket, and other removable storage units 160 and interfaces 159 as will be apparent to persons having skill in the relevant art.

Data stored in the computer system 150 (e.g., in the main memory 147 and/or the secondary memory 156) may be stored on any type of suitable computer readable media, such as optical storage (e.g., a compact disc, digital versatile disc, Blu-ray disc, etc.) or magnetic tape storage (e.g., a hard disk drive). The data may be configured in any type of suitable database configuration, such as a relational database, a structured query language (SQL) database, a distributed database, an object database, etc. Suitable configurations and storage types will be apparent to persons having skill in the relevant art.

The computer system 150 may also include a communications interface 162. The communications interface 162 may be configured to allow software and data to be transferred between the computer system 150 and external devices. Exemplary communications interfaces 162 may include a modem, a network interface (e.g., an Ethernet card), a communications port, a PCMCIA slot and card, etc. Software and data transferred via the communications interface 162 may be in the form of signals, which may be electronic, electromagnetic, optical, or other signals as will be apparent to persons having skill in the relevant art. The signals may travel via a communications path 161, which may be configured to carry the signals and may be implemented using wire, cable, fiber optics, a phone line, a cellular phone link, a radio frequency link, etc.

The computer system 150 may further include a display interface 151. The display interface 151 may be configured to allow data to be transferred between the computer system 150 and external display 155. Exemplary display interfaces 151 may include high-definition multimedia interface (HDMI), digital visual interface (DVI), video graphics array (VGA), etc. The display 155 may be any suitable type of display for displaying data transmitted via the display interface 151 of the computer system 150, including a cathode ray tube (CRT) display, liquid crystal display (LCD), light-emitting diode (LED) display, capacitive touch display, thin-film transistor (TFT) display, etc.

Computer program medium and computer usable medium may refer to memories, such as the main memory 147 and secondary memory 156, which may be memory semiconductors (e.g., DRAMs, etc.). These computer program products may be means for providing software to the computer system 150. Computer programs (e.g., computer control logic) may be stored in the main memory 147 and/or the secondary memory 156. Computer programs may also be received via the communications interface 162. Such computer programs, when executed, may enable computer system 150 to implement the present methods as discussed herein. In particular, the computer programs, when executed, may enable processor device 153 to implement the methods illustrated in the figures and as discussed in this disclosure. Accordingly, such computer programs may represent controllers of the computer system 150. Where the present disclosure is implemented using software, the software may be stored in a computer program product and loaded into the computer system 150 using the removable storage drive 158, interface 159, and hard disk drive 157, or communications interface 162.

The processor device 153 may comprise one or more modules or engines configured to perform the functions of the computer system 150. Each of the modules or engines may be implemented using hardware and, in some instances, may also utilize software, such as corresponding to program code and/or programs stored in the main memory 147 or secondary memory 156. In such instances, program code may be compiled by the processor device 153 (e.g., by a compiling module or engine) prior to execution by the hardware of the computer system 150. For example, the program code may be source code written in a programming language that is translated into a lower level language, such as assembly language or machine code, for execution by the processor device 153 and/or any additional hardware components of the computer system 150. The process of compiling may include the use of lexical analysis, preprocessing, parsing, semantic analysis, syntax-directed translation, code generation, code optimization, and any other techniques that may be suitable for translation of program code into a lower level language suitable for controlling the computer system 150 to perform the functions disclosed herein. It will be apparent to persons having skill in the relevant art that such processes result in the computer system 150 being a specially configured computer system 150 uniquely programmed to perform the functions discussed herein.

FIG. 21 illustrates a high-level view of an embodiment of the system and some of the services it provides. The system 130 enables an expanded solution set 105 of enabled expanded services that the system 130 may provide. A POS device 101, which may be part of an IoT network, is part of a system framework 180 that interacts with retailers 119, brands 107 and/or other third-party partners. Consumers 200 make purchases at POS 101. Consumer data 062 is collected by system framework 180 across all purchases. Consumer data 062 can be used to provide services 105. Marketing services 501 includes, for example, consumer insights, churn analysis, and media targeting, can be provided to retailers 119 and brands 107. Businesses services 502 includes, for example, inventory monitoring, digital credit, and AI integration. Shopping services 503 includes, for example, brand wallet service, checkout APIs, coupon and rebate management. Food safety and traceability includes, for example, recall management to the consumer, tracking, and notifying retailers of recalled products 504.

FIG. 22 illustrates an embodiment of a system 130. It will be apparent to persons having skill in the relevant art that the embodiment of the system 130 illustrated in FIG. 22 is provided as illustration only and may not be exhaustive to all possible configurations of the system 130 suitable for performing the functions as discussed herein.

The system 130 may also include a digital representation of the POS 152. The digital representation of the POS 152 may be configured to track or facilitate tracking of a POS device in the system. The digital representation of the POS 152 is a business object created and assigned to a POS device (physical or virtual) that interact with and/or within the system 130. The digital representation of the POS 152 allows the system to model and manage the POS device's activity (e.g., authentication, authorization, data and processes, etc.). The digital representation of the POS 152 may also be known as a digital twin of the POS or a digital replica of the POS. The system 130 may assign a digital representation of the POS 152 to every POS device 101 that the system interacts with. By assigning a digital representation of a POS 152 to a POS device the system is able to track the activity of a POS device 101 and give added functionality to the POS device 101. The POS device 101 may be limited in its functionality; however, by assigning a digital representation of the POS 152 to a POS device 101, the POS device 101 may be converted into IoT device adding other enhancements and functionality. Through a digital representation of the POS 152 the online status of a POS device can be tracked through a digital heartbeat back to the cloud. This allows operations staff and/or the system 130 to understand whether POS devices are offline or unused and respond appropriately.

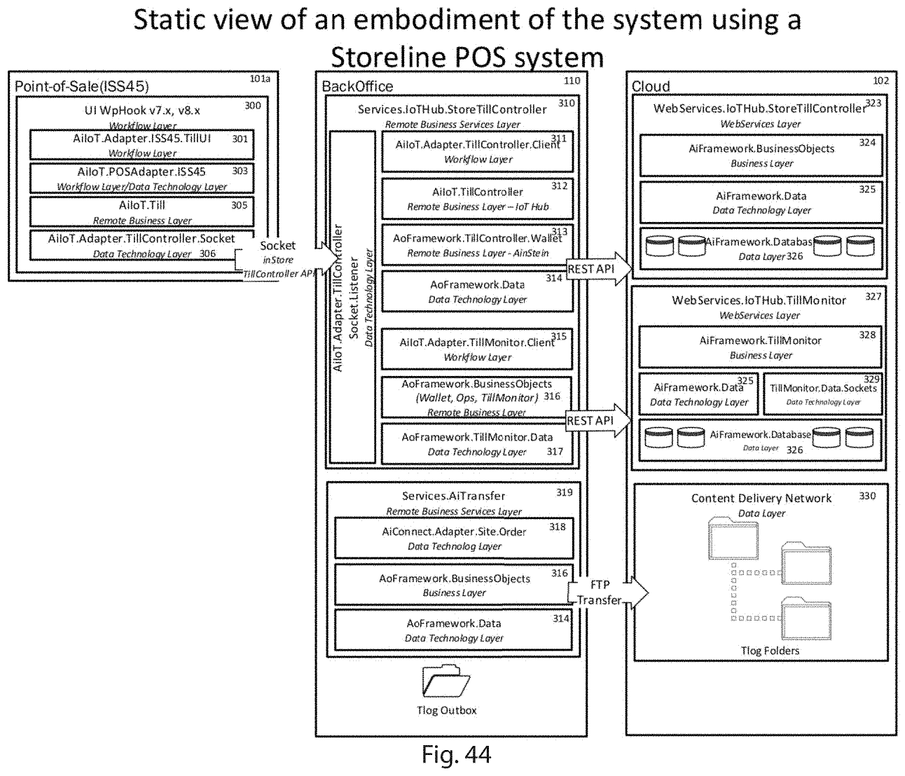

Through a digital representation of the POS 152 pos device events can be submitted to the cloud in real time through the till monitor functionality each event the system values (e.g., cashier login, begin transaction, scan item, cancel item, void item, void order, resume order, subtotal, tender, end transaction, etc.) and post them in real time to the WebServices.IoTHub.TillMonitor 327 (FIG. 44) in the system cloud. When the system cloud services receive these events, they may be relayed to a monitoring dashboard where a store and its POS devices can be displayed, and all active events can be viewed. A digital representation of the POS 152 allows POS device activity to occur both in real time within the system and to have the data archived in a repository for historical record keeping and data verification. The digital representation of the POS 152 allows system communication to the POS device this can be used to stop purchases of products that are currently on recall. The digital representation of the POS technology would recognize the product scan in real time and reject with notification to the cashier that the product cannot be purchased due to a recall (e.g., FDA recall, brand recall, etc.). The digital representation of the POS 152 may be complex or as simple as a numeric value (e.g., GUID). In most embodiments of the system 130, the digital representation of the POS 152 is a key feature of the system 130 and instrumental to receiving data from the POS device.

The system 130 may include a querying module 141. The querying module 141 may be configured to execute queries on repositories to identify information. The querying module 141 may receive one or more data values or query strings and may execute a query string based thereon on an indicated repository, such as the memory 147, servers, etc. to identify information stored therein. The querying module 141 may then output the identified information to an appropriate engine or module of the system 130 as necessary. The querying module 141 may, for example, execute a query on the memory 147 to identify transaction values included in blocks received from the blockchain network/repository 017c.

The system 130 may also include a communication module 148. The communication module 148 may be configured to transmit data between modules, engines, databases, memories, and other components of the system 130 including the POS device 101 for use in performing the functions discussed in this disclosure. The communication module 148 may be comprised of one or more communication types and utilize various communication methods for communications within a computing device. For example, the communication module 148 may be comprised of a bus, contact pin connectors, wires, etc. In some embodiments, the communication module 148 may also be configured to communicate between both internal components of the system 130 and external components of the system 130, such as externally connected POS devices, interne, databases, display devices, input devices, servers, third parties, information feeds, etc.

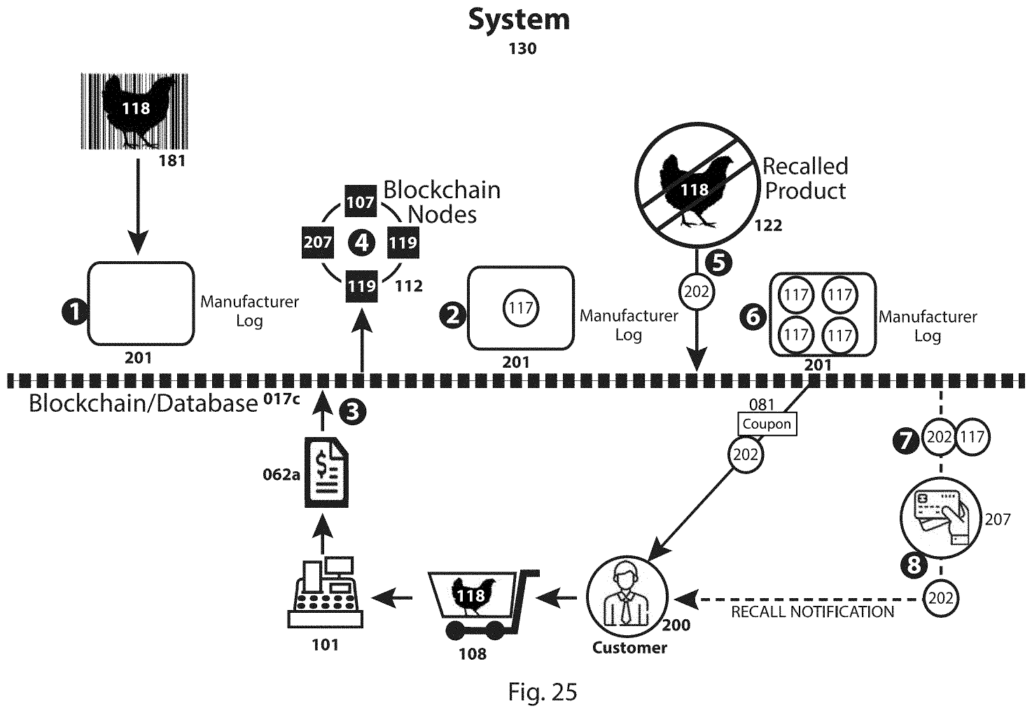

A receiving device may be included in the communication module 148. The receiving device 140 may be comprised of multiple devices, such as different receiving devices for receiving data over different networks, such as a first receiving device for receiving data over a local area network and a second receiving device for receiving data via the Internet. The receiving device may be configured to receive data from payment instruments 209 (FIG. 40), blockchain networks/repositories 017c, the system 130, third-party partners 197, payment processors 207 (FIG. 25), and other systems and entities via one or more communication methods, such as NFC, physical contact points, WiFi, Bluetooth, local area networks, cellular communication networks, the Internet, etc. The receiving device may be configured to receive data over one or more networks via one or more network protocols. The receiving device may receive electronically transmitted data signals, where data may be superimposed or otherwise encoded on the data signal and decoded, parsed, read, or otherwise obtained via receipt of the data signal by the receiving device. In some instances, the receiving device may include a parsing module for parsing the received data signal to obtain the data superimposed thereon. For example, the receiving device may include a parser program configured to receive and transform the received data signal into usable input for the functions performed by the processing device to carry out the methods and systems described herein.

The receiving device may also be configured to receive data signals electronically transmitted by payment processors 207, which may be superimposed or otherwise encoded with notifications indicating approval or denial of electronic payment transactions. The receiving device may be further configured to receive data signals electronically transmitted by payment instruments 209, which may be superimposed or otherwise encoded with digital signatures, public keys, blockchain addresses, or other data used in the identification and authentication of stored in the blockchain and the posting of new wallet, promotion, incentive, consumer safety data thereto.

The communication module 148 may also include a transmitting device. The transmitting device may be configured to transmit data over one or more networks via one or more network protocols. In some instances, the transmitting device may be configured to transmit data to blockchain networks, payment instruments 209, payment processors 207, third-party partners 197, and other entities via one or more communication methods, such as NFC, physical contact points, Bluetooth, radio frequency, local area networks, cellular communication networks, the Internet, etc. In some embodiments, the transmitting device may be comprised of multiple devices, such as different transmitting devices for transmitting data over different networks, such as a first transmitting device for transmitting data over a local area network and a second transmitting device for transmitting data via the Internet. The transmitting device may electronically transmit data signals that have data superimposed that may be parsed by a receiving computing device. In some instances, the transmitting device may include one or more modules for superimposing, encoding, or otherwise formatting data into data signals suitable for transmission.

The transmitting device may be configured to electronically transmit data signals to blockchain networks that are superimposed or otherwise encoded with transaction values, which may include wallet, promotion, incentive and/or consumer safety identification values and wallet, promotion, incentive and/or consumer safety data. The transmitting device may also electronically transmit data signals to blockchain networks/that are superimposed or otherwise encoded with data requests, such as to request new blocks or transaction values for review or use thereof by the POS device 101. The transmitting device may also be configured to electronically transmit data signals to payment instruments 209, such as may be superimposed or otherwise encoded with private keys, public keys, digital signatures, blockchain addresses, or other data necessary for use by the payment instrument 209 to authentication access thereof to a wallet, promotion, incentive and/or consumer safety data. The transmitting device may be further configured to electronically transmit data signals to payment processors 207 and/or a payment network such as may be superimposed or otherwise encoded with transaction data for the processing of an electronic payment transaction, such as payment credentials, a transaction amount, geographic location, store number, POS number, transaction number, time and/or date, currency type, product data, merchant data, credit data, debit data, acquirer data, issuer data, incentive data, reward data, loyalty data, offer data, digital wallet data, promotion data, incentive and/or consumer safety data, product tracking data, transaction logs, etc.

Processor device 153 may be a special purpose or a general-purpose processor device specifically configured to perform the functions discussed herein. The communications module 148 may include a bus, message queue, network, multi-core message-passing scheme, etc. The network may be any network suitable for performing the functions as disclosed herein and may include a local area network (LAN), a wide area network (WAN), a wireless network (e.g., WiFi), a mobile communication network, a satellite network, the Internet, fiber optic, coaxial cable, infrared, radio frequency (RF), or any combination thereof. Other suitable network and/or communication module types and configurations will be apparent to persons having skill in the relevant art.

The system 130 may also include a POS device 101. FIG. 19 illustrates an embodiment of a POS device that could be used with the system 130. FIG. 20 illustrates and embodiment of a computer system that may be used as a POS device in the system.

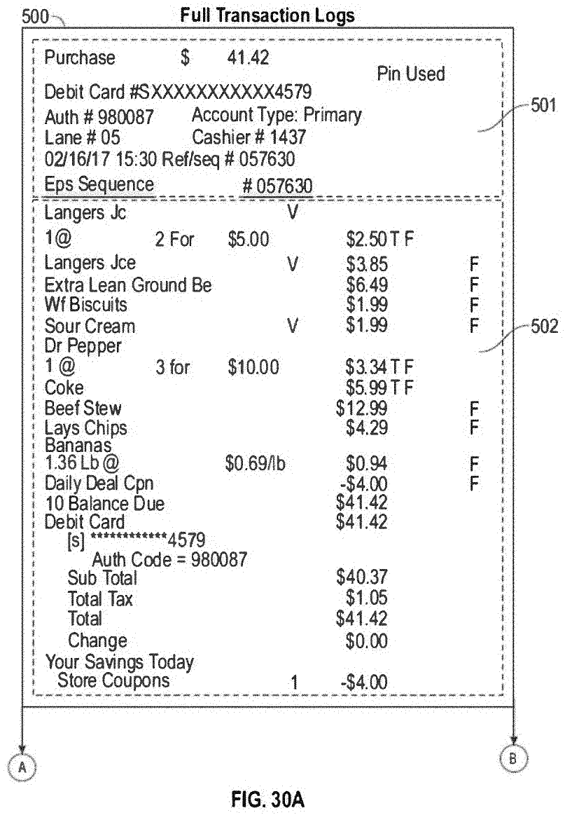

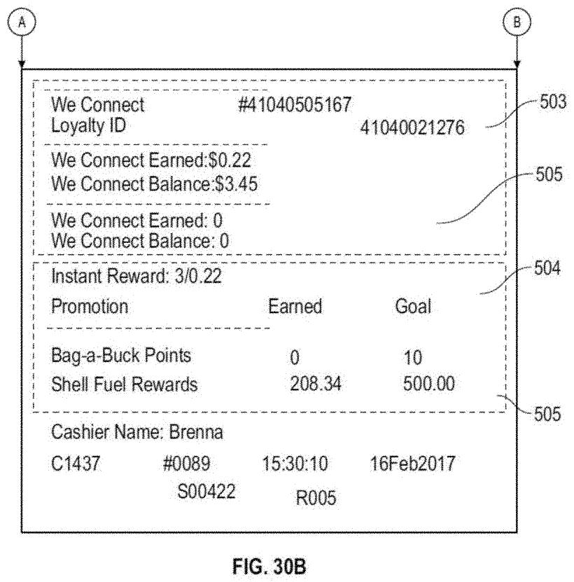

The system 130 may include a back office server 110, in some embodiments the back office server is found in the retailer or merchant 119 (FIG. 21) in other embodiments the back office server is placed in the cloud by putting the till controller 103, AI component 125, etc. on the resource server 026 (FIG. 7) in the cloud 102. As the POS Device 101 completes the transaction, the transaction writes order information to the back-office server 110. The back-office server 110 contains at least two separate binaries, The store till controller service 103 and the TLog transfer service 099. Both of these services reside side by side on the back-office server 110. Both the store till controller service 103 and the TLog transfer service 099 must authenticate 023 with the resource authorization service 027 to receive an access token 024 that is used to allow access to the POS IoT Hub 046/026 (Resource Server). The store till controller 103 is made available to each POS device/cashier checkout 101 via the in store till controller API. Each POS type 101 (e.g., IBM, Retalix, RORC, ScanMaster, etc.) has a POS adapter 060kthat is loaded by the POS device 101 to handle messages specific to the type of POS 101. The POS adapter 060kaccepts messages formed by the type of POS 101 and converts them to the standardized system format and send this information to the till controller 103 using the in store till controller API for processing. At completion of the order the till controller 103 settles order with the POS IoT Hub 046/026 (the resource server 106. At validation of transaction completion the POS device 101 communicates 098 with the till controller 103 for contents to be written to the receipt which could include: food safety details, third-party rewards, point balance, In store credit balance, continuity reward balances, incentive information, or any other information that would be useful to the customer (see FIGS. 30A and 30B). The back-office server 110 stores the order data and batches communication to the CDN 111 using the TLog transfer service 099 or similar. The till controller 103 and the AI component 125 can to be placed in the cloud 102 to service as the back-office server 110. Other suitable back office server types and configurations will be apparent to persons having skill in the relevant art.

The system 130 may also include informational feeds 196. Information feeds 196 come into the system via the communication module 148 and can contain any information the system finds valuable. The informational feeds may contain information from a government agency (e.g., Centers for Disease Control and Prevention (CDC), FDA) and may contain information of product recalls. It could contain weather information, for example. Other suitable informational feeds will be apparent to persons having skill in the relevant art.

The system 130 may also include location services 129. Location services 129 help locate customers, products (during shipping), retail locations, etc. Location services 129 may use GPS employ the use of a customer's device, proximity beacons, cameras, facial recognition, triangulation, other sensors and tools to determine a customer's location in order to send timely, information, notices and/or personalized incentives to a customer. The location may be generalized (e.g., at retail store location) or more specific (e.g., halfway down aisle #4 of retail store #2). Location services 129 can also help with the gamification of incentives where a customer performs certain tasks (some of which may be location dependent) in order to receive a reward. Location services may help with the tracking of products to provide estimates on just in time inventory for retail locations and/or manufactures. Location services 129 could also help with the management of livestock and animals. Combined with AI components 125, location service can assist in providing data in order to estimate, predict, recommend and/or make offers for third-party partners, manufacturers, farms and/or customers. Other suitable location services types and configurations will be apparent to persons having skill in the relevant art.

The system 130 may also include a processor module. The processor module 153 could be made of a single device or multiple devices and engines. The processor module 153 might include a data processor and data processing engines. The processor module 153 may include central processing units, graphical processing units, vision processing units,

Tensor processing units, neural processing units, physics processing units, digital signal processors, image signal processors, microprocessors, multi-core processors, super scalar processors, etc. The processor module 153 may be linked to the communications module. Other suitable processor types and configurations will be apparent to persons having skill in the relevant art.

The system 130 may also include interfaces and input devices. Interfaces and/or input devices are used to interact with the system 130. These devices might include keyboards, displays, cameras, haptics, buttons, brain communication interfaces, scanners, infrared signals interfaces, radio signals interfaces, WiFi interfaces, Bluetooth interfaces, NFC interfaces, compasses, gyroscopes, accelerometers, GPS, touchscreens, GUI, phones, computers, thermometers, thermal imaging, microphones, sensors, biometric sensing devices, facial recognition interfaces, object recognition interfaces, optical florescence sensors, ultrasonic sensors, web searches tools, location beacons, data feeds interfaces, sensors of all types. These input and interfaces could be used to input or export data from the system 130. Other suitable input and interface types and configurations will be apparent to persons having skill in the relevant art.

The system 130 may include webservices 014a. They provide similar advantages as business object components 015, such as interoperability and loose coupling to enable high reuse and development efficiency. A powerful difference is webservices are provided over a network and are technology independent. This allows webservices to be shared across businesses and provides a new level of interoperability. Webservices may be implemented using SOAP or REST technologies. An adapter encapsulates this technology and maps the webservice interface to the system 130. Webservices use the OAuth 2.0 standard or other standard in which client credentials obtain an access token from the resource authorization server 027 to pass security 195. Once the access token is received, it is authorized for transactions once the remaining client space information successfully authenticates.

The system 130 may include many servers or virtual machines 106. These servers or virtual machines are organized as authorization servers 027 and resource servers 026. Authorization servers handle the security 195 for the system. Authorization servers 027 issue access tokens that give internal and external systems access to resource servers 026. Access tokens are requested from an authorization API that authenticates the client using OAuth 2.0 standard or other standard and upon validation the access token is returned and can be used to access resources available on resource servers 026. Other suitable authorization types and configurations will be apparent to persons having skill in the relevant art.

The system 130 may organize resource servers 106 based on internal and external services. Business services 014b are internal services responsible for critical business support functions. These business services 014b can be communicated to through a RESTful API for internal control. Business services 014b are built to scale by using multiple instances of the same service assigned to a different space definition. Business services 014b include several services such as POS-IoT order harvesting services, store and national product harvesting services, coupon management services, notification services, reward services, caching services, job scheduling services to name a few. Other suitable business service types and configurations will be apparent to persons having skill in the relevant art.

The system 130 may capture all rules and data that support all application services 014 in business objects 015. Therefore, the common set of data and rules are captured in a single business layer that is loosely coupled to other layers of the system 130. This allows application services 014 to utilize the same rules and data rather than duplicating this effort. Business objects 015 represent the collective intelligence of a system and therefore there are many business objects within the system 130. The business objects 015 are organized into modules or assemblies that contain highly cohesive objects. They include modules such as wallet, order (e.g., TLog), product, operations, security, promotions, payments, communications to name a few. Other suitable business object types and configurations will be apparent to persons having skill in the relevant art.

The system 130 may include numerous sources of data. These data sources are communicated to using data technology components. Data technology components 016 are represented by a common object-oriented adapter pattern. These adapters are responsible for allowing two entities with different interfaces to communicate. These data technology components 016 are organized into modules or assemblies that contain highly cohesive adapters. These include modules such as database adapters, blockchain adapters, payment processor adapters, TLog order adapters, TLog order provider adapters, coupon adapters, coupon provider adapters, till controller adapters, scale controller adapters, POS adapters, till monitor adapters, promotion clearinghouse adapters, product file adapters, reward adapters, product subscriber adapters, site order adapters, and site product adapters. Other suitable data technology component types and configurations will be apparent to persons having skill in the relevant art.

The system 130 may incorporate numerous sources of data and/or repositories 017. This includes relational database sources, blockchain sources, files, third-party WebAPI sources, IoT devices, socket sources, and other legacy device sources. Data technology component 016 is used to communicate to these data sources 017 through adapters. The adapters have a standard interface so that complex business service logic can be written to manage high volume communication with these data sources 017. Adapters for data technology component 016 can be easily plugged into these business services so that integration with data sources 017 is scalable and reliable. Other suitable data source types and configurations will be apparent to persons having skill in the relevant art.

The system 130 may work with various third-party partners 197 both importing and exporting data to and from these partners. These partners 197 are considered data sources 017. Integration to these partners 197 occurs through data technology component adapters 017. The system's 130 business object components 015 utilize these data technology component adapters 017 to encapsulate third-party integrations away from application services. This shields application services 014 and presentation applications from rapid changes with third parties 197. Other suitable third-party partner types and configurations will be apparent to persons having skill in the relevant art.

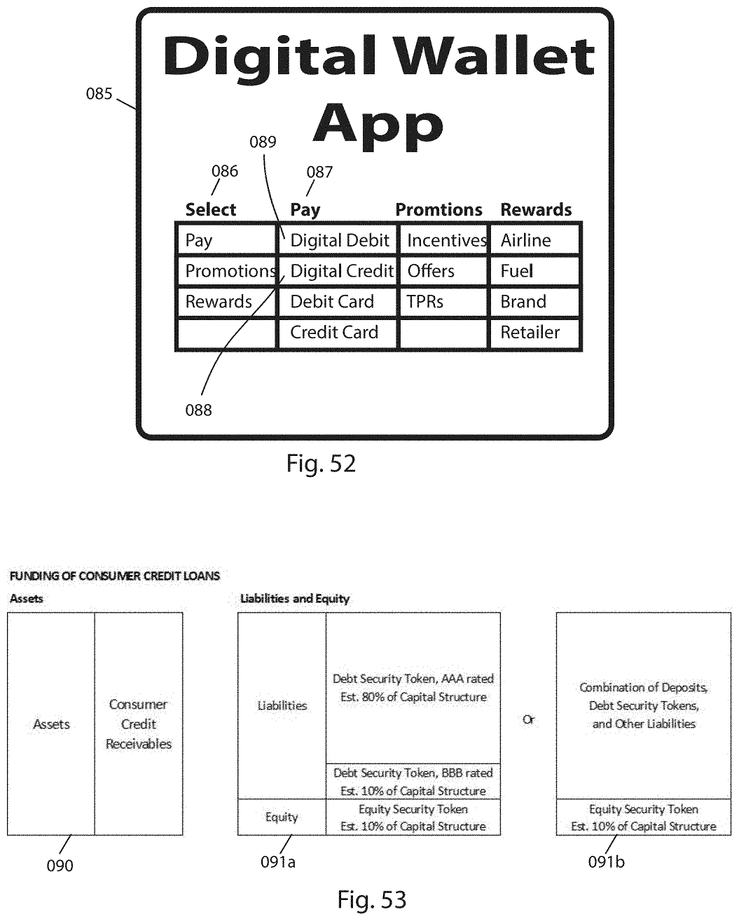

The system 130 may include AI components 125. AI components 125 may develop personalized pricing for consumers and deliver tailor-made discounts based on location, date, time, age of customer, sex of customer, passed behavior, and other criteria. AI components 125 may offer an incentive a customer for a second location based on past purchases and location of a customer. This not only ensures consumers will see what is most relevant to them, but also ensures that brands are not incentivizing consumers unnecessarily. Games (i.e., gamifcation of incentives or games to promote good will) can be suggested or personalized for consumers 200 or groups of consumers as well. A group of consumers may all opt in to compete against each other for a prize (incentive, reward). AI may create a game based on criteria of every member of the group or other criteria. The group could be given tasks to complete. Those tasks could be sent to individual communication devices 085 (FIG. 52) or printed to a customer receipt 094.

AI components 125 could suggest responses, predict outcomes (e.g., damaged products, etc.), diagnose, anticipate (e.g., ETAs, just-in-time deliveries, etc.), incentivize (e.g., personized incentives based on multiple criteria), recommend, create games, and more.

Through real-time monitoring and logging of POS-IoT activity and WebAPI usage, AI components 125 can be used to help with:
Real-time awareness of operational issues and predict required help with common misuse of POS or WebAPIs before they turn to bigger issues;
Predicting service and repository sharding needs based on POS and WebAPI activity; and
Making available real-time reports that help with more agile response to issues.

AI components 125 may also help with real-time product scanner throughput to help with brand and retailer scan-based incentives, real-time reward threshold progress, recognition of customer at checkout, real-time access to store, mobile and cloud orders and real-time product inventory awareness minute-by-minute.

The system 130 may include a solution set (and/or services) 105. The solution set and/or services are services provided by the system 130 to customers 200, retailers 119, brands 107, third parties 197, etc. These solutions and services may cover areas such as food and consumer safety, traceability, marketing, shopping, business, and operations. More specifically some of the solutions and services may include:

Consumer Recall Management—Recall notices and information managed and communicated via email, SMS, in-app and mobile notifications, mobile geofenced notifications, social media, printing on receipts, and other available channels.

Retailer Recall Management—alert stores, managers, and companies with recall information.

Traceability—Systems that enable and streamline the tracing of recalled products within the distributor, wholesaler, and retailer businesses.

POS Recalled Item Stop—The ability to stop the sale of any recalled product to consumers at the POS.

Food Alerts—The ability to alert a consumer that they may have purchased a product with ingredients that could be harmful to them or their family based on analysis of purchase history or by direct input into a system by the consumer.

Inventory Monitoring—Automated alerts and triggers for products sold in stores.

POS and Backoffice Product Item File Management—Automated management and viewing of products loaded into back office or POS systems.

Artificial Intelligence—AI as a service, to organize, predict, prompt, diagnosis, recommend, create, etc. by analyzing and using data.

Quick Enrollment—The ability to enroll consumers (for a service or wallet) in lane within a transaction.

Intelligent Offer Management Across Systems—A service that provides a wholesaler or retailer the ability to enable and disseminate simple or complex offers into disparate back office or POS systems from one interface.

Scan-based Incentive Management—A service that securely automates the processes of accumulating product sales data on items sold and relaying such securely to brands for the payment of incentives or other to retailers.

Checkout APIs—A service that provides a method for one or many vendors to connect to either brand or retailer systems for the purposes of creating additional checkout methods (mobile scan and checkout, online shopping, POS-less POS, smart store, etc.).

Digital Wallet—A service that includes open loop and closed loop debits, credits, rewards, crypto, digital credit, third-party payments, etc. at the POS, mobile or online.

Digital Credit—A novel way of offering credit to consumers that may originate at the POS.

Coupons and Rebate Management—A service that allows the full management of digital coupons and rebates. Enables consumer targeting, continuity programs over multiple purchases at one or many locations.

Brand Wallet Service—A service that allows brands to disseminate offers directly to consumers for use at an array of retailers within desperate verticals or markets. A service that allows a brand to reward one or more consumers based on certain data or criteria. A service that allows consumers to automatically have their purchases written to certain brand databases or blockchains for the purposes of being rewarded or incented. A service that allows brands to securely build marketing programs that include an array of third-party participants and incentives.

Analysis Engine—Services that provide operational dashboards that are connected to back office or POS systems, such as insight and shopper churn.

Shopper Targeting Service—A service that enables consumer targeting via the results of the analysis engine or other third-party data. The targeting service in conjunction with the AI service enable personalized discounts and pricing of products for individual consumers.

Mobile Proximity—A service that creates and manages multiple mobile applications along with connections to the systems for the purposes of delivering and transacting geo fenced or proximity offers, coupons, rebates, and other.

Rewards Engine—A service by which to manage reward programs connected to retail such as fuel rewards, points, airline miles, third-party media rewards (Netflix, Microsoft® Xbox, etc.).

Charity Management—A service by which to fully manage 1,000's of charities tied to brand or retail purchases.

Deal Engine—A service that manages time sensitive or quantity-based retailer of brand generated deals that also connects to retailer inventory making sure enough product exists to fulfill consumer digital selection/opt-in.

(POS and BackOffice) IoT and IoT Hub Monitoring—A service may monitor device authentication and authorization, device communication telemetry, device-to-cloud messages (API and websocket), order and product file uploads, real-time activity of store and cloud till controller IoT Hub events and data, cloud-to-store IoT Hub communication, etc.

OnBoarding and Configuration Services—Provides services to board brands and retailers; define, load and configure brands and retailers space and service configuration; define, load and configure POS and BackOffice IoT and IoT Hub spaces; provide provisioning services at IoT startup; etc.

Other suitable solutions and services types and configurations will be apparent to those skilled in the relevant art.

The system 130 may include business object component services 015. A series of assemblies that encapsulate data and process rules that support the system solutions 105. These assemblies are used by all web applications, business services, and webservices. They are the business objects for development within the system. The assemblies encapsulate high risk and complex algorithms, such as all database CRUD and sharding operations and common rulesets used within the system. These assemblies ensure high reuse, limit common coding mistakes, ensures consistency across applications, eliminates competing rulesets, and keep application developers focused on the needs of the application, UI or service tier (instead of rewriting all code). These assemblies are in portion exposed through webservices 014a to allow third-party partners 197 to access critical business functions using their own preferred service and web application tools.

Notable functionality includes ORM 060a and OBM 060b that establish a unique consistency of encapsulated transaction management, execution of critical rules, unique sharding capability based on physical space definition and relational database CRUD interactions and/or blockchain that require little to no custom coding.

The system 130 may also include business services 014b. Business services 014b are internal services responsible for critical business support functions. These business services 014b can be communicated to through a RESTful API for internal control. Business services 014b are built to scale by using multiple instances of the same service assigned to a different space definition. Business services 014b include several services such as POS-IoT order harvesting services, store and national product harvesting services, coupon management services, notification services, reward services, caching services, job scheduling services to name a few.

Notable functionality includes: allowance of multiple instances of a business service to be created to help distribute scale to enterprise throughput; threading algorithm that allows multiple worker threads to efficiently and safely work independently to scale the processing of required work; ability to take POS 101 native TLog formats and process these file formats into a normalized format; ability to import different third-party coupon formats and process these into a normalized promotion format; ability to work with third-party coupon clearinghouses to settle redeemed coupons for retailers; ability to import national product information from different third-party product providers; ability to import store product information including weighted items from each store in the network; ability to process scan-based incentives to determine benefit to retailers; ability to process in bulk third-party rewards obtain through meeting promotion thresholds that give benefits outside of more normal store product rewards; and ability to cache critical business object component data to help frequent read-only operations scale.

The system 130 may include webservices 014a. They provide similar advantages as business object components 015, such as interoperability and loose coupling to enable high reuse and development efficiency. A powerful difference is webservices are provided over a network and are technology independent. This allows webservices to be shared across businesses and provides a new level of interoperability.

Notable functionality includes the ability to easily expose any system functionality with little to no coding by building a RESTful wrapper around business object components 015. Includes webservices for system authentication, wallet, operations, promotion, product, order, and cloud-based till controllers 331, 337 functionalities.

The system 130 may also include remote services 029, which include a remote presentation tier 031 and a remote business tier 032. Remote services may monitor product processing at remote retail store locations. When a remote store scale controller 343 is present a scale monitor adapter 347 can be utilized to send weighted item product scale information real-time to the system cloud data center 102. This information can be used to monitor real-time activity on store product scales.

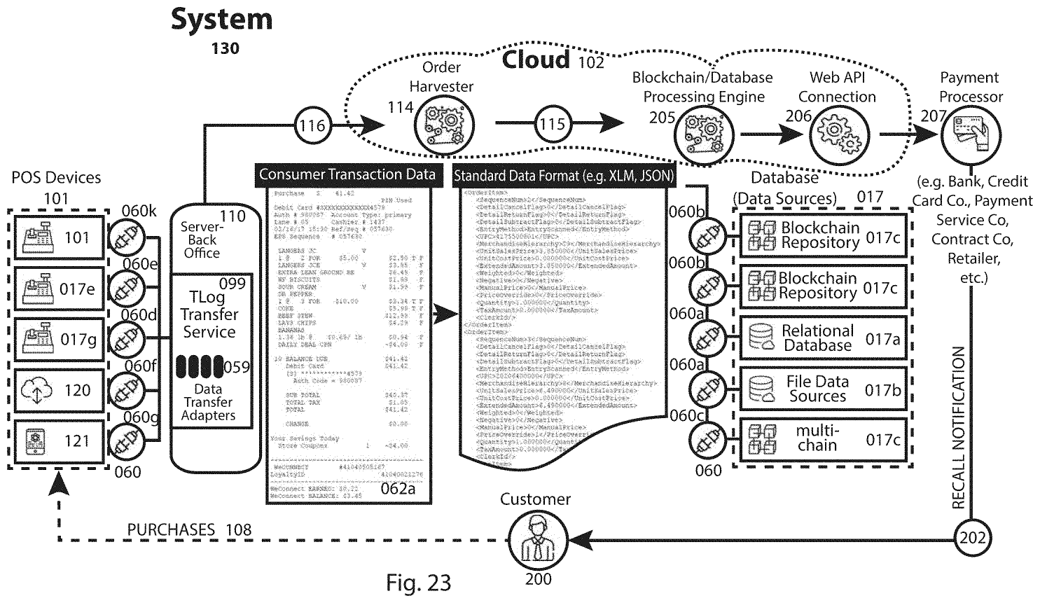

FIG. 23 depicts an embodiment of the system showing data flow in the event of a product recall. The customer 200 makes a purchase 108. The purchase transaction is finalized at the POS 101 (e.g., legacy POS 017g, IoT POS 017e, cloud-based POS system 120, mobile checkout POS system 121, or other type of POS). Data processing adapters 060 (e.g., POS adapter 060k, socket data source adapters 060f, IoT device adapters 060e, webservice data source adapter, legacy POS system adapter 060d, or other connecter/adapter) are used for real-time interaction with the customer 200 during checkout. The POSs 101 are connected to the back-office server 110, which contains the TLog transfer service 099. The TLog transfer services 099 loads the appropriate data transfer adapter 059. The data transfer adapter 059 knows where the back-office POS stores transaction logs for the type of POS 101 being used and writes transaction logs to that location. The data transfer adapters 059 transfer these logs in native POS language format 116 to the c102 to the CDN and on to the order harvester 114, which determines the type of file based on POS 101 types and loads the appropriate order adapter to process the file. Order adapters automate complex data normalization so multiple sources of data are converted into a standard system language format 115.

The blockchain/database processing engine 205 enables adapter technology to securely write trusted data to any blockchain network/repository 017c or database 017b. The data 062a is then stored in repository(s) 017 (e.g., blockchain repository 017c, Relational database 017a, file data source repository 017b, or other database). Ideally, all the data is stored in a database 017a and a select portion of the data 062a is also stored on a blockchain repository 017c, but as long as the data 062a is secure and accessible, any manner of storage is acceptable. The connections to the repository/databases are made via data processing adapter 060 (e.g., ORM adapter 060a, OBM adapter 060b, file adapters 060c, or other connector/adapters). In the event of a recall issued by a brand or government agency, the payment processor 207 is notified via a Web API connection 206. The Web API connection allows clients and partner blockchain and/or database networks to access clean, trusted data through standard communication protocols. Via the Web API connection 206, the payment processor 207 learns which of their customers 200 have been affected by the recall and the payment processor 207 or their agent manages the recall notification 202 to the customer(s) 200.

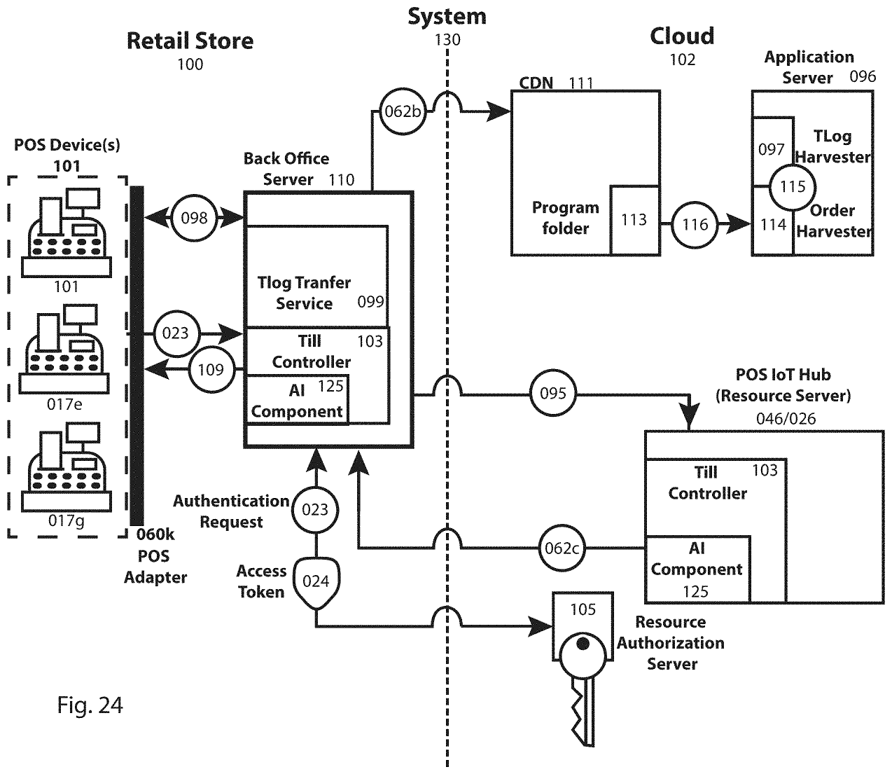

FIG. 24 depicts an embodiment of the system showing data processing flows. The order communication process manages transaction orders from POS devices 101, 017g, 017e within retail stores 100 (both online and brick-and-mortar) to communicate with cloud 102 storage. When a consumer attempts to close a transaction, the POS device 101 communicates with the till controller 103. This authentication communication 023 is requesting OAuth 2.0 authorization (or other secure authorization standard) from the resource authorization server 027 in order to access the POS IoT Hub (Resource Server) 046/026. The IoT Hub 046/026 converts any legacy POS device 017g into IoT POS device 017e by making a digital representation of the legacy POS device 017g that is IoT compatible. Once this authorization is confirmed an access token 024 is issued and the till controller 103 requests any relevant data 095 for the transaction 062c. This historic transaction data 062c includes all name, wallet information, promotions available, points, and other transaction history relevant to the purchase 109. The POS device 101 prior to order completion requests order adjustments (e.g., benefits in the form of store credit, promotion discounts, food safety information, etc.) from the till controller. The POS device 101 upon completing the transaction writes order information to the back-office server 110. The back-office server 110 contains at least two separate binaries, The store till controller service 103 and the TLog transfer service 099. Both of these services reside side by side on the back-office server 110. Both the store till controller service 103 and the TLog transfer service 099 must authenticate 023 with the resource authorization service 027 to receive an access token 024 that is used to allow access to the POS IoT Hub 046/026 (resource server). The store till controller 103 is made available to each POS device/cashier checkout 101 via the in store till controller API. Each POS type 101 (e.g., IBM, Retalix, RORC, ScanMaster, etc.) has a POS adapter 060k that is loaded by the POS device 101 to handle messages specific to the type of POS 101. The adapter 060kaccepts messages formed by the type of POS 101 and converts them to the standardized system format and send this information to the till controller 103 using the in store till controller API for processing. At completion of the order the till controller 103 settles order with the POS IoT Hub 046/026. At validation of transaction completion the POS device 101 communicates 098 with the till controller 103 for contents to be written to the receipt which could include: food safety details, third-party rewards, point balance, in-store credit balance, continuity reward balances, incentive information, or any other information that would be useful to the customer.

The back-office server 110 stores the order data and batches communication to the CDN 111 using the TLog transfer service 099 (or similar). This batch communication 062b mitigates server calls to manage the large amount of store transactions. The CDN 111 organizes order files by program folders 113 to efficiently store this data 062. The data stored on the CDN 111 is in native POS language format 116. This order data is then harvested by the order harvester service 114. In harvesting the data, the native POS language format 116 is converted into a standardized (normalized) data format 115 that is universal to the system.

The AI component 125 of the till controller 103 takes the scan activity at the POS 101 and determines the benefits to be added on the customer's behalf to the order. It should be noted that in order to support online ordering and mobile checkout the till controller 103 is also within the POS IoT Hub 046/026. The AI component 125 can also be located in the cloud to allow us to support the migration to cloud 102 and mobile based POS systems 121. The till controller 103 and the AI component 125 can to be placed in the cloud to service as the back-office server 110.

It should be noted that the system 130 is able to provide real time (or near real time) views of data 062 (e.g., as products pass through a POS system 101) and also store data in repositories 017a, 017c, etc.

FIG. 25 depicts an embodiment of the system showing alerts to a customer for a recall of a purchased item. A unique manufacturer log 201 is created for each UPC 181 tracked within the food safety tracking system. These manufacturer logs 201 are clustered within a larger manufacturer log and hold a consumer GUID (or customer identifier) 117 and transaction data 062a for each customer 200 that purchased a product 108 that carries this UPC 181.

Consumer information 062a is anonymized and only connected to consumer's PII in the event of a recall by the payment processor 207 (e.g., bank, credit card company, payment services company, or other contract company). The food safety tracking system 130 may include the following steps (not necessarily in this order):

Creation—brand-specific manufacturer logs 201 are created within the food safety tracking system 130 based on a UPC 181 that is assigned to a product 118 by a brand 107 (a unique manufacturer log 201 for each unique UPC 181). A manufacturer log 201 houses consumer GUIDs 117 for each consumer 200 that has purchased the product carrying the UPC 181 that is associated with that manufacturer log 201.

Confirm Purchase—customer 200 purchases 108 a product 118. Customer 200 pays for the product 118 at the POS 101 and transaction information 062a is generated. Validated consumer and product purchase information (transaction data) 062a is written the blockchain 017c or other secure database utilizing information obtained from the POS 101.

Node validation—all partner brands 107, retailers 119, and/or payment processors 207 that participate in node 112 validation confirm the write to the blockchain 017c or other secure database.

Issue recall—a brand and/or a government agency issues a product recall 202.

Manufacturer log identified—recalled UPC 181 is matched with its manufacturer log 201 that contains all consumer GUIDs 117 that represent customers 200 who purchased the recalled product that carries the recalled UPC 181.

Payment processor notification—the appropriate payment processors 207 (those payment processors 207 that processed a payment for the recalled product 118) are notified of the product recall 202 along with the consumer GUIDs 117 that purchased the recalled product 122.

Customer notification—the payment processors 207 matches the consumer GUIDs 117 with the consumer 200 and leverages their current consumer network to notify 202 those consumers impacted by the recalled product 122.

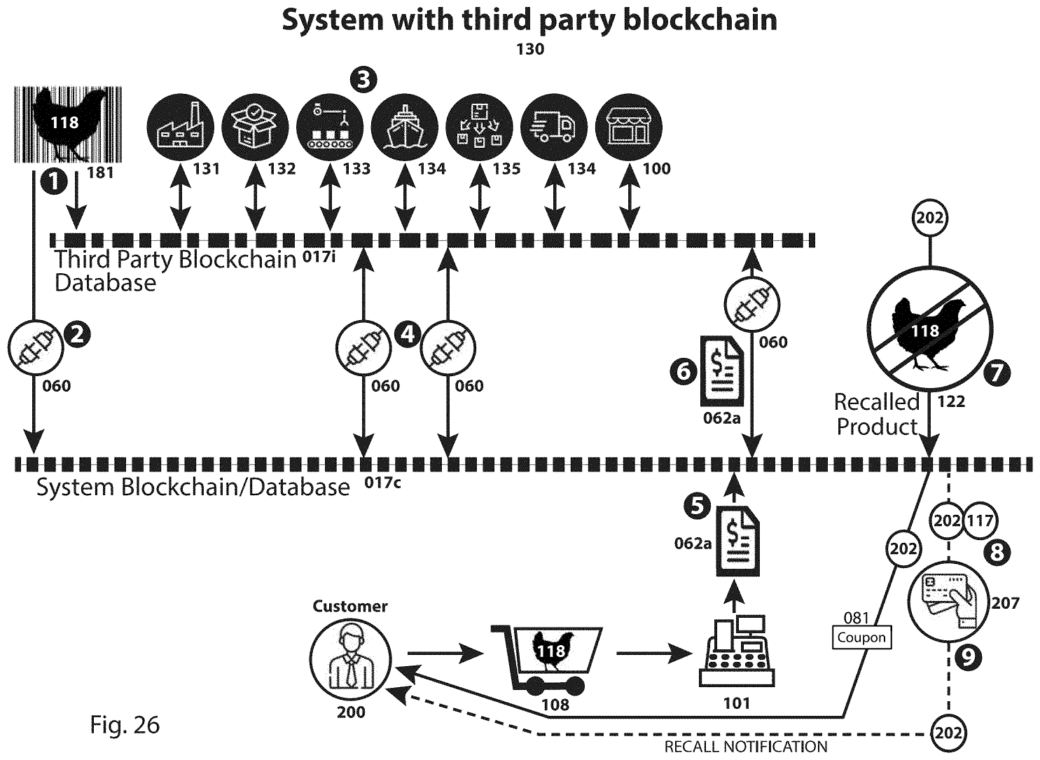

FIG. 26 depicts an embodiment of the system 130 that uses one or more third-party blockchains that are used as outlined in the following steps (but not necessarily in the recited order):

Creation—a brand specifies a UPC 181 for its product and registers the UPC 181 with a third-party blockchain network 017i.

Connect the brand registers the UPC 181 with the system's blockchain and/or database 017c.

Product progression—during each step of the production and delivery process (production 131, packaging 132, shipping 133, transportation 134, distribution 135, other transport 134, delivery to retail 100), the product 118 is tracked via the third-party blockchain 017i.

The system 130 is approved with the third-party blockchain 017i to write on behalf of the brand. The system 130 writes to its own blockchain/database 017c and the third-party blockchain 017i on behalf of the brand. Using data processing adapters (e.g., block chain adapters) 060. The system's blockchain adapter technology 060 is used to write to brand registered third-party blockchain networks 017i.

Confirm purchase—validated consumer 200 (via GUID 117) and product 118 purchase 108 written to system's blockchain network 017c (or database) utilizing POS 101 transaction information 062a.

Purchase information is also written to the third-party blockchain network 017i via the system's block chain adapters 060.

Issue recall—the brand or a government agency issues a product recall alert 202. The system connects to brand API and finds impacted consumers 200 via their GUID 117.

Payment processor notification—the system 130 notifies the appropriate payment processors with consumer GUID 117 and recalled product 122 information.

Consumer notification—payment processor leverages their consumer network to notify 202 those impacted by recall.

In some embodiments, all processes could take place on the system's blockchain 017c or other secure database without the third-party blockchains 017i.

FIG. 27 depicts a possible flow for a product recall using a smart contract within the system 130. A product 118 is recalled, such as when a brand or government agency issues a product recall alert 202. The system 130 has collected and stored information 062a on a blockchain database 017c and/or other secure database 017a from POS 101 scan (receipt information) and payment transaction data 062a. The native POS language 116 is converted to a system standard language 115. When the recall alert 202 is issued, the system 130 identifies customers 200 (using a GUID 117) that have purchased the recalled product 122. The data is correlated. GUIDs 117 represent customers 200 that have purchased the recalled product 122 and are correlated with the payment processor's 207 consumer network and the payment processor manages the alert 202 that is sent out to the impacted customer's devices 123. This correlation may take place via smart contract. A smart contract is written between the system 130 and the payment processor 207. The payment processor 207 governs the notification procedure that takes place when a recall alert notice 202 is issued and the notification of the impacted customers 200 (via a customer device 123) who purchased a recalled product 122. The payment processor 207 has contact information for customers 200 who the payment processor 207 helped facilitate payments to a third-party from whom the customer 200 purchased a now recalled product 122. The system 130 has information regarding the customer GUID or other customer identifier 117 that anonymously identifies the customer 200, the recall alert 202, and the recalled product 122, and the payment processor 207 has information regarding the customer's 200 name and contact information that corresponds to the GUID 117.

A smart contract triggering event occurs when a product recall 202 is issued by a brand or government agency, and the system 130 notifies the payment processor 207 (via smart contract 048 or other method) about the recall alert 202 with corresponding information regarding recalled product 122 and GUID 117 for customers who purchased the recalled product 122. The payment processor 207 then alerts 202 the customer 200 of recalled product via a customer device 123. Alternatively, name and contact information for customer 200 are held (on a blockchain or other database) anonymously (to all entities except the payment processor 207 and its agents) and, upon the trigger event, the smart contract 048 ensures that the customer 200 is automatically sent a product recall alert 202 via a customer device 123.

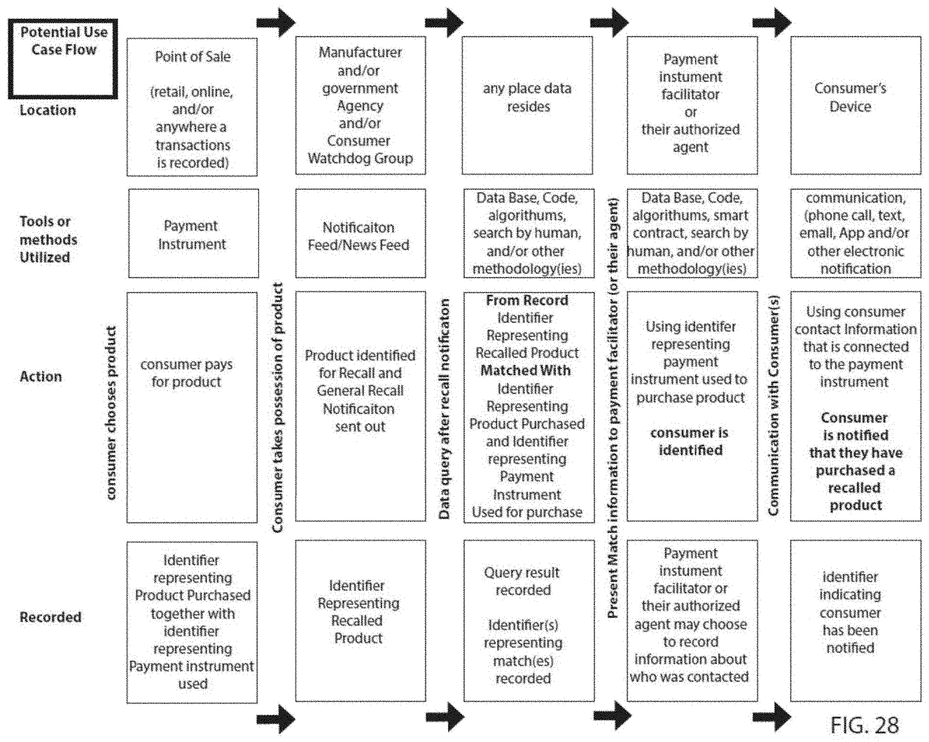

FIG. 28 illustrates a flow of actions from a customer's purchase of a product to receiving a recall notice for the product. Showing location, action, method of preforming the action, and data recorded in an example embodiment.

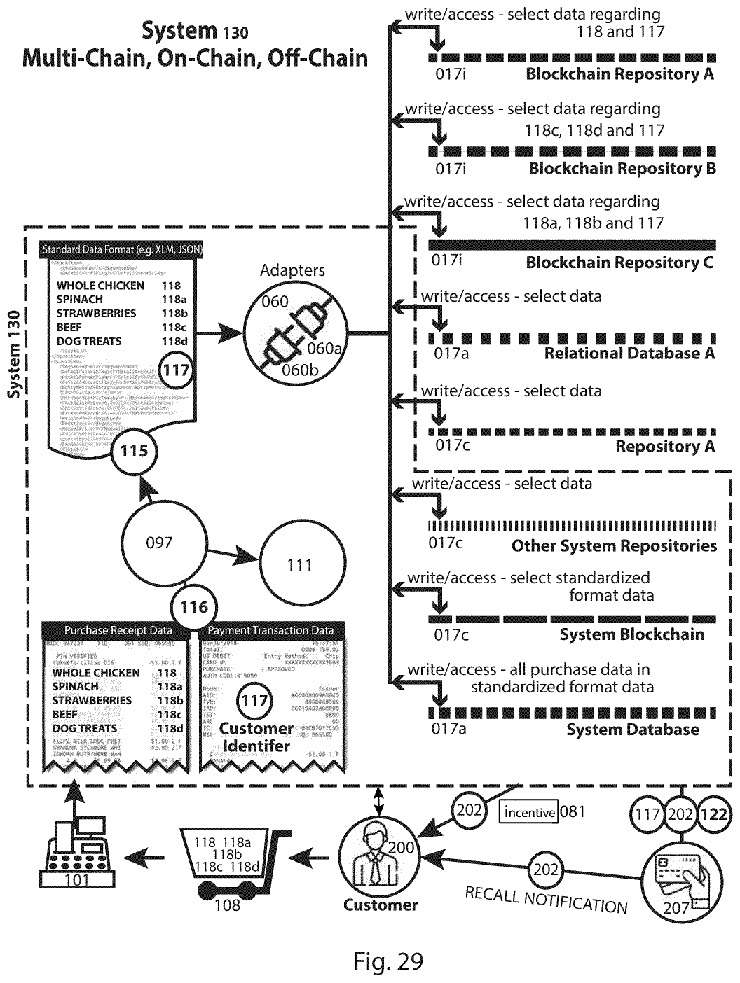

FIG. 29 illustrates an embodiment of system 130 that uses on-chain, off-chain, and multi-chain technologies. When a customer 200 purchases 108 a number of products 119 at a POS 101 the TLog harvester 097 combines the product 118 information and the customer identity 117 and stores it within the CDN 111 and converts the POS language format 116 into a standard format 115. This information 115 is then written to various databases 017i, 017a, 017c through a series of adapters 060. The ORM adapter 060a leverages identity services 020 to map the data to the correct relational database 017c. The OBM adapter 060b leverages the same identity services 020 to write to the appropriate data to various blockchain repositories 017i. In this example, beef 118c and dog treats 118d are stored on blockchain repository B 017i. Spinach 118a and strawberries 118b are stored on blockchain repository C 017i. The OBM adapter 060b understands which product 118 belongs on which blockchain 017i and writes the product and customer identifier 117 appropriately. Then, in the event of a recall, a notification 202 can be sent to the consumer 200. This can occur with an incentive 081 or the consumer information 117, product information 202, and recall information 122 can be shared with the payment processor 207 and they 207 can handle the recall notification 202.

FIGS. 30A and 30B depict a transaction log 500 according to one embodiment. Traditional POS devices make it difficult to access full transaction data. Many cloud services only document basic information such as time, location, and total transaction cost. Embodiments disclosed herein transmit hundreds of transaction dimensions to empower every aspect of a business while providing near real-time data from POS activity. Section 501 comprises basic transaction information. This includes topline purchase information, such as total cost, store data, and payment type, which is accessible to most POS services. Each order associated with a transaction log is given a single OrderID GUID. Each customer is given a CustomerID GUID. As the orders are harvested, the customer is associated with their order. The GUIDs can be stored on the blockchain.

Section 502 comprises the full transaction log, which transmits critical details of transactions from POS systems. IoT services modernize hardware and transform previously isolated devices into writers on the blockchain. Section 502 includes purchase details, including UPCs, product count, product cost, and promotions and discounts. UPCs can be used to unlock a myriad of product dimensions, such as product classification, nutrition information, manufacturer information, and recall information. The information in section 502 can be used to validate inventory, document sales, validate promotions, flag stores for product recall, track product movement, and manage customer notifications in the event of a recall.

Section 503 comprises consumer information. This information allows detailed sales data to be connected to consumer loyalty accounts where available. This unlocks consumer outreach, customer relationship management (CRM), and enables proactive recall management.

Section 504 comprises tiered promotion information. Businesses can incentivize repeat visits through stackable promotions. Any additional information 505 can be written on the transaction log or customer receipt, such as consumer safety entity information, rewards account balances, new incentives, gamification messages (e.g., next clue in a treasure hunt, a task to perform for an incentive, coupons, list of tasks (competed and/or incomplete), website or social media site to visit, etc.), thought of the day, any information of interest or value.

Table 1 is a JSON format for use in transmitting order data in one embodiment.

TABLE 1

| | | | |
|---|---|---|---|
| Object Name | Field Name | Type | Description |

Order

| | | |
|---|---|---|
| StoreNum | Integer | Identify store where the transaction took |
| | | place. |
| BusinessDate | String | [YYYY-MM-DD] Business Date of the |
| | | Transaction. |
| TransactionNum | Integer | Number associated with transaction |
| TillNum | Integer | Identify terminal where the transaction took |
| | | place. |
| StartDateTime | String | [YYYY-MM-DDThh:mm:ss] Date and time |
| | | of transaction start |
| EndDateTime | String | [YYYY-MM-DDThh:mm:ss] End of |
| | | Transaction checkout |
| CashierNum | Integer | Unique identifier for cashier |
| CancelFlag | Integer | Was the transaction cancelled or voided |
| TrainingModeFlag | Integer | Was the transaction run in training mode |
| OfflineFlag | Integer | Was the POS offline for the transaction. |
| SuspendFlag | Integer | Was the transaction suspended to be |
| | | completed at a later time. |
| RefundFlag | Integer | Was the transaction a refund transaction. |
| TaxExemptFlag | Integer | Was the transaction exempt of taxes |
| ManagerOverrideFlag | Integer | Was the manager involved in overriding the |
| | | transaction. |
| TotalAmount | Number | Total transaction amount |
| TotalNumberOfItems | Integer | Total number of items in the order |
| CustomerXRef | String | Card Number or External Reference number |
| | | to uniquely identify the customer. |
| ClubNum | Integer | Unused |

Order.OrderItems

| | | |
|---|---|---|
| SequenceNum | Integer | Product order entry sequence |
| DetailCancelFlag | Integer | Was the product cancelled |
| DetailReturnFlag | Integer | Was the product returned |
| DetailSubtractFlag | Integer | Was the product subtracted from order |
| EntryMethod | String | Method for product entered |
| | | [EntryScanned\|EntryKeyed] |
| UPC | String | Product entered - check digit and leading 0's |
| | | removed |
| MerchandiseHierarchy | Integer | Department of product entered |
| UnitSalePrice | Number | Sale price per unit of item |
| UnitCostPrice | Number | Cost price per unit of item(Not used) |
| ExtendedAmount | Number | Total item sale price |
| Weighted | Integer | Weighted Item Flag |
| Negative | Integer | Negative item entry flag |
| ManualPrice | Integer | Manually entered price |
| PriceOverridden | Integer | Item price overridden |
| Quantity | Integer | Quantity of item entered |
| TaxAmount | Number | Tax of item (default 0.00) |

Order.Coupons

| | | |
|---|---|---|
| SequenceNum | Integer | Coupon order entry sequence |
| DetailCancelFlag | Integer | Was the coupon cancelled |
| DetailReturnFlag | Integer | Was the coupon returned |
| DetailSubtractFlag | Integer | Was the coupon subtracted from order |
| CouponPLU | String | PLU/UPCs used to identity coupon |
| CouponType | String | [STORE \| MFR] |
| Quantity | Integer | Number of coupons entered |
| Amount | Money | Amount of coupon savings |
| TenderTypeCode | Integer | Tender number |
| DepartmentNum | Integer | Department for coupon |
| NetoExcluded | Integer | |

Order.Tenders.Tender

| | | |
|---|---|---|
| SequenceNum | Integer | Coupon order entry sequence |
| DetailCancelFlag | Integer | Was the tender cancelled |
| DetailReturnFlag | Integer | Whether the order is a return |
| DetailSubtractFlag | Integer | Was the tender subtracted from order |
| EntryMethod | String | Method for tender entered |
| | | [EntryScanned\|EntryKeyed] |
| TenderNum | Integer | See TenderTypes in TABLE 2 |
| Amount | Number | Required tendered amount including tax |
| | | excluding coupon discounts |
| ChangeFlag | Integer | Was change returned |

Order.Taxs

| | | |
|---|---|---|
| TicketTaxNum | Integer | Default 1 |
| TicketTaxAmount | Number | Total tax on order |

Order.POSReceipts.POSReceipt

| | | |
|---|---|---|
| TextData | String | Digital Receipt |
| TextAttributes | String | Printer commands for receipt |

Table 2 is a list of tender types that are available for use in one embodiment. Each tender type is designated by a corresponding tender number.

TABLE 2

| Tender Number | Tender Type |
|---|---|
| 1 | CASH |
| 2 | CHECK |
| 3 | OTHER CHECK |
| 4 | WIC CHECK |
| 5 | CREDIT |
| 6 | GIFT CARD |
| 7 | DEBIT CARD |
| 8 | OFFLINE EBT VCHR |
| 9 | EBT FS |
| 10 | EBT CASH |
| 11 | CANADIAN EXCHNGE |
| 12 | CANADIAN TRAV CK |
| 13 | VENDOR COUPON |
| 14 | STORE COUPON |
| 15 | COIN STAR |
| 16 | AR CHARGE |
| 17 | WEB ORDERS |
| 18 | 3RD PARY PAYMNT |
| 19 | MISC TENDER |
| 20 | LOTTO PAYOUT |
| 21 | SCRATCH PAYOUT |

Table 3 is a product item file data dictionary for use in one embodiment. The product item file may be embodied as a pipe (“|”) delimited ASCII file that contains the fields shown in Table 3.

TABLE 3

| Field# | Field Name | Type | Required | Description |
|---|---|---|---|---|
| 1 | Program | String(4) | Yes | Program number to uniquely identify |
| | Number | | | program - left 0 padded |
| 2 | Merchant | String(4) | No | Merchant number to uniquely identify |
| | Number | | | merchant - let 0 padded |
| 3 | Store Number | Integer | No | Store number of product item file |
| 4 | UPC | String | Yes | Product UPC - trim leading 0's and check |
| | | | | digit |
| 5 | Description | String | No | Description used at the point of sale |
| 6 | Department | Integer | Yes | Department associated with product upc |
| | Num | | | |
| 7 | Brand | String | No | Brand associated with product |
| 8 | Manufacturer | String | No | Manufacturer of product |
| 9 | StatusCode | String(2) | Yes | AC—Active, available at stores |
| | | | | IN—Inactive, currently not available |
| | | | | DI—Disabled, no longer used |
| 10 | Level1 | String | No | First level of product categorization |
| | Category | | | |
| 11 | Level2 | String | No | Second level of product categorization |
| | Category | | | |
| 12 | Level3 | String | No | Third level of product categorization |
| | Category | | | |
| 13 | PAC | String | No | Price association code |
| 14 | Price | Money | Yes | Unit sales price of product |
| 15 | Unit | Integer | Yes | Unit count of product |
| 16 | Unit of | String | No | Unit of measure of product |
| | Measure | | | |
| 17 | AisleLocation | String | No | Aisle location of product |
| 18 | AisleSide | String | No | Aisle side [a or b] |
| 19 | AisleSection | String | No | Section within Aisle |
| 20 | AisleShelf | String | No | Shelf within Aisle |

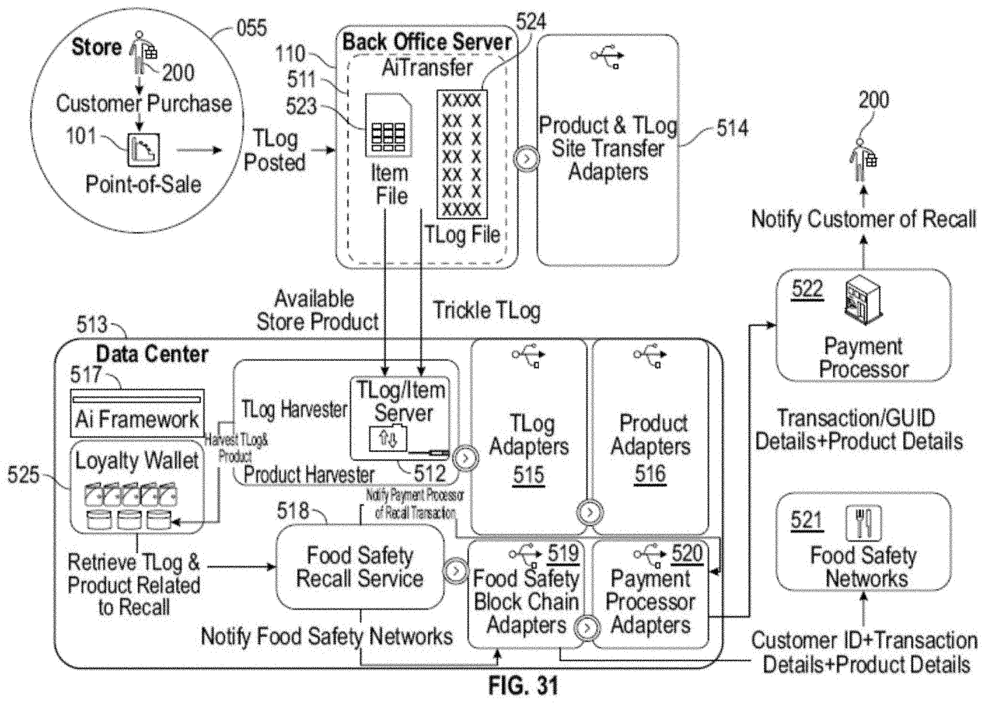

FIG. 31 illustrates an embodiment of the system and its store services architecture. A customer 200 makes a purchase at store 055. The purchase is completed using POS device 101, which collects customer and transaction information. The POS device 101 then posts a TLog file to back office server 110, which may be located at store 055 or in some remote location. Back officer server 110 runs a TLog transfer service (AiTransfer) 511, which is responsible for sending TLog files and product item files to a server 512 in cloud datacenter 102. AiTransfer service 511 understands each POS system 101 and loads appropriate product and TLog site transfer adapters 514 to properly send trickle feed TLog files and product items files to server 512. The product item file describes the products that are available to the POS system. The TLog file is the order file created by native POS systems at the completion of customer checkout actions.

Site transfer adapters 514 are software components that are created for each supported type of POS. TLog site transfer adapters are responsible for making POS TLog data available to the system. TLog site transfer adapters know the specific POS details of how and where TLog data is made available and properly prepares the TLog data for transfer to datacenter 102. Product item site transfer adapters are responsible for making POS product item data available to the system. Product item site transfer adapters know the specific POS details of how and where product item data can be retrieved and made available for proper transfer to the datacenter 102.

Server 512 is configured for storage of store TLog and product item files. The server 512 is structured to intelligently organize data based on retail space definitions to optimally process all files. TLog and product harvesters are services used to properly process POS-specific TLog files and product items files. The TLog harvester is the main processing engine for formatting different TLog formats into a standard system language format and properly storing them. The TLog harvester loads the appropriate TLog adapter 515 to properly process TLog files. Similarly, the product harvester is the main processing engine for formatting different product item files into standard system language format and storing them. The product harvester loads the appropriate product adapter 516 to properly process product item files.

Datacenter 102 further comprises an Ai Framework 517, which is a series of assemblies that encapsulate the full functionality of the food safety tracking system. The assemblies are agnostic to any display technologies and are responsible for the execution of standard data transactions and business rules that run the system. The Ai Framework 517 is an important part of the system and reflects a mental model of the problem space.

Food safety recall service 518 is configured to respond to recall alerts. Food safety recall service 518 retrieves TLog files and product information and makes it available to food supply networks and payment processors. Food safety recall service 518 ensures that customers 200 are properly notified of food safety recalls. Food safety recall service 518 loads load the appropriate food safety blockchain adapter 519 and payment processor adapter 520 for the food safety network 521 and payment processor 522 associated with a recall transaction.

Food safety blockchain adapters 519 are provided for each of the food safety blockchain networks 521 that works with the system. Food safety blockchain adapters 519 are responsible for properly filtering available data to meet the requirements of a food safety network 521. The food safety blockchain adapters 519 utilize the interface for a specific food safety network 521 to send TLog and product information to its blockchain. This data contains no PII.

Payment processor adapters 520 are provided for the various payment processors 522 or banks that need TLog order information to link a transaction to a consumer. The payment processor adapters 520 contain no PII information and strictly contain logic to map transactional data to a processor's API.

Food safety networks 521 represent the various food safety networks, such as IBM Food Trust. Payment processor 522 may be a bank or payment processor that works with the system for food safety initiatives. Communication with the payment processor 522 allows the processor to link transactions to customers 200 for food recall notifications.

The following sequence of events describe the workflow required for properly handing food safety recall notifications. A customer 200 makes a purchase on a standard store-based POS system 101. The POS system posts TLog files to its back-office server 110. A product item file 523 is built from the POS system data to reflect the latest available products within store 055. Product item file 523 is sent to TLog/Item server 512. At periodic intervals, TLog files 524 that reflect POS transactions are also transferred to server 512.

TLog files and store product items are processed and stored within the system, such as in a loyalty wallet storage 525. TLog and product item files that are related to a product recall are retrieved by food safety recall service 518, which determines when recalls occur and retrieves order and product information for orders related to the product recall. The food safety recall service 518 determines from an order what payment processor 522 should be notified of the product recall. Food safety recall service 518 loads the proper processor adapter 520 to communicate with the payment processor 522. Transaction and product details related to the recall are sent to the payment processor 522. A particular transaction or order may be identified by a GUID. Payment processor 522 may notify the customers 200 who are associated with the transaction about the product recall.

Food safety recall service 518 understands what brands and retailers need to have transaction and product information published to a food safety network 521. Food safety recall service 518 loads the proper food safety adapter 519 to communicate properly to a specific food safety network 521. The food safety adapter 518 aggregates a non-PII customer identifier (e.g., a GUID), transaction details, and product details, and then writes this information to food safety blockchain network 521 using adapter 519.

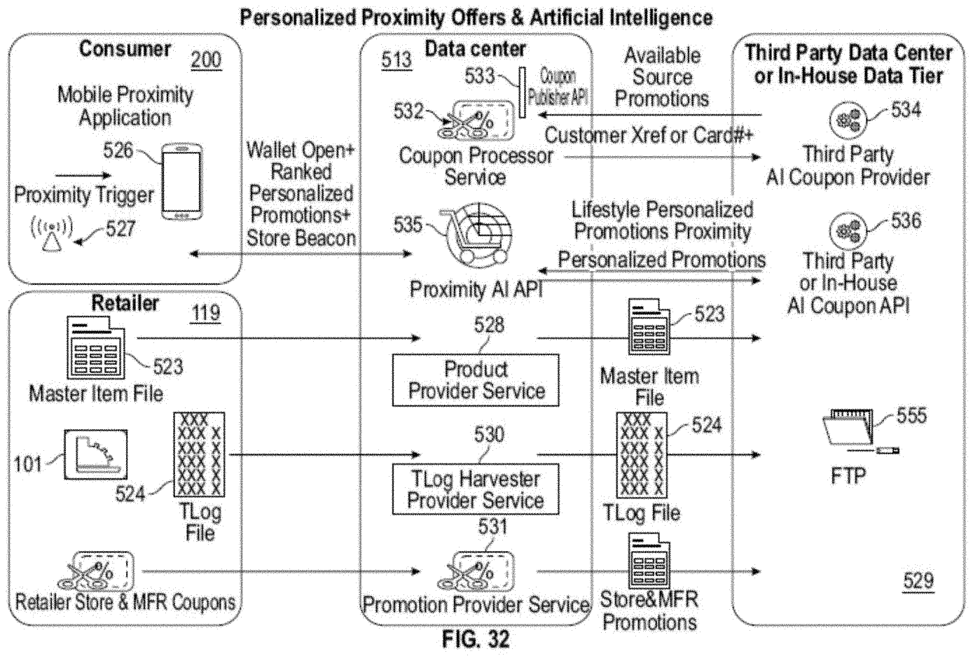

FIG. 32 depicts an embodiment of the system, architecture, services, interfaces, and data flows that represent the support for personalized lifestyle and proximity-based offers. A consumer 200 has a mobile device 526. A mobile proximity application on the mobile device 526 detects location signals 527 and, based on the device 526, displays available offers for clip and redemption. The location signals 527 may include a proximity trigger or other signal that identifies a location. The location signals 527 may be broadcast by a pico cell or other short-range transmitter, GPS, or other sensor methods. The mobile proximity application may be used to provide in-store, location-based offers. The consumer's mobile device 526 exchanges information with datacenter 513, such as identifying an account or digital wallet, a ranking of personalized promotions, and the received store beacon or proximity trigger.

A retailer 119 provides information, such as a master item file 523 and TLog file 524 to datacenter 513, that is collected when consumer 200 makes purchases at POS 101. A product provider service 528 provides in-store product information including location mapping within a store to third-party or in-house AI system 529. A TLog harvester provider service 530 provides TLog files 524 to the third-party or in-house AI system 529. A promotion provider service 531 in datacenter 513 may be used to provide store-based offers (e.g., store-based or manufacturer coupons or promotions) to the third-party or in-house AI system 529. The item file, TLog file, and store promotion data may be stored using FTP 555 in the third-party or in-house AI system 529. FTP 555 may be configured to store large amounts of information that is used by the AI engine to generate lifestyle and location-based offers.

Algorithms in the third-party or in-house AI system 529 may use the item file and TLog information to calculate the best offers to present to consumer 200 based on the consumer's location within the store 119 as indicated by proximity trigger or GPS 527. The store and manufacturer promotion information may be used to ensure that duplicate offers are not presented to customers based on location within the store.

Coupon processor service 532 is configured to process incoming third-party offers, translate the offers to a required format, and make the offers available to subscribing retailers 119 and consumers 200. The coupon processor service 532 may provide offers to be used for both proximity and non-proximity-based services. Coupon publisher API 533 is a RESTful API that allows third parties to publish digital offers and coupons into specific merchant channels. The API 533 also allows third parties to link coupons to a customer's specific merchant loyalty account or wallet based, for example, on a customer reference number or loyalty card number.

Proximity AI API 535 is a RESTful API that is used by in-house or third-party mobile applications to present offers based on location within a store 119. API 536 uses artificial intelligence to determine a best offer to present to customer 200 based on location within store 119. A third-party AI coupon provider 536 makes offers available to retailers 119. AI coupon API 536 is used to support both the presentation of lifestyle and location-based offers. These APIs 535, 536 can be used in real time to present the best offers to consumer 200. For example, offers may comprise a unique customized incentive based on current time, customer location, past purchases, etc.

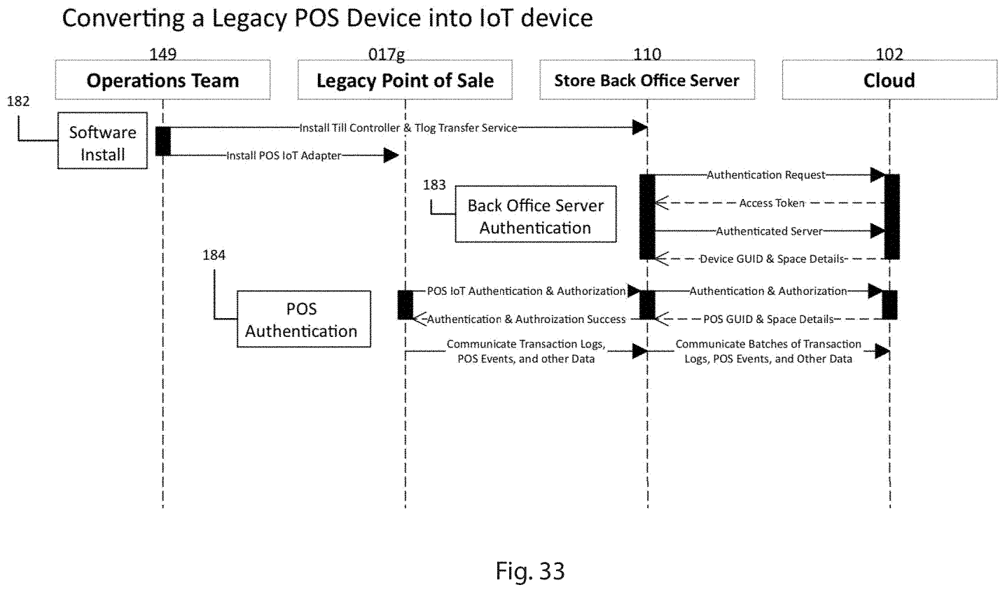

FIG. 33 depicts a method for conversion of legacy POS systems to IoT devices that are capable of communicating with the cloud. An operations team 149 installs software 182 on the legacy POS device 017g and store back office server 110. The legacy POS devices 017 g are installed with a POS IoT adapter and the store back office server 110 is installed with a till controller and TLog transfer service. With this new software, the store back office server 110 authenticated 183 with the cloud 102 by: sending an authentication request, receiving an access token, using this token to get authenticated, and then receives a GUID and space details from the cloud 102. This is where a digital representation of the POS device 152 is assigned a POS device 101 and the space details contain information on how this back-office server 110 and legacy POS devices fit into the larger enterprise ecosystem (within the system 130). Next, the legacy POS 017g authenticates 184 with the cloud 102 through the store back office server 110. First, the POS devices leverages the newly installed software 182 to request authentication and authorization from the Store Back Office Server 110. The back-office server 110 then relays that request to the cloud 102, which confirms and returns a POS GUID and space details for the POS device. This is held within the store back office server 110, that confirms with the POS device 017g that it has been authenticated and is authorized to use services. The POS device 017g can now communicate transaction logs, POS events, and other data with the store back office Server 110. The back-office server 110 batches communications from multiple POS devices it is in communication with and communicates that information (transaction logs, POS events, and other data) with the cloud 102.

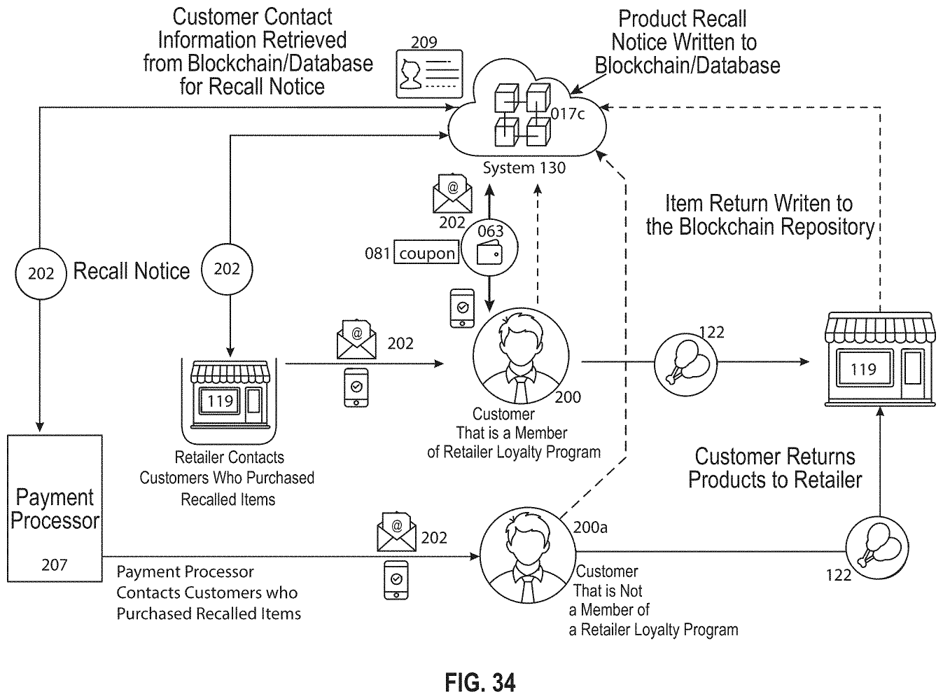

FIG. 34 depicts interactions between customers 200, 200a, retailers 119, and payment processors 207 within system 130 when a product recall occurs. Customer 200 is a member of a loyalty program for retailer 119. When customer 200 makes a purchase at retailer 119, the transaction is recorded to a blockchain repository 017c in the system 130. In other embodiments, any repository may be used in place of a blockchain 017c, such as a database or table. The transaction information may include a customer cross reference or loyalty account number, for example, that can be used to link customer 200 to the transaction. In other embodiments customer 200 may not want to sign up for a loyalty program but gives a telephone number to be contacted in case of a recall. Customer 200a is not a member of the loyalty program for retailer 119 or has not identified any loyalty program identifier when making a transaction at retailer 119. When customer 200a makes a purchase at retailer 119, his or her transaction data is also recorded to blockchain repository 017c (or other repository) in the system 130. The transaction information may include a unique identifier for the payment instrument 209 that customer 200a used to complete the transaction, which may be, for example, a credit or debit card associated with payment processor 207. In some embodiment the identifier for the customer 200a may be a series of values (e.g. transaction number, POS number, date, store number, etc.) In some embodiments, transaction information for customer 200 may also include a unique payment processor identifier in addition a loyalty account number.

The system 130 may become aware of a product recall, for example, when notified by a retailer 119, brand, manufacturer, distributor and/or government agency. The product recall notice is written to the blockchain and/or database 017c. System 130 may issue a recall notice directly to customer 200 if system 130 has contact information for customer 200. The recall notice 202 may be sent by any method, such as a text, postal mail, email, telephone call, online posting, or notification in a digital wallet or application. System 130 may also send a coupon 081, store credit, or other compensation to customer 200 such as if authorized by retailer 119 or the manufacturer to compensate consumers for the recall. A physical coupon 081 or other compensation may be sent to customer 200 by postal mail, or the compensation may be added electronically to a digital wallet 063, mobile application, or other account or may be made available online.

System 130 may issue a recall notice 202 to retailers 119 that sold the recalled product. Retailer 119 then contacts customers 200 who purchased the recalled product. Retailer 119 knows who customer 200 is based on a loyalty card account or other identifier that was recorded during the initial purchase transaction. The recall notice 202 may be sent by any method, such as a text, postal mail, email, telephone call, online posting, notification in a digital wallet or application, or printed on transaction receipts.

System 130 may issue additional recall notices 202 to payment processors 207 that are associated with a payment instrument 209 that was used to purchase the recalled product. Payment processor 207 then contacts customers 200a who purchased the recalled product. Payment processor 207 knows who customer 200a is based, for example, on the unique transaction identifier (or series of values) that was recorded during the initial purchase. The recall notice 202 may be sent by any means, such as a text, postal mail, email, telephone call, online posting, or notification in an account statement.

Once the customers 200, 200a receive notice of the recall, they can return the recalled product to the retailer 119 or dispose of the product, as appropriate for the circumstances of the recall. When retail 119 receives returned recalled products, the item's return is written to the blockchain repository (or other repository) 017c in system 130.

The process illustrated in FIG. 34 enable retailers 119 to immediately stop purchases of recalled products at the POS. For example, if a customer 200 is in line to purchase a recalled product and a recall is entered into the system 130, then the POS will not ring up the recalled item and the customer 200 would be notified that the product has been recalled.

Individual customers 200, 200a may sign up for a food safety initiative by giving their telephone or mobile number at the time of purchase, which would link the customer's contact information to the transaction record in blockchain repository (or other repository) 017c. Customers who purchased recalled items may then receive a text message that includes a link with further information about the recall, such as a link to the CDC, FDA, or the brand or manufacturer. Recall information may also be written to a purchase receipt. Customers 200, 200a may also receive an immediate coupon for their trouble of having purchased a recalled product as a gesture of goodwill from the brand or manufacturer.

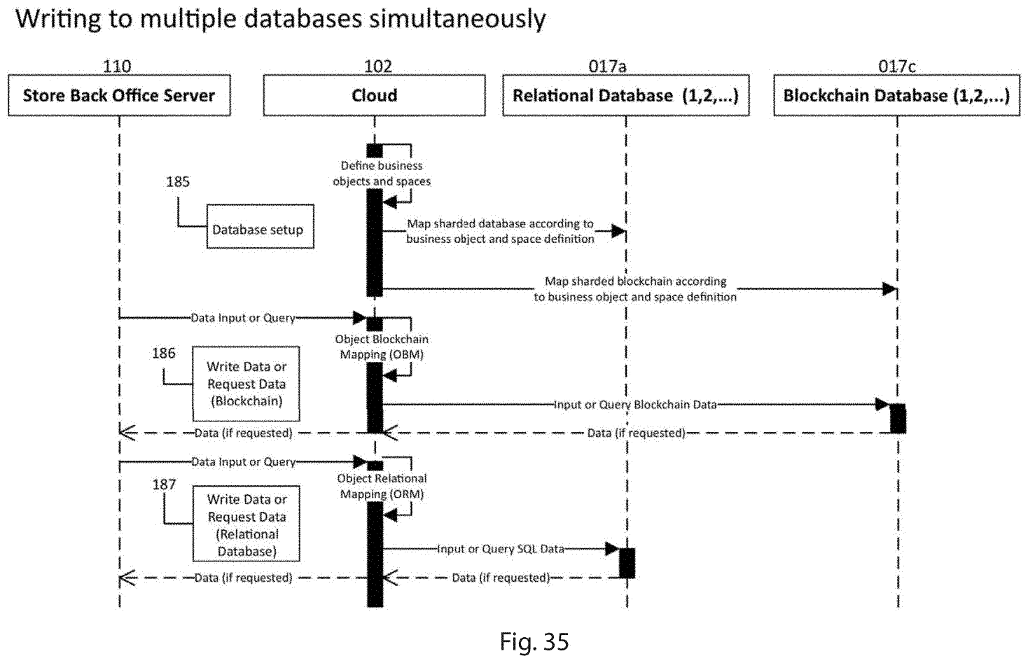

FIG. 35 depicts a method for writing to multiple databases simultaneously. The cloud 102 first defines all business objects and spaces to set up the databases 185. This is the process of representing and relating all business objects into a digital form. This can include, but not limited to, the stores, brands, promotions, and consumers. There is a representation of these objects and their relationships to one another. These relationships are mapped and documented within the relational databases 017a and blockchain databases 017c. This setup 185 takes place across multiple databases 017a and 017c to ensure the ability to shard and scale appropriately. Whenever a store back office server 110 signals to write data or request data to the blockchain 186, the cloud 102 references the maps created in the database setup 185 to identify on which blockchain database 017c, and where within that database, the data should be written or requested. If data is requested, that data is then returned to the cloud 102, which then sends it to the appropriate location (store back office server 110 in this instance). The process is similar for relational databases 017a ORM. When the store back office server 110 signals to write data or request data 187 from a relational database 017a, first the cloud 102 references the relational map created during the database setup 185 to identify the correct relational database 017a and location within that relational database 017a. If data is requested, that is then returned to the cloud 102 that then transfers the data to the appropriate location, the store back office server 110 in this instance.

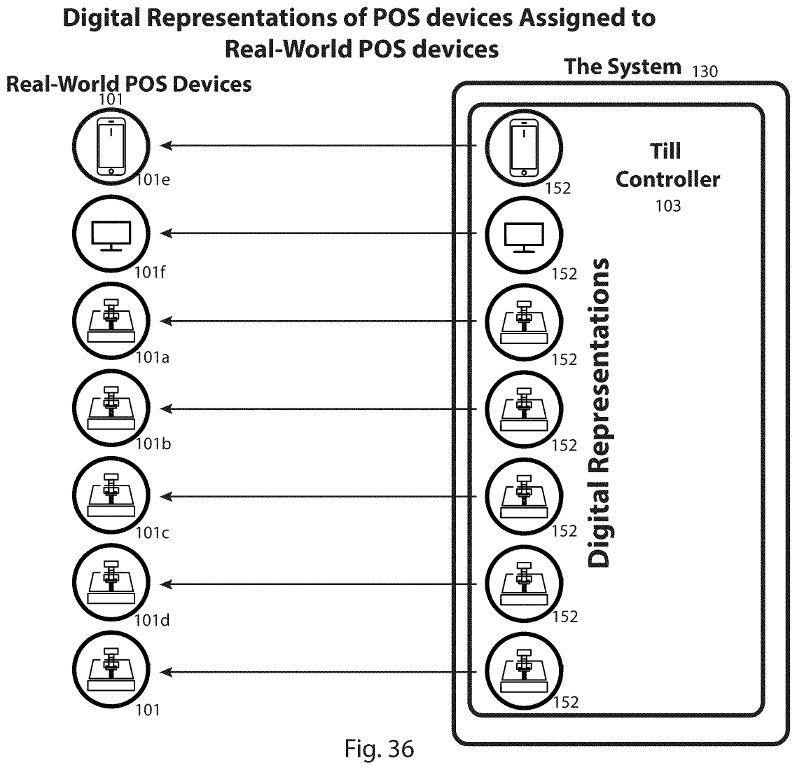

FIG. 36 illustrates an embodiment of the system where digital representations are being assigned to real world devices by the system. In order to interact with the system 130 (and become part of the system), real world devices are assigned a digital representation 152 of the real-world device. Depicted are mobile device 101e, online device 101f, ISS45 POS 101a, IBM POS 101b, RORC POS 101c, ScanMaster POS 101d, and any other POS device imaginable 101 (virtual or physical). Till controller 103 within the system 130 contains/manages the digital representations of POS devices. The store till controller (or till controller in the cloud 102) is a POS-IoT Hub that creates and manages all digital representations of POS devices within the store (and otherwise). Till controller 103 that is found on the back-office server 110 in a retail store 119 or on a server 026 in the cloud 102. The till controller 103 assigns each of these POS devices that interact with the system 130 a digital representation 152 of their real-world counterpart. Each digital replica 152 would include at least a GUID or unique identifier. By bringing the real-world points of sale 101 within the system 130, the system is able to manage, track activity and interact with each POS. Real world POS systems 101 can also receive added functionality through their digital representation (e.g., becoming IoT compatible, etc.). Because of the assignment of digital representations, the real-world POS devices are now able to interact with the system 130 and any other system component necessary.

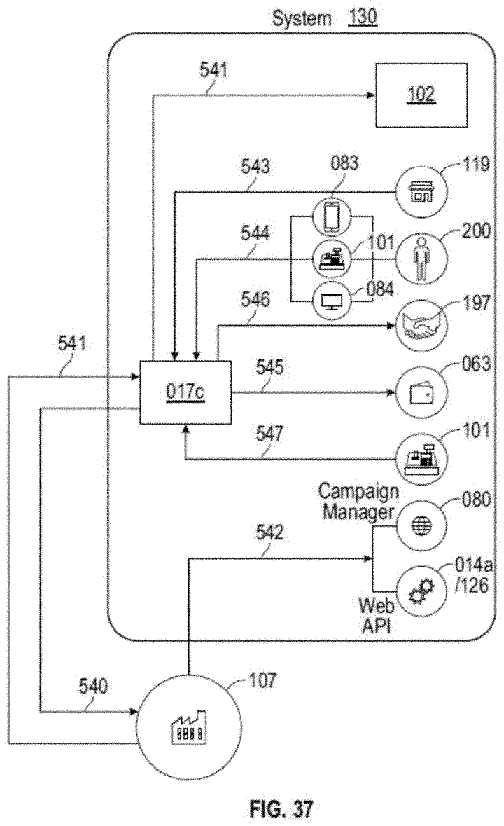

FIG. 37 illustrates how a brand 107 might interact with the system 130 according to one embodiment. The brand 107 registers 541 with the network 102 via repository 017c, which may be a blockchain repository. The system 130 provides a campaign manager 080 and webservices 014a /API 126 that allows the brand 107 to interact 542 with system 130. Campaign manager 080 may be logic or a software engine that manages campaigns, such as to add campaigns, promotions, Adcentives (or other incentives), daily deal content, third-party rewards, UPCs for recall tracking, supply chain information, and e-TPRs (electronic temporary price reductions) and to set campaign budgets and thresholds for participating retailers. Information may be sent 540 to the brand 107 over blockchain and uses smart contracts for promotions or other manner (e.g. API).

A retailer 119 may request 543 to participate in brand 107's network. Consumers 200 at a retailer 119 location may also request 544 to register with brand 107. Such consumer requests may be forwarded 540 over blockchain from a mobile device 083, online communication 084, or POS 101, for example.

Consumers 200 may have elected to share data when registering 544. The system 130 requests 540 brand-targeted content over blockchain for consumers 200 who choose to share data. Brand 107 may send 545 personalized benefits to the consumer's wallet 063 over blockchain 017c, API or other manner. The benefits may be based on shopping history, shopping list use, or ad viewing, for example. Brand 107 may create 542 a reward offer that includes a third-party partner, such as another vendor who provides fuel points, airline miles, movies or services as a reward when a brand product is purchased. Brand 107 sends a request 546 over blockchain (or API, etc.) to a reward partner 197 to participate in a third-party reward offer.

Brand 107 may accept transaction data 547 from retailer 119 over blockchain, such as from a retailer's POS 101, or other manner. In the event of a recall, brand 107 notifies 541 system 130 of the recalled product. The system 130 then notifies consumers 200 of the recall via their preferred method of communication, such as via a mobile device 083 (e.g., call, text, application notice) or online communication 084. Alternatively, a POS 101 may write recall information on a receipt, such a number to call or website to visit for future recalls. The POS 101 may print a recall notice on a later receipt for a customer 200 who made an earlier purchase of a now recalled product (i.e., warn the customer 200 of the recall on a later visit). In other embodiments the system may notify a payment processor who is affiliated with the payment instrument used to purchase the product who then notifies the customer or the retailer may be notified so that they can notify their loyalty members directly.

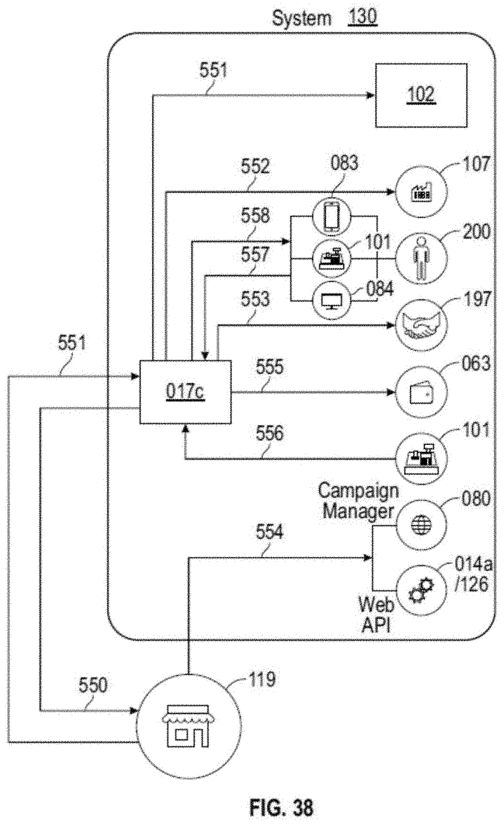

FIG. 38 illustrates how a retailer 119 might interact with the system 130 according to one embodiment. The retailer 119 registers 551 with the network 102 via repository 017c, which may be a blockchain. Responses 550 to retailer 119 may be send via blockchain. The retailer 119 may denote data points that are needed and whether it has an existing loyalty system, phone number, or other information. The retailer 119 can set rules on how partners will share data. The retailer 119 may elect to reward consumers for registration. The retailer 119 may designate a preferred consumer recall communication method. System 130 including cloud datacenter 102 may provide adapters that convert the retailer's legacy POS systems 101 into IoT devices for use with the system 130.

Once registered, retailer 119 may request 552 to participate in brand's 107 network. Retailer 119 may also request 553 to work with participating third-party reward partners 197 over blockchain. System 130 provides retailer 119 with a campaign manager 080 and webservices 014a/API 126 that allows the brand 107 to interact 554 with system 130. Campaign manager 080 may be a piece of logic or engine that that manages campaigns, such as to distribute retailer promotion content (e.g., Ads, or Adcentives). The retailer 119 may configure post-transaction rewards partners 197, such as fuel rewards, airline miles, or other third-party participants in the retailer's campaign. Retailer 119 may add daily deals for publication, for example. Retailer 119 may accept a partner ad request to be included in a consumer app display and may accept product recall notifications.

Retailer 119 and other partners 197 send 555 personalized benefits to the customer's wallet 063 over blockchain. Retailer 119 may accept 556 transaction data, requests 557 to participate in the retailer network, and till monitor data elements 556 over blockchain from real-time customer checkout information received from POS 101 or a customer device.

The retailer 119 may generate product recall alerts 558 to the customer 101 as products scan. The system 130 may notify consumers 200 of the recall via their preferred method of communication, such as via a mobile device 083 (e.g., call, text, application notice) or online communication 084. Alternatively, a POS 101 may write recall information on a receipt.

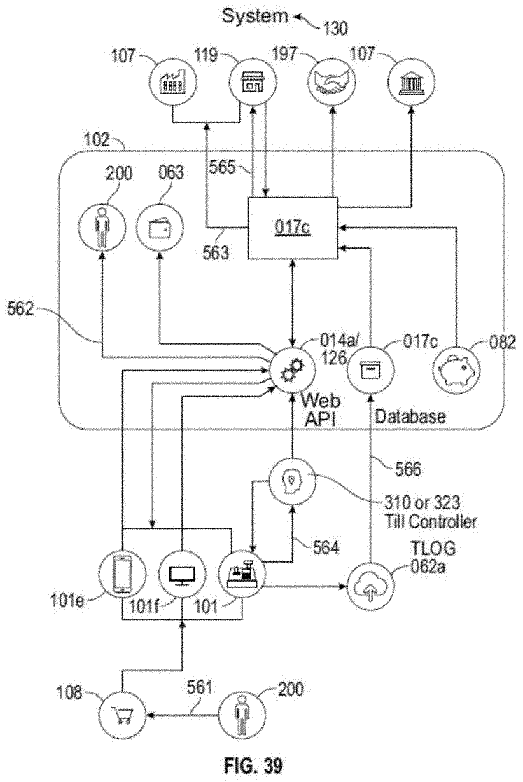

FIG. 39 illustrates customer 200 interactions with a retailer 119 and/or brand 107 using system 130 according to one embodiment. Retailer 119 is registered with network 102 via repository 017c, which may be a blockchain. Retailer 119 is registered to participate in brand 107's network using network 102.

Customer 200 registers 561 for retailer 119's loyalty program and request recall notifications. This registration can occur, for example, during a purchase transaction 108 that is completed using a through mobile POS 101e, online POS 101f, or in-store POS 101. The registration is handled by webservices 014a/API 126 in network 102 (cloud). Customer 200 may be sent 562 an SMS or other message to download a mobile app to begin the retailer or brand relationship. Customer 200 may choose how to share their data with brand 107 and other partners 197, including other retailers 119 participating on system 130.

Customer 200's registration is sent 563 to retailer 119 and/or brand 107 over blockchain or other manner (e.g. API, etc.). Customer 200 may be rewarded by brand 107 and participating partners 197 for sharing data. The reward may be, for example, a coupon, store credit, store points, or cryptocurrency that is credited to the customer's digital wallet 063. Customer 200's registration may occur using the customer credentials on network 102 for another retailer or brand program (i.e., for an existing registration).

Customer 200 shops at a participating retailer 110 either in-store 101 or online 101e, 101f. The customer 200 can use a mobile checkout app to add items to their basket or cart. Customer 200 identifies himself or herself at a register, mobile checkout app, or online.

The customers digital wallet 063 is opened 564 via a store till controller 310, 323. Wallet 063 contains the customer's segment, store credit, points and available promotional benefits, and payment accounts, including cryptocurrency, to be applied during checkout.

During checkout, each item in the basket is sent 564 to the store till controller 310, 323 for promotion and recall safety analysis. Each scan is captured by the till monitor 310, 323 and sent to subscribers to monitor real-time data over blockchain (or other method, such as an API). Before tender of payment, brand and retailer promotion benefits are added to the order or transaction. Customer 200 completes a purchase at POS 101 or with mobile checkout app 101e, 101f. The tender choice occurs through wallet 063 payments including cryptocurrency. Coupons, fiat currencies, and/or multi-tender.

The transaction is captured within system 130 through the POS IoT. Redeemed promotion benefits are sent 565 to retailer 119 over blockchain 017c. Redeemed promotion benefits are sent to brand 107 over blockchain 017c and other subscribers are notified. Customer 200 receives 562 an SMS and/or email message with an e-receipt for the transaction.

Subscribed transaction log (TLog) data points 062a are sent 566 over blockchain (or via other manner) to subscribers including brand 107, retailer 119, and participating partners 197 over blockchain 017c (or API or other method). Brand 107 and retailer 119 are notified of any benefit redemption. Promotion benefits are settled and cryptocurrency (or digital currency or other tender) payment may be exchanged from brand 107 to retailer 119 over blockchain (or other manner). Registered retailer EFT/ACH accounts 107 are settled based on brand promotion value.

Post-transaction rewards are determined, and rewards, such as fuel points or airline miles, are sent over blockchain, API (or other manner). Other third-party-rewards subscribers are credited over blockchain, API or other manner. Appropriate adjustments are made to the accounts or promotions based on third-party rewards.

During transactions, geolocation, such as a proximity beacon, is used to acknowledge customer 200 arrival at a retail location and notification is sent over blockchain. Proximity offers may be presented to the customer 200 depending on customer location in the store.

Brand 107 (or government agency) notifies the system if a recall occurs. The system 130 identifies all customers 200 that purchased the recalled product. It stops purchases of the product at the Points of Sale within participating merchants. By blocking all recalled product from being purchase and sending notification to the POS for explanation as to why sale was stopped. The customer's bank 207 (or payment processor associated with the payment instrument used to purchase the now recalled product) or the customer 200 is directly notified of the product recall by the system 130. The customer may be offered a brand 107 promotion or coupon in response to the recall.

In some embodiments, repository 017c is a database and transaction, customer, retailer, and brand data goes directly into the repository 017c wherein the data is stored. Alternatively, select portions or all of the data may then go into a blockchain repository 017c. The opposite may also occur, wherein data stored to a blockchain repository 017c would also go to a separate database 017c.

A user account 082 may be used in place of digital wallet 063 if a customer wanted to sign up for a recall account (i.e., if the customer wants to receive recall information) but does not want a digital wallet 063. The customer 200 could provide his or her phone number and, if they purchase a product that is recalled, system 130 would notify the customer even if he or she does not have a digital wallet.

FIG. 40 depicts a high-level view of a system 130 for the tracking of consumer products 118 and for the implementation of a recall 202 via the use of a blockchain 017c and/or other secure database.

The system 130 may include a POS device 101. The POS device 101, discussed in more detail herein, may be part of a merchant system or otherwise associated with a merchant 119 and used to initiate electronic payment transactions for processing, including the purchasing of products. The POS device 101 may be any type of traditional POS device that is specially configured to perform the functions discussed herein, such as through specialized hardware and software configuration thereof. For instance, the POS device 101 may be a specially configured desktop computer or tablet computer that is configured to act as a POS or may be a virtual POS where a customer 200 walks out of a store and the products the customer purchases are automatically charged to the customer's account via the payment instrument or other type of POS using the methods discussed herein.

In the system 130, a customer 200 purchases a product 118 via a POS device using a payment instrument as defined herein. The customer purchases a product 118 as part of an electronic payment transaction 211. The customer 200 receives the product 118 from a merchant 119 associated with the POS 101. The product 118 is associated with a brand 107 and is assigned a unique product identifier 181 by the brand 107. The POS 101 propagates a transaction log that contains at least a unique product identifier 181 and payment transaction data 062a. The payment transaction data 062a may contain a transaction number, transaction amount, POS number, store number, date, and/or other identifying information which make up a customer GUID 117. A customer GUID 117 helps identify a customer without the necessity of personal identifying information (e.g., name, address, phone number). The product identifier 181 along with the customer GUID 117 are posted to the blockchain 017c or other secure database. The product identifier 181 along with the customer GUID 117 are posted in such a manner that it can later be determined that a customer 200 with a customer GUID 117 purchased a product 118 with a product identifier 181. The blockchain network 017c may be a network of a plurality of computing nodes 112 (also referred to herein as “blockchain nodes”) that is configured to store and manage a blockchain, including the generation, verification, and addition of new blocks thereto.

The blockchain may be comprised of a plurality of blocks. Each block may be comprised of a block header and a plurality of transaction values. The block header in a block may be comprised of at least a reference to a previous block, a timestamp when the respective block was generated, and a reference to the plurality of transaction values included in the respective block. In an exemplary embodiment, the references included in a blockchain may be hash values generated via the application of one or more hashing algorithms to the associated data. For instance, the reference to the previous block may be a hash value generated via the application of a hashing algorithm to the block header of an earlier block in the blockchain. The use of references may ensure the immutability of the blockchain, as a change to any transaction value in the blockchain would yield a different reference value, requiring the corresponding block header to be changed, which in turn would yield a different reference value for that block header, requiring every single corresponding block to be changed. As such, every block in the blockchain may be verifiable by the calculation of the reference values using the appropriate hashing algorithms.

In certain embodiments when a product 118 is purchased by a customer 200, data 062a for the transaction may be electronically transmitted to a node in the blockchain network 017c for addition to the blockchain. The transaction data 062a may be accompanied by an identification value (or values) 117 associated with the customer 200 identification value (or values) 181 associated with the product 118. In one embodiment, the identification value may be a blockchain address associated with the customer 200 or an address associated with a brand 107. In another embodiment, the identification value may be a digital signature generated by the customer 200 and supplied to the POS device 101. The blockchain node 112 may add select transaction data 062a to a new block to be added as one of the plurality of transaction values. The block may be verified by other nodes 112 in the blockchain network 017c using traditional methods and systems and then added to the blockchain.

The customer 200 may possess a payment instrument 209 that may be configured to convey data to the POS device 101 to authenticate the individual's eligibility to pay with the payment instrument 209. The payment instrument 209 may be any type of payment instrument that may be issued to an customer 200 for use in conveying data to a POS device 101, including the data discussed herein and payment credentials associated with a transaction account, such as a magnetic stripe card, integrated circuit card, computing device with an electronic wallet application program, etc. In the system 130, a payment processor 207 may issue a transaction account to the customer 200 for use in funding electronic payment transactions, for which the payment processor 207 may issue the payment instrument 209 to the customer 200. The payment processor 207 may be any type of entity configured to issue transaction accounts to a customer 200, such as a financial institution (e.g., an issuing bank, credit card company, etc.).

In certain embodiments, the payment instrument 209 may thus store the payment credentials associated with the related transaction account as well as data corresponding to the identification value 117 stored with the product data 181 in the blockchain. In embodiments where the identification value may be a blockchain address, the payment instrument 209 may include the blockchain address. In some instances, the payment instrument 209 may include a private key of a key pair, where the private key may be used to generate the blockchain address. In such instances, the payment instrument 209 may convey the generated blockchain address to a POS device 101 (e.g., or to a suitable computing device of the issuing entity) for inclusion in the transaction value. In some such instances, the payment instrument 209 may also provide the public key corresponding to the private key in the key pair, which may be used by the POS device 101 (e.g., or other issuing entity) to verify the blockchain address.

In embodiments where the identification value may be a digital signature, the digital signature may be generated by the payment instrument 209, such as using a private key of a key pair, where the corresponding public key may be used for verification of the digital signature. In some cases, the digital signature may be generated by the POS device 101 (or issuing entity) via a private key, where the corresponding public key may be electronically transmitted to the payment instrument 209 using a suitable communication method for storage therein. Or the payment instrument 209 may not communicate with the blockchain at all and that any information regarding the payment instrument 209 found on the blockchain 017c or other secure database is placed there via the POS 101 and/or POS connected devices.

When a customer 200 wishes to pay for a product 118, the customer may present the payment instrument 209 to the POS device 101. The POS device 101 may be configured to read the payment credentials stored therein as well as the data corresponding to the identification value. The data may be read from the payment instrument 209 using any suitable method, such as the reading of data encoded in a magnetic stripe included in the payment instrument 209, receipt of the data via electronic transmission therefrom using NFC, reading of a machine-readable code displayed by the payment instrument 209 that is encoded with the data, etc. The payment credentials may include a transaction account number and any other data associated with the transaction account that is necessary for the processing of an electronic payment transaction funded by the transaction account, such as a name, expiration date, security code, etc.

The POS device 101 may receive the payment credentials and the data associated with the identification value, may receive the product 118 information and data associated with the product identifier 181. Data regarding the product 118 may be retrieved from another device connected to the POS 101 (e.g., server, database, etc.) and the product identifier 181 on the product itself. The POS device 101, and/or connected devices, may then communicate with the blockchain network 017c or other secure database using a suitable communication network and method to post and to retrieve the transaction data 062a stored therein.

When a brand 107 or a government agency issues a product recall, the payment processor 207 may be notified so that the payment processor 207 may in turn notify customers 200 that used a payment instrument 209 that is in the payment processor's network (or network processor's system) to purchase the now recalled product 118. In embodiments where the transaction data 062a is stored in a block chain 017c or other secure database the blockchain 017c or other secure database is queried in order to retrieve the transaction data 062a of every customer 200 that purchased the now recalled product 118. The transaction data 062a (listing who purchased the now recalled product 118) along with information regarding the recall is sent to the payment processor 207 who then notifies customers 200 in its network or system. The payment processor 207 may also provide information to the customer 200 on steps to take to mitigate any danger to the customer 200. In some instances, the payment processor may be authorized to issue a refund to the customer 200 for the purchase price of the now recalled product 118. In other instances, a customer 200 may need to return the product to the merchant 119 where the customer 200 purchased the product in order to receive a refund. In other instances, the payment processor may give the customer information on where to go for information on the recall (e.g., website or other electronic media). In some instances, a payment processor may send the customer 200 bonuses and/or rewards for the customer's 200 inconvenience of having purchased a now recalled product 118.

In embodiments where a smart contract is employed to notify the payment processor, transaction data 062a provided to the payment processor may contain a list of customer GUIDs 117 along with product purchase information (e.g., date of purchase, place of purchase, etc.) and information on the recall (e.g., reason for recall, steps to take, etc.)

In some embodiments, an API is employed to interact with the payment processor 207 in order to enable the payment processor to identify the customers 200 that purchased the now recalled product 118 so that the payment processor 207 may effectively communicate recall information to customers 200. An interface is used to connect the payment processor 207 to the system 130.

In some embodiments where a digital packet of data is sent to the payment processor, the digital packet of data may contain a list of customer GUIDs 117 along with product purchase information (e.g., date of purchase, place of purchase, etc.) and information on the recall (e.g., reason for recall, steps to take, etc.).

The customer GUID 117 may be a single value that the payment processor 207 may use to identify the customer 200 who purchased the product, or it may contain several pieces of data (e.g., place of purchase, POS number, transaction number, and/or date, etc.) that the payment processor 207 could use to determine the customer 200 identification

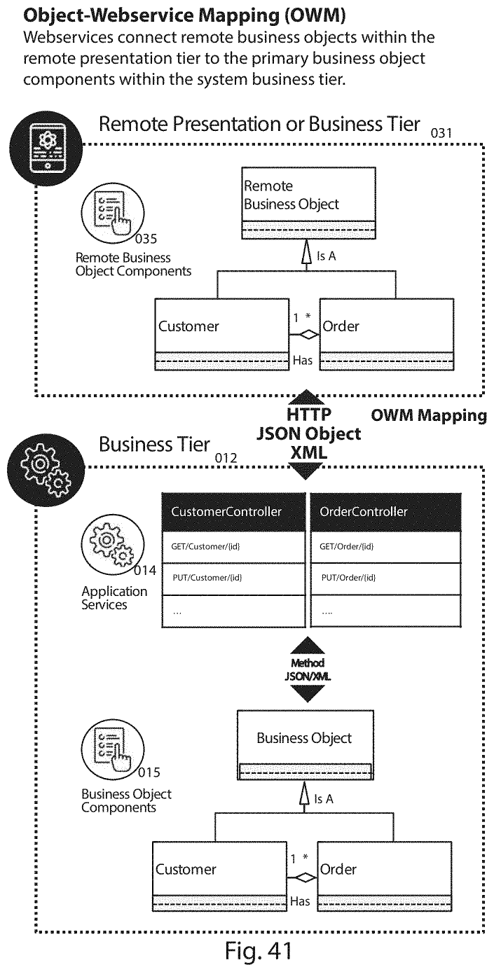

FIG. 41 depicts business objects that reside in remote client locations, including legacy hardware as well as mobile or handheld devices. The remote data technology webservice adapter 060h provides similar capabilities as the ORM adapter 060a used by business object components 015 that are mapped to relational databases. The webservice adapter 060h maps remote business object components 035n to the structure required by the business tier webservice 014a. Since the webservice 014a is designed to support the naming and structure of business objects 015, a common technique to interact with the webservice 014a is used. This technique is called OWM to reflect the similar concepts used by an ORM.

The OWM is designed to encapsulate the mapping of the standard remote business object 035 CRUD operations to the HTTP verbs Post, Get, Put, and Delete that support RESTful webservices. The business object 015 name is used as the URI resource name and actions become the verb. URIs are formatted as https://<host>/<object name>/{[ObjectID]}/[<action>|<aggregate/child name>].

When communicating with a web service 014a, data is transferred using a JSON object. The object is intelligently defined because it knows which properties are required to be sent, such as an ObjectID or modified properties. Aggregate or child JSON are included when it is required by the operation. In the case of a response containing a JSON object, it is deserialized into its associated business objects 015.

FIG. 42 depicts how business objects that reside in the cloud map to blockchain technologies. Within the system 130 object-oriented design focuses on objects rather than databases. The system 130 object-oriented designs develop the ability to seamlessly transition between any repository or database 017 (e.g., from SQL to blockchain, etc.). All business objects 015 within the system 130 may be developed as object-oriented solutions. It is similar to ORM, but with the transition to blockchain, it allows for an easy transition to OBM. System 130 database interactions can be updated from relational to blockchain (or to include blockchain).

Object design within the system requires diligence to ensure all applications adhere to the macro strategy of coding. Each object goes virtually untouched as new blockchain adaptors are added.

FIG. 42 shows the mapping of operations and data to a blockchain repository 017c. Business object operations are mapped to smart contract functions. The mapping algorithm contains the following steps:
Business object 015 is mapped to equivalent or similar named smart contract 048;
Business object operation is mapped to equivalent or similar smart contract 048 or smart contract function;
Business object properties are mapped to smart contract function parameters;
Business object properties are mapped based on (a) identity of blockchain network member and (b) smart contract function parameters (members subscribed view of data);
Business object 015 is mapped to identity of client (brand or retailer) 174 and their subscribed blockchain network 017c; and
Business object 015 is mapped to identity of client (brand or retailer) 174 and their subscribed blockchain network shard.

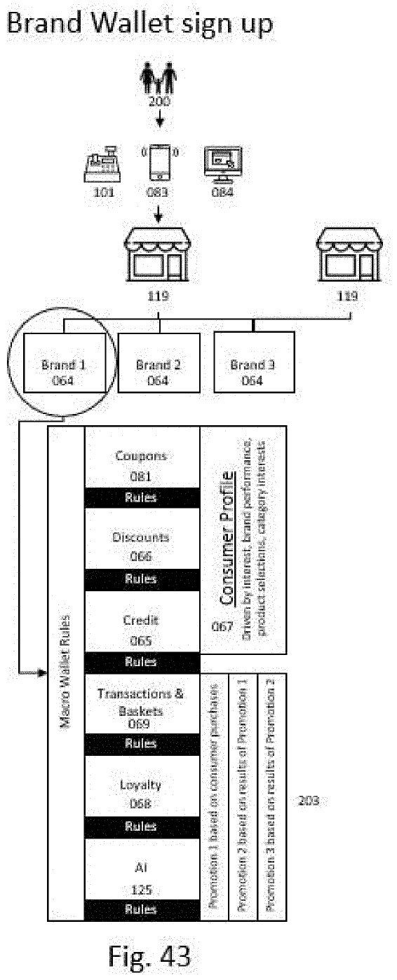

FIG. 43 illustrates a method for a consumer 200 signing up to a brand wallet 064 that allows a consumer 200 to access coupons 081, discounts 066, and brand credit 065 across multiple stores 119. The consumer 200 signs up for the program 064 at a POS 101, mobile device 083, online environment 084, or other through either the retailer 119 or brand 107 directly. This gives the consumer access to the brand wallet 064 at any subscribing retailer 119. The brand wallet 064 consists of a consumer profile 067 and a recommendation 023 engine. The consumer profile 067 hold all coupons 081, discounts 066, and brand credit 065 available or clipped for that consumer 200. Brand credit 065 is credit specific to the brand 107 and available to be spent across multiple retailers 119 or with the brand 107 directly. The consumer profile 067 also houses all consumer information, such as interest, historic performance, products selected, category interest, and other consumer indicators that may predict performance or preference. The recommendation 203 engine leverages this consumer information 067, historic and current transaction 069, and brand loyalty 068 to recommend new coupons 081, apply a discount 066, or grant brand credit 065 based on the rules dictated in the AI engine 125.

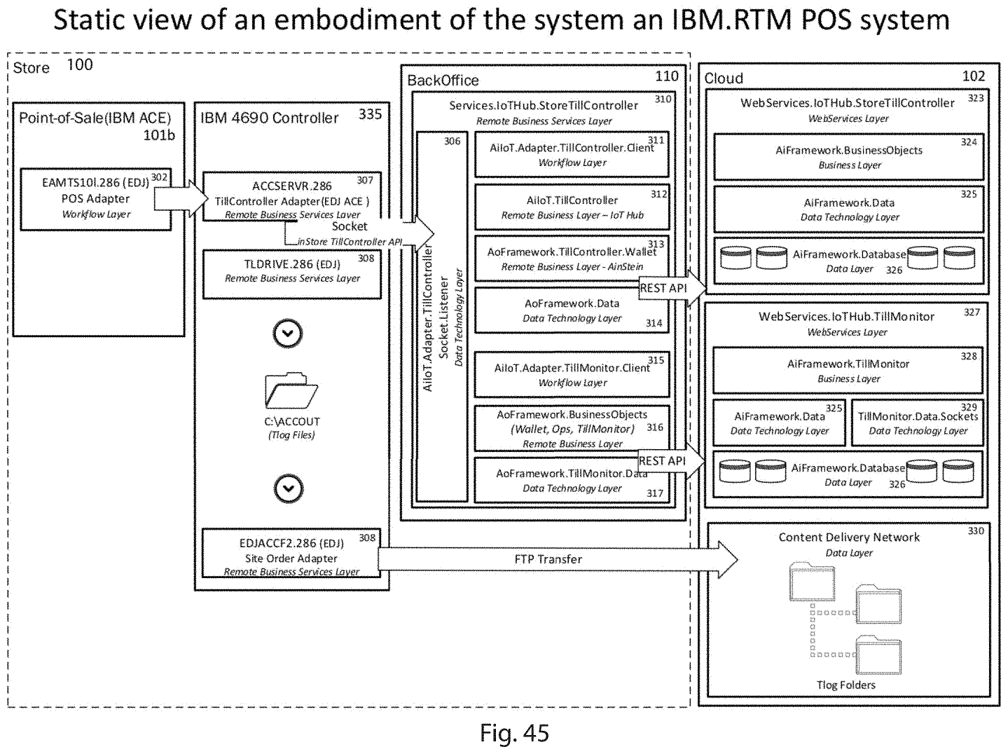

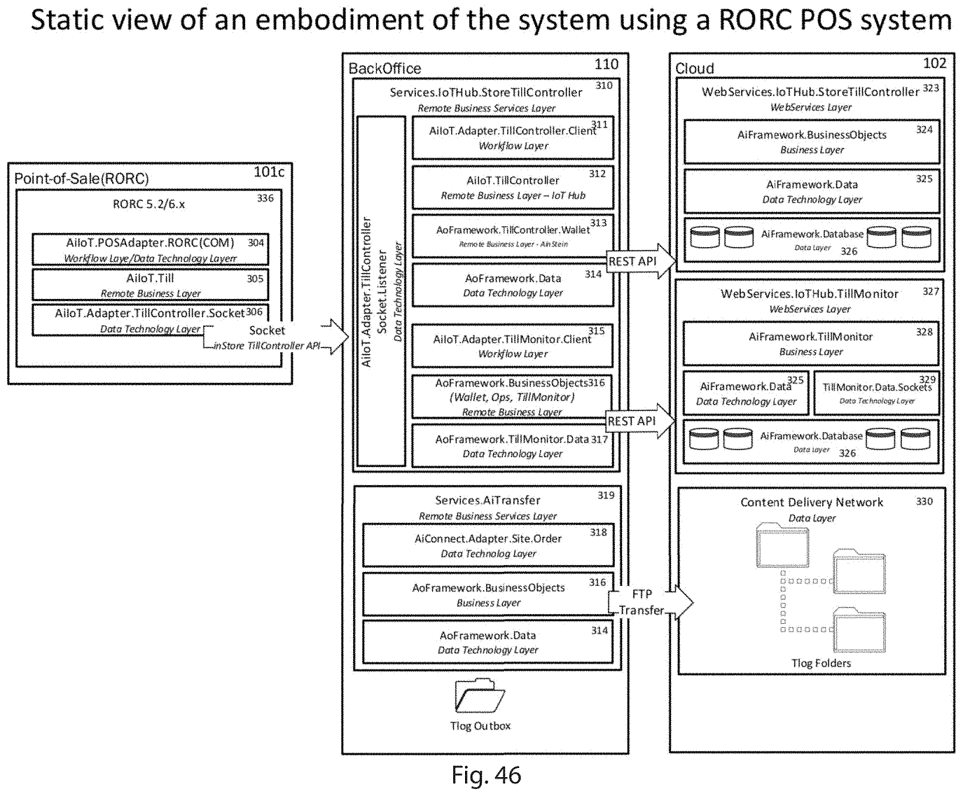

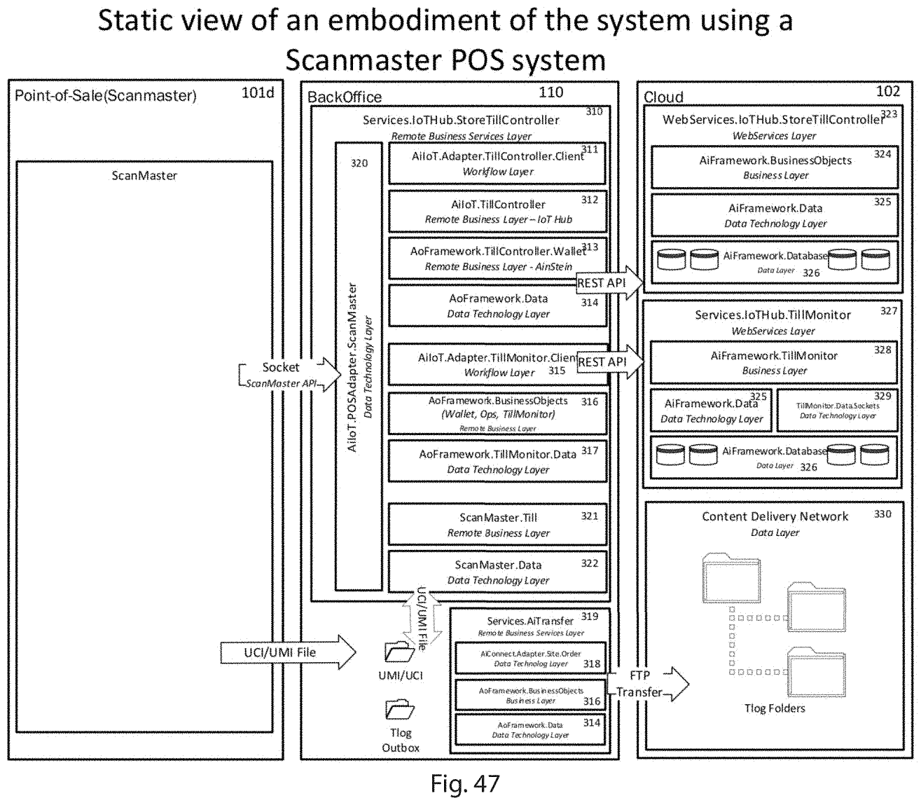

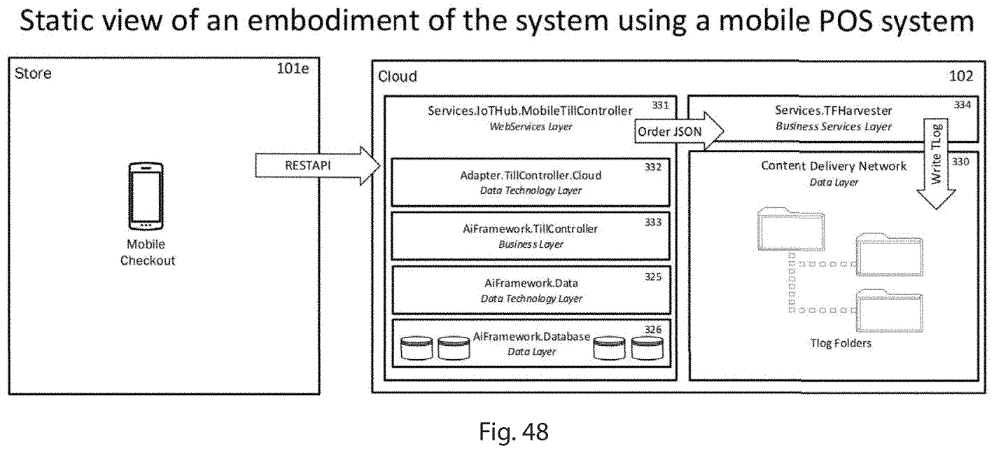

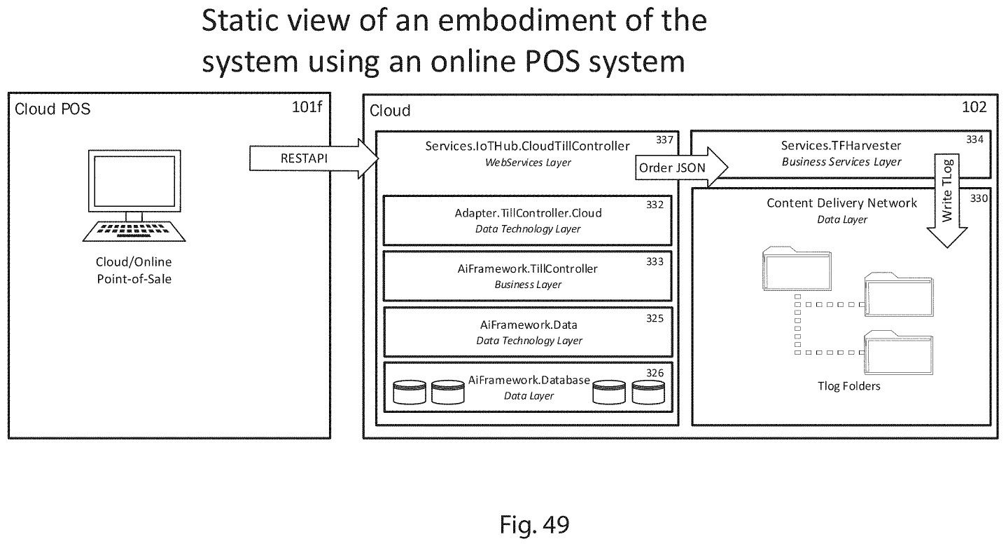

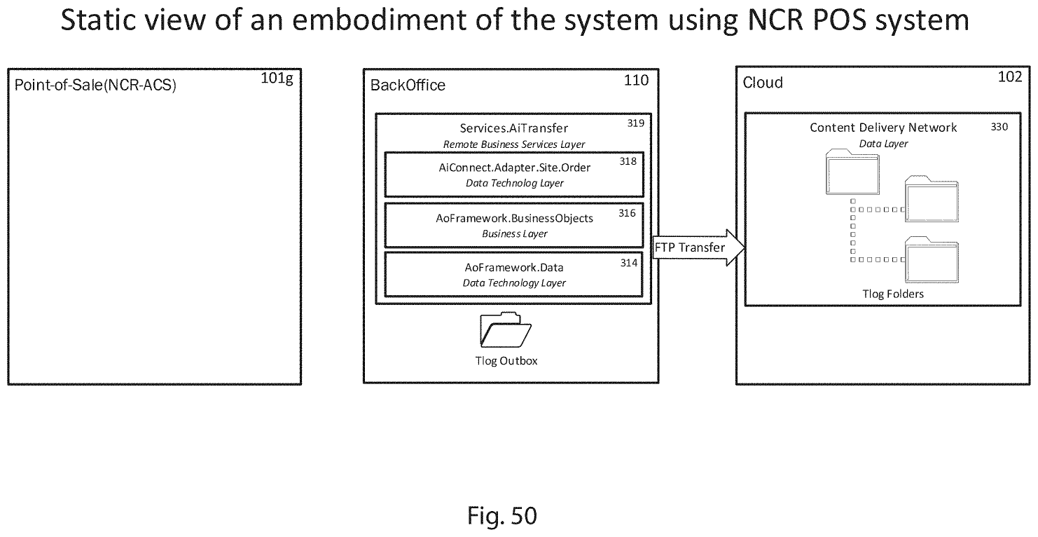

FIGS. 44, 45, 46, 47, 48, 49, and 50 illustrate static views of example embodiments of system comprising different types of POS systems and scales. FIG. 44 represents a static system framework diagram for use with a StoreLine POS 101a system from NCR Corporation. FIG. 45 represents a static system framework diagram for use with an IBM POS 101b system. IBM POS 101b is a retail checkout machine developed by International Business Machines Corporation. FIG. 46 represents a static system framework diagram for use with a RORC (Retail Owned Research Company) POS 101c system. RORC POS 101c is a retail checkout machine created by Dumac Business Systems, Inc. FIG. 47 represents a static system framework diagram for use with a ScanMaster POS 101d system from NCR Corporation. FIG. 48 represents a static system framework diagram for use with a Mobile POS 101e system. The Mobile POS 101e is a retail checkout that leverages a mobile phone where benefits to the order are determined by utilizing service-oriented architecture RESTful Web API communication. FIG. 49 represents a static system framework diagram for use with a cloud/online POS 101f system. The online or cloud POS 101f is a third-party cloud-based POS system that leverages an internet browser. Benefits to the online order are determined by utilizing service-oriented architecture RESTful Web API communication defined by Services.IoTHub.CloudTill controller 337. FIG. 50 represents a static system framework diagram for use with an NCR POS system from NCR Corporation. In these figures:

WPHook presentation layer 300 is a Retalix/NCR user exit module component of an ISS45 POS system that captures events and customizes behavior on ISS45 or StoreLine POS systems. ISS45 POS 101a is a retail checkout machine developed by NCR. The user exit module captures events and data within a POS checkout experience customized with benefits. The Wphook code may be written in legacy C code. The WPHook 300 acts as the user interface and allows the ISS45 POS 101a to communicate with the Services.IoTHub.StoreTill controller 310 in back office 110 through the AiIoT.Adapter.Till.Controller.Socket 306.

AiIoT.Adapter.ISS45.TillUI presentation layer 301 is an ISS45 IoT data adapter that translates events and data to a meet the standard POS adapter interface. This adapter handles special user interface interactions with the cashier displays. It turns legacy POS into an IoT device and ensures ISS45 C formatted event data is translated into standard format. This may be written using COM Interop C++ concepts that bridge the legacy WpHook C code to the C # library and .NET. This adapter sits within the UI WpHook 300. Events and data are translated to meet the interface requirements of AiIoT.POSAdapter.ISS45 303.

AiIoT.POSAdapter.ISS45 303 is a C # POS adapter for an ISS45 POS system 101a and is the entry point for bridging the legacy C/C++ technology of the POS to newer C # and .NET framework technology. This adapter allows the POS to become an IoT device. It handles all ISS45 events and data within C # and manages the communication to and from the POS system and to and from the Services.IoTHub.StoreTill controller 310. It sits within the WpHook 300 and utilizes the remote business layer AiIot.Till 305 and data technology layer AiIot.Adapter.Till controller. Sockets to communicate to and from the Services.IoTHub.StoreTill controller 310.

AiIoT.POSAdapter.RORC Data Technology Layer 304 is a C # POS adapter for RORC POS systems 101c and has a standard interface to deal with the standard events and data that support and enhance communication to and from the POS system and to and from the Services.IoTHub.StoreTill controller 310. This interface is derived from a standard interface used by all POS integrations and it gives a standard interface that describes the necessary events and data required to turn a legacy POS system into an IoT device by allowing for communication to the back-office server 110 through the AiIoT.Adapter.Till.Controler. Socket 306.

AiIoT.Till Remote Business Layer 305 is a lightweight set of C # business objects that hold minimal information and rules on context of the checkout activity. These objects cache necessary transaction data and perform lightweight business rules to optimize the POS system functionality. They provide the logic to support the presentation layer to and communicate to the back-office server 110 through the AiIoT.Adapter.Till.Controler. Socket component 306.

AiIoT.Adapter.Till controller. Socket Data Technology Layer 306 is a C # adapter used by some POS integrations that handle the socket communication via the instore till controller API 310 that communicates to and from the Services.IoTHub.StoreTillController 310. This adapter is used by each POS system within the store to communicate to the Services.IoTHub.StoreTillController 310 on a back-office server 110. The Services.IoTHub.StoreTillController 310 communicates back to each POS with benefits to be added to orders.

EAMTS10I.286 302 is a presentation layer adapter that lives within an IBM ACE 101b POS system. This adapter manages special user interface interactions with the cashier and communicates with the ACCSERVR.286 307. This is written in C to work with the IBM 4690 335 technology. IBM 4690 Controller 335 is the operating system that manages the IBM POS 101b.

ACCSERVR.286 Remote Business Services Layer 307 is the IBM 4690 controller C code written to capture POS events and data that support and enhance communication to and from the POS system and to and from the Store Till controller. It is the IBM equivalent to the AiIoT.Adapter.Till controller.Socket component 306. Code is written in C to work with IBM 4690 technology. This code allows communication between the IBM POS 101b and the Services.IoTHub.StoreTillController 310. This code resides on the IBM 4690 controller 335.

TLDrive.286 Remote Business Services Layer 308 is the IBM 4690 controller C code written to work on the IBM back-office controller 335 to create TLog files and place them in a folder for transfer to the CDN 330. Code is written in C to work with IBM 4690 335 technology.

EDJACCF2.286 Remote Business Services Layer 309 This IBM 4690 controller C code that transfers ACE and SA TLog files to an FTP location within the CDN 330. The code resides on the IBM 4690 335 controller.

Services.IoTHub.StoreTillController (instore till controller API) 310 remote business services layer is the agent on store back office server that communicates with the POS lanes and with the cloud 102. It serves as the remote IoT Hub to manage all legacy POS devices 101. It processes real-time scans at the register to determine rewards (e.g., coupons, points, and third-party rewards) to be added to the order, communicates back to the cloud 102 on rewards settled, and adds rewards for both loyalty and non-loyalty customers. This is the benefit engine that manages all POS systems 101 in the store and the order activity occurring at checkout. It communicates with the cloud 102 for customer wallet information and returns to the POS systems 101 wallet information and benefits to be added to the order. The inStore till controller API is used by the AiIoT.Adapter.Till controller. Socket 306 to communicate from the POS to the Services.IoTHub.StoreTillController 310. This API can also be used by third-party loyalty systems to integrate functionality. This business service is supported by underlying components and packages AiIoT.Adapter.Till controller.Client 311, AiIoT.Till controller 312, AoFramework.Till controller.Wallet 313, AoFramework.Data 314 to manage the POS IoT Hub and Ainstein processing logic.

AiIoT.Adapter.Till controller.Client 311 is a remote store (client) adapter used by the Services.IoTHub.StoreTillController 310 that supports a standard interface to interact with AiIoT.Till controller logic 312 within a Remote Business Tier. This provides the entry point for access to both POS IoT Hub and AinStein logic. All till controller adapters inherit from a standard till controller interface that allows all calls to the instore till controller API 310 to be either handled at a remote store back office machine or forwarded to other locations such as the cloud.

AiIoT.Till controller 312 is a set of business objects that manage the digital twins of POS systems within a store. This defines POS IoT Hublogic. The POS-IoT Hub functionality is structured to handled one to many stores and their POS systems. This logic is then used within the cloud to support unlimited number of stores or virtual/cloud POS systems. It is a robust set of logic to handle moving the IoT Hub to different tiers in the architecture.

AoFramework.Till controller.Wallet 313 is a set of remote business object components that provide remote caching and the AinStein business rules that determine order benefits. These business objects parse POS device orders and determine what benefits should be applied to an order and to a customer for later use. This component is the heart of the benefit logic. This is more commonly called AinStein and is complex logic that contains the intelligence to support all promotions.

AoFramework.Data 314 is the data technology layer that supports the OWM. This logic will take a remote business objects and map methods and data (in the form of JSON) to its equivalent webservice counterpart. This data technology component supports all remote business objects. This keeps all web service-related communication code out of each remote business objects. This component has abstracted away the common operations and data required to support standard fine and course grained calls to a webservice. This represents the OWM logic.

AiIoT.Adapter.TillMonitor.Client 315 is a remote store (client) adapter that provides a standard interface for till controller 310 events and data. It attaches itself to the POS IoT Hub where it transmits all till controller 310 operations and data over a websocket to a subscribing cloud service. This adapter allows events and data associated with activity at each POS Device 101 to be transmitted to a cloud 102 using the RESTful Web API WebServices.IoTHub.TillMonitor 327 where it can be used for additional services such as real-time analytics, AI, reporting and monitoring.

AoFramework.BusinessObjects [Wallet, Monitor, Operations] 316 are remote business components that contain caching and light business rules used to validate data submitted to Web Services.IoTHub.TillMonitor 327. The business objects are used to allow remote systems to be developed in the same fashion as cloud-based services 327. This gives consistency and structure to all development regardless of what physical tier business objects will reside on. This allows services to be moved across tiers with limited intervention.

AoFramework.TillMonitor.Data 317 is a data technology component that uses websockets to transmit real-time POS IoT Hub activity to the cloud 102 using the RESTful WebServices.IoTHub.TillMonitor 327. This component is a special data layer that provides and accepts events and data that are received over a websocket from and to subscribing monitor cloud services 327 through the TillMonitor.Data. Sockets 329.

AiConnect.Adapter.Site.Order 318 is an adapter used by the TLog Transfer service (AiTransfer) 319 to properly locate and batch send orders to the system CDN 330.

Services.AiTransfer 319 is a remote business service responsible for batch transferring TLog orders created from IoT POS Device 101 activity. In addition, it has an option to extract store product files from back-office server 110 database system and send to the system CDN 330. AiTransfer 319 knows where to transfer files using the space definition based on its physical location. Once AiTransfer 319 is authenticated it adjusts that name of files as well as where the files will be written to. Files are transferred using secure ftp.

AiIoT.POSAdapter.ScanMaster 320 Integration to the ScanMaster POS system differs from other POS systems as the POS Adapter resides on the back-office server 110 rather than on the POS 101d. This adapter handles communication to and from each POS system using UCl/UMI files, which are ScanMaster file formats. UCI is for communication from the POS to a third-party integrator, and UMI is for responses from the third-party integrator to the POS. This is the communication protocol that allows third parties to integrate with a ScanMaster POS. This adapter is supported by the ScanMaster.Till 321 business layer and ScanMaster.Data 322 data technology layer.

ScanMaster.Till 321 is a set of business objects that provide light business rules and caching to support the UCl/UMI method of communication with each POS device 101d.

ScanMaster.Data 322 are components support the asynchronous reading a writing of UCI and UMI files. Files are read to determine events occurring on the POS 101d and files are written to communicate back information and benefits to be placed within an order.

Web Services.IoTHub.StoreTill controller 323 is a RESTful API Webservice that supports the store till controller POS IoT Hub from the cloud 102. Provides operations and data that support all POS device customer interactions as well as back office server 110 functionality.

AiFramework.BusinessObjects 324 are a set of business components that represent the mental model of the problem space. These objects encapsulate all relationships, business rules, and caching intelligence. In addition, they manage and encapsulate all transactions to and from a relational database 017a. These business components make up the heart of the system and represent the collective intelligence of the system.

AiFramework.Data 325 is a set of base data objects that all business objects inherit from. They contain all the ORM logic that manage all relational database 017a interactions. This also contains sharding intelligence that allow space definitions to seamlessly determine what database each business objects will interact with. This package contains the logic rules on ORM. It abstracts away all relational database interactions from the business objects. It gives a common structure, naming convention, transaction management to all business objects that interact with relational databases.

AiFramework.Database 326 is the set of enterprise and sharded databases that make up the system and represents the data tier of the architecture.

WebServices.IoTHub.TillMonitor 327 is a RESTful WebAPI that supports the store till controller POS IoT Hub monitoring service 315. It handles all POS events and data and provides this information to subscribing presentation layers.

AiFramework.TillMonitor 328 is a set of business objects that support the TillMonitor service 327 providing light business rules and caching of store till controller 310 monitoring activity.

TillMonitor.Data.Sockets 329 is a component that handles the submission and acceptance of store till controller monitoring 315 events and data. This component links itself to any store till controller 310 as well as till monitor application for presentation of real-time events and control of a store till controller 310.

CDN 330 is a set of content servers used to store real-time TLog order and product files. This network of servers handles high throughput ftp transfers as well as processing performed by TLog harvester and product harvester services within the Services.AiTransfer 319.

Services.IoTHub.MobileTill controller 331 is a webservice that supports mobile checkout systems 101e. It defines and manages APIs, such as the instore till controller API but within the cloud 102. It has both POS-IoT hub and Aistein logic to support more modern and cloud POS systems 101e. This webservice is supported by underlying component and packages Adapter.Till controller.Cloud 332, AiFramework.Till controller 333, AiFramework.Data 325, and AiFramework.Database 326.

The Services.IoTHub.CloudTill controller 337 is a RESTful webservice that supports cloud checkout systems. It defines and manages APIs such as the instore till controller API 310 but within the cloud. It has both POS-IoT Hub and Aistein logic to support more modern and cloud POS systems. The Services.IoTHub.CloudTill controller 337 is supported by underlying component packages Adapter. Till controller. Cloud 332, AiFramework.Till controller 333, AiFramework.Data 325, and AiFramework.Databases 326.

Adapter.Till controller.Cloud 332 is a data technology layer adapter that supports the standard till controller interface. This interface is equal to the Adapter.Till controller.Client 331 interface but is designed to work within the cloud 102 rather than a remote store location 100. By using a standard till controller interface allows the POS IoT Hub and AinStein logic to move to different tiers within the architecture to optimize both performance, flexibility and product updates. It is used by the Services.IoTHub.MobileTill controller 331 and Services.IoTHub.CloudTill controller 337 webservices for cloud and mobile based POS systems.

AiFramework.Till controller 333 is identical code to the AoFramework.Till controller.Wallet 313 but runs in the cloud 102 rather than the remote client 100. It is the set of business objects that support the powerful AinStein logic that process orders and determines customer benefits. It is used by the Services.IoTHub.MobileTill controller 331 and Services.IoTHub.CloudTill controller 337 webservices for cloud and mobile based POS systems.

Services.TFHarvester 334 is a series of services to properly process POS specific transaction log files between the cloud till controller 337 and the CDN 330. Main processing engine to parse different TLog files formats into a normalized format. Loads appropriate TLog adapter to properly parse files.

RORC 5.2/6.x336 is the RORC POS system software 101c.

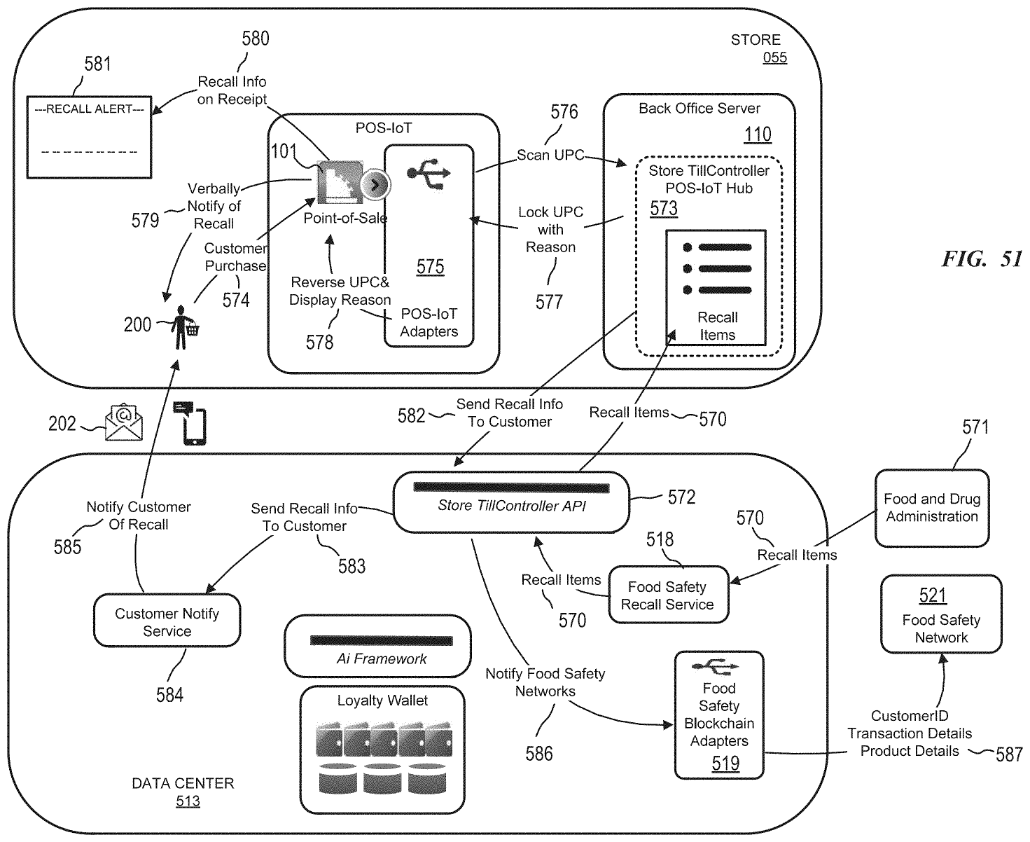

FIG. 51 depicts a process for locking a UPC to protect consumers from purchasing a product currently in recall from a POS device. A recall notice 571 is generated by the FDA, CDC, a brand 107 or other public or private agency 571 and sent to a food safety recall service 518 in system datacenter 513. Food safety recall service 518 imports the recall details and makes them available to store till controller API 572. A store till controller POS-IoT Hub component 573 in back office server 110 retrieves the recall details from store till controller API 572 and caches the recall items locally.

Customer 200 makes a purchase 574 from POS device 101 in store 055. POS 101 uses adaptors 575 to communicate with back office server 110. POS-IoT adapter receives a UPC scan from POS 101 and sends it 576 to store till controller 573 for recall validation. If the product to be purchased was included in a recall item notice 570, then store till controller 573 will return 577 a lock UPC response along with a reason that the UPC is being locked. This lock will prevent purchases of a recalled product at POS 101. POS-IoT adapter 575 then reverses 578 the purchase within POS 101 and displays the reason for the canceled or blocked transaction to the cashier at POS 101.

When the cashier receives a message on POS 101 that the transaction for the recalled product has been reversed, the cashier can then verbally 579 notify the customer 200 of the reason for the reversal. Additionally, or alternatively, the recall information associated with the locked UPC may be written 580 on a transaction receipt 580 to provide customer 200 with additional details. An example recall alert on a receipt would be: “Your purchase of: [product] has been recalled. Please return the product to the store and visit FSIS.USDA.GOV for more information.”

In addition to notifying customer 200 at POS 101 at the time of a transaction or an attempted transaction, back office server 110 may also send recall information to customer 200. Store Till controller 573 sends 582 a message to the store till controller API 572 to notify customer 200 by another method communication of the UPC lock activity at POS 101. Store Till controller API 572 then notifies 583 a customer notify service 584 of the recall notification action. Customer notify service 584 then sends 585 a notification 202 to customer 200 alerting him or her to the recall. The content of notification 202 may include details and/or a link to a website with more information about the recall. The notification 202 may be a telephone call, text message, email, application notification, postal mail, or any other method of communication.

The attempt to purchase a recalled product may also trigger a notice to food safety networks. Store till controller API 572 sends a notice 586 to food safety network 521. The notice 586 may be sent through a required blockchain adapter 519. The notice 587 to food safety network 521 from blockchain adapter 519 may include a customer identifier, transaction details, product details, or any other critical activity details written to each blockchain network.

FIG. 52 depicts an embodiment of a digital wallet 085 application that interacts with the system 130 and is incorporated within phones, tablets, computers, websites and other electronic devices. The digital wallet 085 allows the consumer to select pay, promotions, or rewards 086. The selection of the payment method will allow the consumer pay via digital debit 089 or digital credit 088. The digital wallet 085 will allow payments in any tender or combinations of tender. A consumer 200 may pay with a combination of tenders (e.g., fiat currencies, digital currencies, crypto currencies, security tokens, traditional credit, digital credit, digital debit, coupons, reward points, precious metals (tokenized), etc.). The digital wallet application will then allow the consumer to choose the tender method, fiat currency, utility tokens, and/or security tokens as well as to apply incentives and/or rewards. The digital wallet 085 may also allow the use of traditional debit and credit cards. Further, the digital wallet application may enable consumers to manage and access promotions and rewards across the consumer retail ecosystem.

FIG. 53 depicts an embodiment of the asset-side 090 of a balance sheet for a consumer credit lender that holds digital credit receivables on balance sheet and recorded on the blockchain. FIG. 53 also illustrates the liability and equity side 091a of the balance sheet for a consumer credit lender that has credit, time, or used other methods to tranche, represent, and/or fund risk via security tokens. Digital credit receivables may be held on balance sheet and funded by traditional methods or via the issuance of security tokens as shown in FIG. 53. Consumer digital credit receivables held on the asset side of the balance sheet 090 and represented, managed, and serviced on the blockchain 071c or other database method. Consumer digital credit receivables are sold (contributed) to a bankruptcy remote special purpose entity and represent collateral for the issuance of security tokens from which the proceeds fund the purchase. Consumer digital credit receivables are pooled into pass-through, time, credit, and/or other tranche methods with collateral performance recorded on the blockchain (or other database method) and ownership represented as security tokens (or other tokens) which is also recorded on the blockchain 017c or other database method. Security tokens (or other tokens) are held by the original lender and sold to other lenders to finance the portfolio of receivables. Security tokens (or other tokens) may be listed and traded on electronic exchanges.

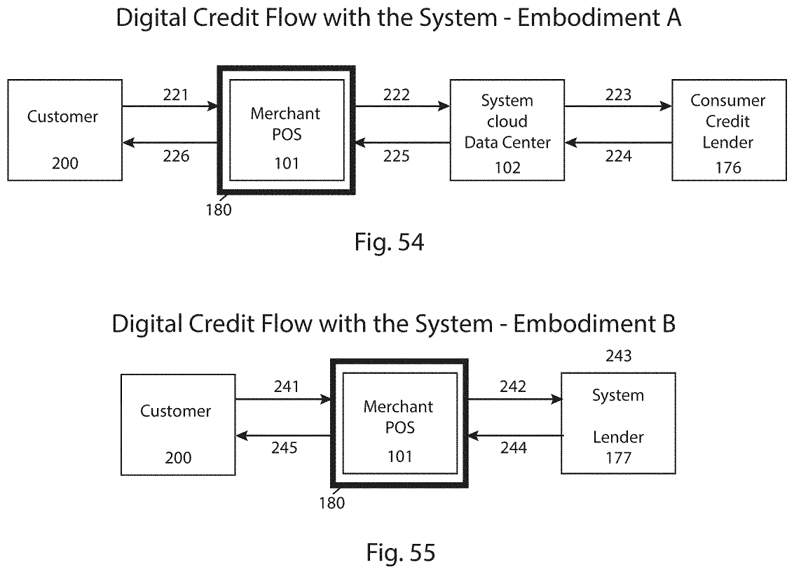

FIG. 54 illustrates an embodiment of the digital credit process flow within a retail eco-system. The merchant POS 101 may be brought into the system 130 by the system framework 180. Credit information of a consumer 200 may be entered into the merchant's payment system, the POS terminal 101 or an e-commerce website or other manner. A digital representation of the POS 152 maybe assigned to the merchant POS 101. A consumer chooses digital credit for payment on their digital wallet app 085 or API 126 to pay for a product or service 221 at the POS 101. Credit information may be sent to the merchant's bank, which then routes the data 062 through the payments system for processing or the data may be routed by the system 130. Merchant's bank may send the data to the system cloud data center 102 or affiliate company, which forwards it to the consumer's credit lender 176. Consumer's credit lender 176 verifies the digital credit is valid and the account has available credit to pay for the transaction 223. Consumer's credit lender 176 generates an authorization number and routes this number back to the system cloud data center 102 or affiliate company System cloud data center 102 (or affiliate company) may forward the authorization code back to the merchant's bank/merchant 225. Merchant (merchant POS 101) concludes the sale 226 with the customer 200. A direct payment (e.g., direct account-to-account, ACH payments, digital wallet to digital wallet, etc.) from the consumer's credit lender to merchant's account (or digital wallet) may then be executed and recorded on the blockchain (or other database method) to pay the retailer for the product 118 or services. Simultaneously, a digital credit receivable (and payable) between the consumer's credit lender 176 to the consumer 200 may be executed and recorded on the blockchain 017c (or other database method) to record the debt obligation to be satisfied for the consumer's credit lender payment of the product 118 or service, on behalf of the consumer 200.

FIG. 55 illustrates an embodiment of a digital credit process flow within the retail eco-system. The merchant point or sale 101 is brought into the system 130 by the system framework 180. Credit information of a consumer 200 may be entered into the merchant's payment system, the POS terminal 101 or an e-commerce website. A digital representation of the POS 152 may be assigned to the merchant POS 101. A consumer 200 may choose digital credit for payment on their digital wallet app 085 or API 126 to pay for a product or service 241 at the POS 101. Credit information may be acquired by system and recorded on a blockchain or other secure repository 017c. Credit information may be entered into the merchant's payment system, the POS terminal 101 or an e-commerce website, a blockchain record, credit reporting agency, etc. Credit information may be sent to the merchant's bank, which then routes the data through the payments system for processing (or banks may be completely eliminated in the process). Merchant's bank may send the data 062 back the system 130 (or the bank may be eliminated completely). The system 130 may verify that the digital credit is valid, and the account has available credit to pay for the transaction 242 (or system affiliate company)2. The system 130 (or system lender 177) may then generate an authorization number 243 and routes this number back to the merchant's bank/Merchant POS, merchant system or merchant's digital wallet (if bank are eliminated). Merchant concludes the sale with the Customer 245. A direct payment (e.g. direct account to account, ACH payments, digital wallet to digital wallet, etc.)) from the system lender 177 to merchant's account is then executed and recorded on the blockchain 017c (or other database method) to pay the retailer for the product 118 or services. Simultaneously, a digital credit receivable (and payable) between the system lender 177 and the consumer 200 is executed and recorded on the blockchain 017c (or other database method) to record the debt obligation to be satisfied for the system lender payment of the product 118 or service, on behalf of the consumer 200.

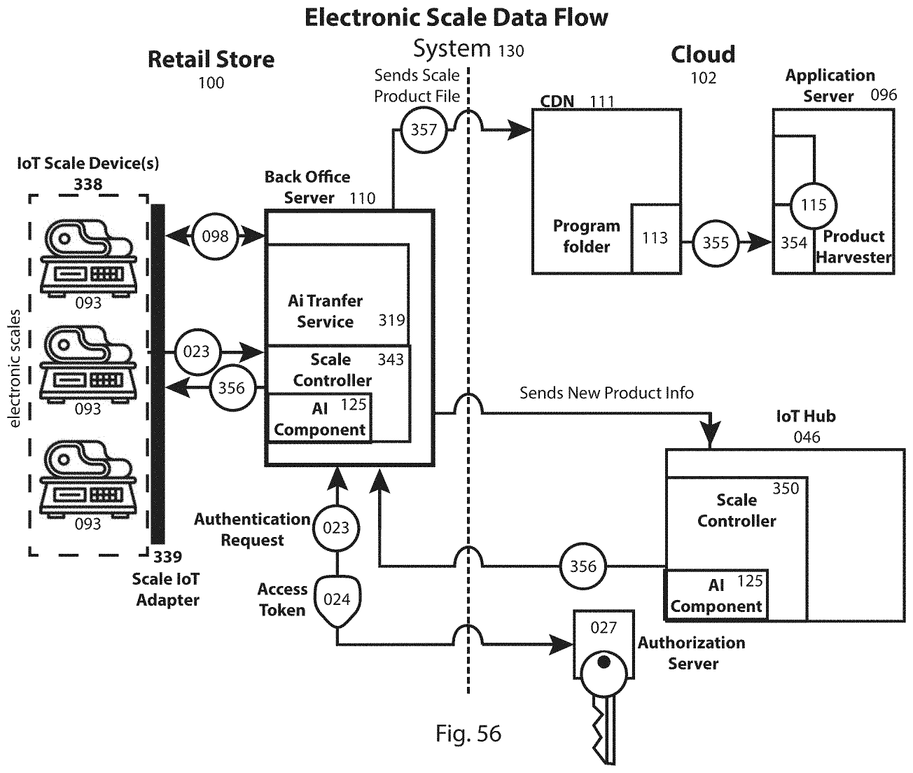

FIG. 56 depicts an embodiment of the system 130 showing electronic scale processing data flow. The communication process manages product transaction information from electronic scales 093, within retail stores 100 (both online and brick-and-mortar) to communicate with cloud 102 storage. When a store employee uses the electronic scale 093, the Scale IoT Device 338 communicates with the scale controller 343 through the scale IoT adapter 339. This authentication communication 023 is requesting OAuth 2.0 authorization (or other secure authorization standard) from the authorization server 027 in order to access the IoT Hub (Resource Server) 046. The IoT Hub 046 converts any legacy electronic scale device 093 into IoT scale device 338 by making a digital representation of the legacy scale device 093 that is IoT compatible. Once this authorization is confirmed an access token 024 is issued and the scale controller 343 requests any relevant data for the product 356. This product data 356 includes all providence and other product history relevant to the weighted item 358. The scale device 093 prior to transaction completion requests store and product information (e.g., new store identification number, food safety information, etc.) from the scale controller 343. The scale device 093 upon completing the transaction notifies the scale controller 343 and writes product information to the back office server 110. Another implementation of this is the scale controller 343 sends the completed product transaction to the cloud 102 scale controller 350. The back office server 110 may contain at least two separate binaries, The Ai Transfer Service 319 and the scale controller 343. Both of these services reside side by side on the back office server 110. Both the Ai Transfer service 319 and the scale controller service 343 must authenticate 023 with the authorization service 027 to receive an access token 024 that is used to allow access to the IoT Hub 046 (Resource Server). The scale controller 343 is made available to each electronic scale 093 via the scale controller API. Each scale type 093 has a scale IoT adapter 339 that is loaded by the scale device 093 to handle messages specific to the type of electronic scale 093. The adapter 339 accepts messages formed by the type of scale 093 and converts them to the standardized system format and sends this information to the scale controller 343 using the in scale controller API for processing. At completion of the request, the scale controller 343 settles order with the cloud 102 scale controller IoT Hub 046. At validation of completion the scale device 093 communicates 098 with the scale controller 343 for contents to be written to the receipt to be used for the weighted item which could include: Food safety details, product weight, time, or any other information that would be useful to the supply chain.

Back office server 110 stores the product data and batches communication to the CDN 111 using the Ai Transfer service 319 (or similar). This batch communication 357 mitigates server calls to manage the large amount of store product transactions. The CDN 111 organizes product files by program folders 113 to efficiently store this data 357. The data stored on the CDN 111 may be in native electronic scale language format 355. This product data is then harvested by the product harvester service 354. In harvesting the data, the native electronic scale language format 355 is converted into a standardized (normalized) data format 115 that is universal to the system.

The AI component 125 of the scale controller 350 takes the activity at the electronic scale 093 and determines the benefits to be added on the customer's behalf to the order. It should be noted that in order to support online ordering and mobile checkout the scale controller 350 is also within the IoT Hub 046. The AI component 125 can also be located in the cloud to allow us to support the migration to cloud 102 and mobile based POS systems 121. The scale controller 350 and the AI component 125 can to be placed in the cloud to service as the back office server 110.

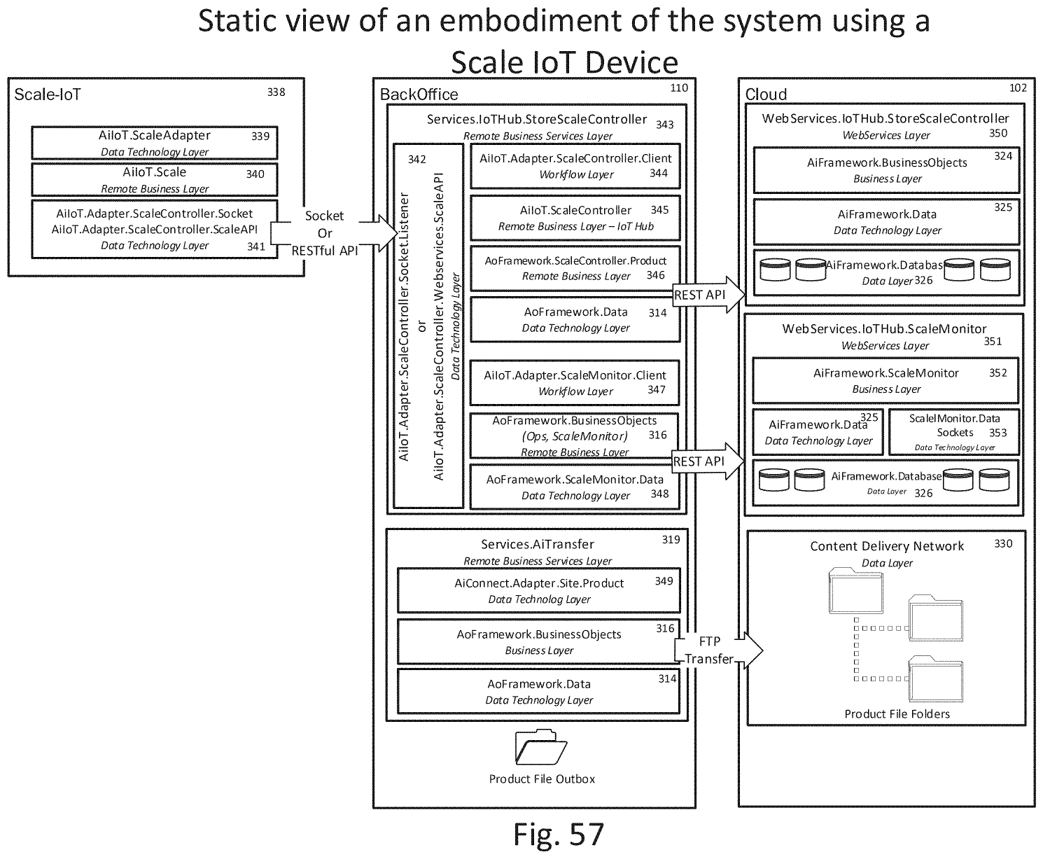

FIG. 57 depicts a static view of an embodiment of the system showing electronic scale processing data flow. The communication process manages product transaction information from electronic scales within retail stores (both online and brick-and-mortar) to communicate with the cloud 102. This diagram assumes the back-office server 110 store scale controller IoT-Hub 343 has successfully authenticated with the authorization server 027 and received an access token indicating authorization. Electronic scales are authenticated with store scale controller IoT-Hub 343 and a digital representation is created and managed within the IoT-Hub 343. Electronic scales that are used for processing weighted item product are converted to Scale-IoT devices 338 for the system 130 by using a scale adapter 339. This scale adapter 339 has business logic 340 that allows local processing to occur as needed before sending weighed item product information via either a socket adapter or ScaleAPI adapter 341 to the back-office server 110 store scale controller IoT Hub 343 or directly to the cloud Scale IoT Hub 350. This weighted item information includes product identification information, weight, price, and other as needed. The store scale controller IoT Hub 343 uses the client scale controller adapter 344 to support the common scale controller interface. The scale controller IoT Hub 343 data and business rules for each of the stores electronic scales are managed within the AiIoT.ScaleController 345 and business rules and data associated with the product used at each of the electronic scales are managed in AoFramework.ScaleController.Product 346. The AoFramework.Data 314 handles all data communications to the cloud 102 Webservices.loTHub.StoreTill controller 350.

The cloud 102 Webservices.IoTHub.StoreTill controller 350 manages digital representations of electronic scales through the AiFramework.BusinessObjects 324 and stores product information and other activity using the AiFramework.Data 325 module.

The store scale controller 343 can be monitored using the ScaleMonitor client adapter 347 it takes events sent to the store scale controller 343 from each of the store electronic scales and forwards them using the AoFramework.BusinessObject(SaleMonitor) 316 business layer and AoFramework.ScaleMonitor.Data that contains communication protocols for websockets and REST API. This data is forwarded real-time to the cloud 102 ScaleMonitor IoT-Hub 351. The AiFramework.ScaleMonitor 352 manages all data and rules around electronic scale events received and those messages that need to be sent to electronic scales. Product data storage is performed by AiFramework.Data 325 and real-time events are received and sent to the remote scale monitor client 347 using ScaleMonitor.Data.Sockets 353.

Weighted item product information and other activity that occurs on electronic scales is stored within data sources 326.

In another embodiment, the scale adapter 339 writes product weighted item file 357. The AiTransfer service 319 then periodically transfers these product files 357 to the content delivery network 330. These files are then processed by the product harvester 354 where product details are stored within a data source 326. The weighted item product information is associated with the appropriate store using space definitions around the Scale-IoT digital representation. This information can then be used with the transaction data 062a to determine the consumer associated with the weighted item product. This can be used to notify the consumer in case of food safety issues or related product issues. Other suitable scale-IoT types and configurations will be apparent to persons having skill in the relevant art.

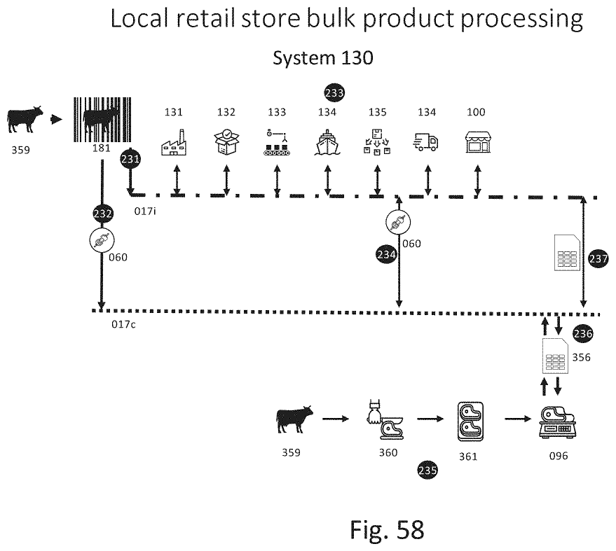

FIG. 58 illustrates tracking product processing at local retail stores within the system 130. This embodiment of the system 130 uses one (or more) third-party blockchains and/or databases. That include the following steps (not necessarily in order):

Creation 231—a brand specifies a UPC 181 for its bulk 359 product and registers the UPC 181 with a third-party blockchain network 017i.

Connect 232—the Brand registers the UPC 181 with the system's blockchain/databases 017c.

Product progression 233—during each step of the production and delivery process (i.e., production 131, packaging 132, shipping 133, transportation 134, distribution 135, other transport, delivery to retail 100), the product 359 is tracked via the third-party blockchain 017i.

The system's repository, blockchain, and/or database is approved with the third-party blockchain 017i to write 234 on behalf of brand. The system 130 writes to its own blockchain and/or database 017c and the third-party blockchain 017i on behalf of the brand. Using data processing adapters (e.g., block chain adapters and/or database adaptors) 060. The system's adapter technology 060 is used to write to brand registered third-party blockchain networks 017i.

In flow 235, the store processes 360 the bulk product 359 into smaller consumer products 361 and leverages an electronic scale 096 to weigh the product.

Product data 356 is written 236 to the system's blockchain network and/or databases 017c utilizing electronic scale 096 information 356.

Product information is also written 237 to the third-party blockchain network 017i via the system's block chain adapters 060.

In other embodiments, all processes may take place on the system's blockchain 017c or other secure database without the third-party blockchain 017i.

Product 359 data can be tracked (i.e., written to a repository) directly from the electronic scale 096 data or the product 359 data can be tracked (written to a repository) through the POS device 101 after being weighed and label by the electronic scale 096.

The system 130 may write this product information to various blockchain 017c and database 017a systems. Consumer purchase 108 of the product 359 via the retailer POS is also written to one or more repositories 017c and/or databases 017a, etc. Approved wholesalers, distributors and retailers 119 can enter the food origin information into the system thus providing greater traceability for individual products handled and portioned by them. The system 130 greatly enhances traceability and food safety for locally processed and portioned products.

FIG. 59 illustrates sensor and location data being received into the system 130. Location and other sensors 198 of all kinds are sending data via satellite 137 or other channels to the system 130. These sensors may be located at a brand 107, processing locations, manufacturing locations, in transportation 134 (e.g., ships, vans, trucks, drones, robots, etc.), inside packaging 138, or attached to the product 118. The sensors generate data 062. The system receives the data 062 via radio signals, WiFi, Bluetooth, or other communication method. The data my travel via satellite 137 or other manner. The system 130 may normalize the data 062 into a standard system language format and store the data 062 in a repository 017, database 017a, blockchain repository 017c, or other repository 017. The information is then available to be queried by the query module 141 to identify data 062 and criteria. Stored data 062 may be fed into an AI engine 125 (or other system algorithms) that can identify important data 062 or combinations of data 062 and generate responses, create offers, diagnosis issues, recommend actions, generate incentives, create games, etc.

Sensors 198 of all kinds can used to ascertain the quality of shipping. For example, if a product is sensitive to temperature, thermal sensors and thermometers might be used to monitor a products temperature during shipping. Or if a product is sensitive to vibration, a vibration sensor, accelerometers, gyroscopes, may be used to monitory the vibration crash events, etc. of the product 108.

That information monitored can be relayed to the system 130 and stored in a repository. That repository could be queried to identify the shipping conditions of the product and purchase and/or other decisions (e.g., processing, pricing, product designation, whether to use it in other products, etc.) could be made with that information and/or suggested by AI 125.

System AI 125 may develop personalized pricing for consumers and deliver tailor made discounts based on location, date, time, age of customer, sex of customer, passed behavior, and other criteria. May offer an incentive a customer for a second location based on past purchases and location of a customer. This not only ensures consumers will see what is most relevant to them, but also ensures that brands aren't incentivizing consumers unnecessarily. Games (gamifcation of incentives or games to promote good will) can be suggested or personalized for consumers 200 or groups of consumers as well. A group of consumers may all opt in to compete against each other for a prize (incentive, reward). AI may create a game based on criteria of every member of the group or other criteria. The group could that be given tasks to complete. Those tasks could be sent to individual communication devices 085 or printed to a customer receipt 094.

System AI 125 could suggest responses, predict out comes (e.g., damaged products, etc.), diagnose, anticipate (e.g., ETAs, just in time deliveries, etc.), incentivize (e.g., personized incentives based on multiple criteria), recommend, create games, and more.

Individualized incentives are important for brands. For example, a soft drink company does not need to offer a discount to consumer that purchases its product 100% of the time, those coupons (incentives) should be used to acquire consumers of competitive brands. Instead, the soft drink company can reward that loyal consumers with discounts on other products from the same brand or related portfolios.

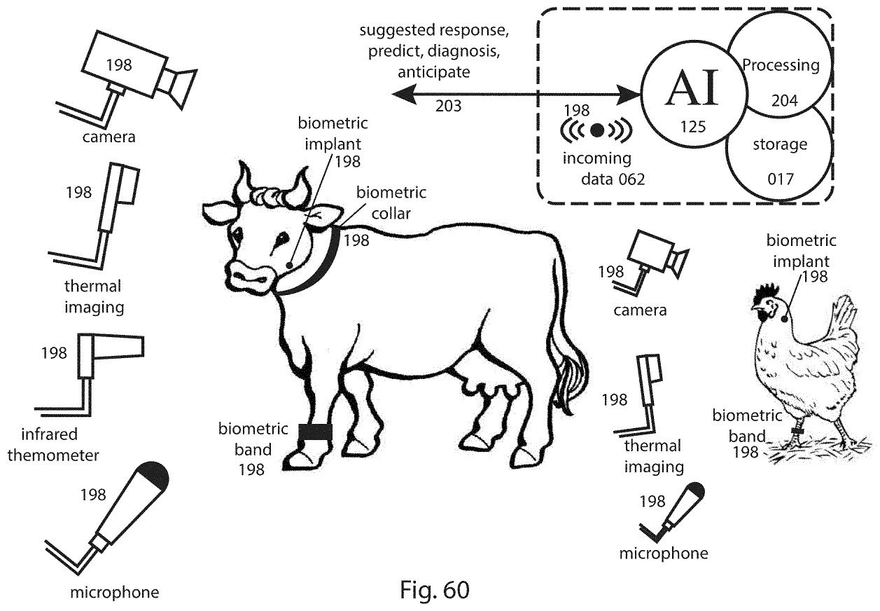

FIG. 60 depicts the use of AI 125 as part of the system 130 to help manage a safe food supply. The use of AI together with data from cameras and other sensors 198 help maintain animal health ensuring a better supply chain. Instead of relying solely on farmers' senses and knowledge, on-site sensors can provide reliable data about the physical condition of animals. AI can quickly analyze this data and provide diagnosis and suggestions on how best to care for the animals.

Cameras, sensors 198, and wearable technologies can be worn and/or implanted on animals to detect their sweat constituents, measure body temperature, observe behavior and movement, detect stress, detect pH, detect presence of viruses and pathogens, antibiotic detection. With data from these sensors, AI 125 can help analyze sound (e.g., coughs, animal noises), prevent diseases, diagnose disease early in order to prevent animal deaths. AI 125 can also recommend animal to be separated from the group in order to prevent the spread of disease. AI 125 can send recommendations, diagnoses, predictions, etc. to a caretaker's cell phone for quick response. All of this to help insure a better life for animals and better-quality food supply.

In areas with animals, cameras, infrared thermometers, thermal imagers, microphones, and other biometric devices and sensors 198, collect data and send it to be analyzed via radio signals, Bluetooth, WiFi, or other communication methods. The data is process stored and analyzed. AI 125 makes recommendations, predictions 203 for further action by an animal caregiver.

In a like manner, sensors 198, such as oxygen sensors and wearable technologies, can be worn and/or implanted on humans detect their sweat constituents, measure body temperature, observe behavior and movement, detect stress, detect pH, detect presence of viruses and pathogens, antibiotic detection, detect sounds, etc. AI 125 can send recommendations, diagnoses, predictions, etc. to an individual 200 or an individual's caretaker (e.g., via cell phone or other manner) for quick response. All of this to help ensure better quality of life for individuals.

A mobile device 083 may be programed to record sound data (e.g., microphone on a cell phone) AI 125 can help analyze human sounds, such as human coughs, sneezes, differences in voice from day to day, to help diagnose disease early, in order to prevent disease, catch health issues early in order to improve health and extend lives.

AI 125 may recommend actions such as doctor visits, supplements, rest, exercise, over the counter medications, change in diet, etc.

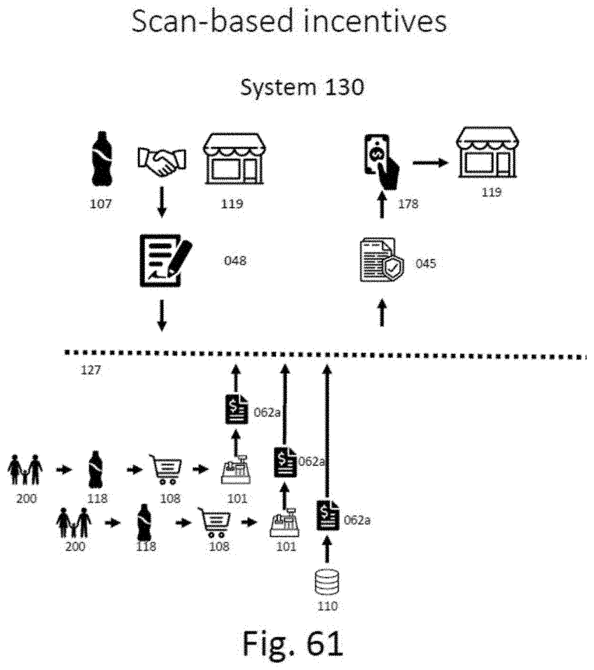

FIG. 61 illustrates the use of scan-based incentives within the system 130. Scan-based machine-to-machine incentives are a novel aspect of the system in which retailers and brands can track and exchange scan-based offers on a trusted blockchain network 127 all while being able to track sales in real time (or near real time). The system provides a method to settle scan-based incentives machine to machine based on sales information 062a written to the blockchain 127 from transaction logs processed at a retailer's POS 101. A scan-based incentive smart contract 048 is written and when certain criteria is reached (e.g., sales in a certain time frame, sales goals, etc.) the retailer automatically receives payment. Actual sales information 062a may be written to the blockchain 127 from the retailer POS data and the scan-based incentive smart contract 048 is settled automatically on a trigger event(s) 045 when certain criteria are met. By writing retailer product sales data 062a from a POS machine or machines at retailers' premises 119 to a ledger on a blockchain 017c, human intervention is eliminated thus enabling greater trust between the brand 107 and retailer 119. Further, this blockchain-based novel solution will mitigate or eliminate brand-retailer transaction errors and/or fraud and accelerate payments between the brand 107 and retailer 119.

A smart contract 048 is written to settle when certain criteria are reached, such as when a completed smart contact is triggered 045 the retailer 119 automatically receives payment 178. The smart contract 048 between a brand 107 and a retailer 119 is settled machine-to-machine based on sales information 062a written to the blockchain 127 from the retailer POS 101 or back office systems 110. Actual sales information 062a is written to the blockchain 127 from the retailer POS 101 or back office system 110 then the incentive is settled when certain sales criteria is reached (e.g., sales in a certain time frame, sales goals, etc.).

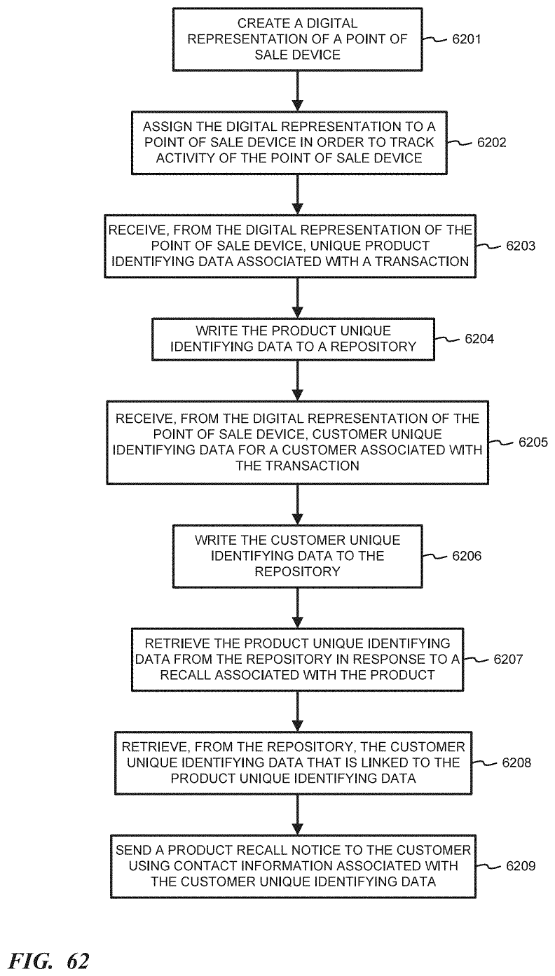

FIG. 62 is a flowchart illustrating a process for sending a product recall notice to a customer in an example embodiment. In step 6201, a digital representation of a point of sale device is created. In step 6202, the digital representation is assigned to a point of sale device in order to track activity of the point of sale device. In step 6203, unique product identifying data that is associated with a transaction is received from the digital representation of the point of sale device. In step 6204, the product unique identifying data is written to a repository. The repository may be a blockchain repository in some embodiments. In step 6205, customer unique identifying data for a customer that is associated with the transaction is received from the digital representation of the point of sale device. The customer unique identifying data may be associated with a customer payment instrument. In step 6206, the customer unique identifying data is written to the repository.

In step 6207, the product unique identifying data is retrieved from the repository in response to a recall associated with the product. In step 6208, the customer unique identifying data that is linked to the product unique identifying data is retrieved from the repository. In step 6209, a product recall notice is sent to the customer using contact information associated with the customer unique identifying data.

The process for sending a product recall notice to a customer may further include sending product recall information and unique identifying data of the customer who purchased the product to the payment processor associated with a customer's payment instrument transaction account. The transaction account may be a credit account. The product recall notice may be sent to the customer by a payment processor.

The process for sending a product recall notice to a customer may further include crediting the customer's payment instrument transaction account with the purchase price of a recalled product. The process for sending a product recall notice to a customer may further include translating data from a native point-of-sale language to a standard system language format.

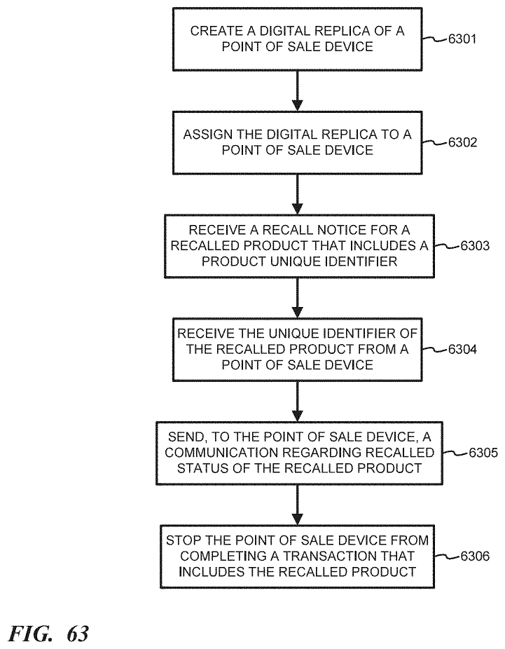

FIG. 63 is a flowchart illustrating a process for stopping a purchase of a product that has been recalled in an example embodiment. In step 6301, a digital replica of a point of sale device is created. In step 6302, the digital replica is assigned to a point of sale device. In step 6303, a recall notice is received for a recalled product. The recall notice includes a product unique identifier. In step 6304, the unique identifier of the recalled product is received from a point of sale device. In step 6305, a communication regarding recalled status of the recalled product is sent to the point of sale device. In step 6306, the point of sale device is stopped from completing a transaction that includes the recalled product.

The process for stopping a purchase of a product that has been recalled may further include tracking point of sale device activity via the digital replica of the point of sale device.

The process for stopping a purchase of a product that has been recalled may further include receiving, from the digital replica, the unique identifier of the recalled product. The process for stopping a purchase of a product that has been recalled may further include causing information regarding the recalled product to be printed on a receipt.

The process for stopping a purchase of a product that has been recalled may further include causing to be displayed at the point of sale a notice about the recalled status of the product.

The process for stopping a purchase of a product that has been recalled may further include translating data from a native point-of-sale language to a standard system language format.

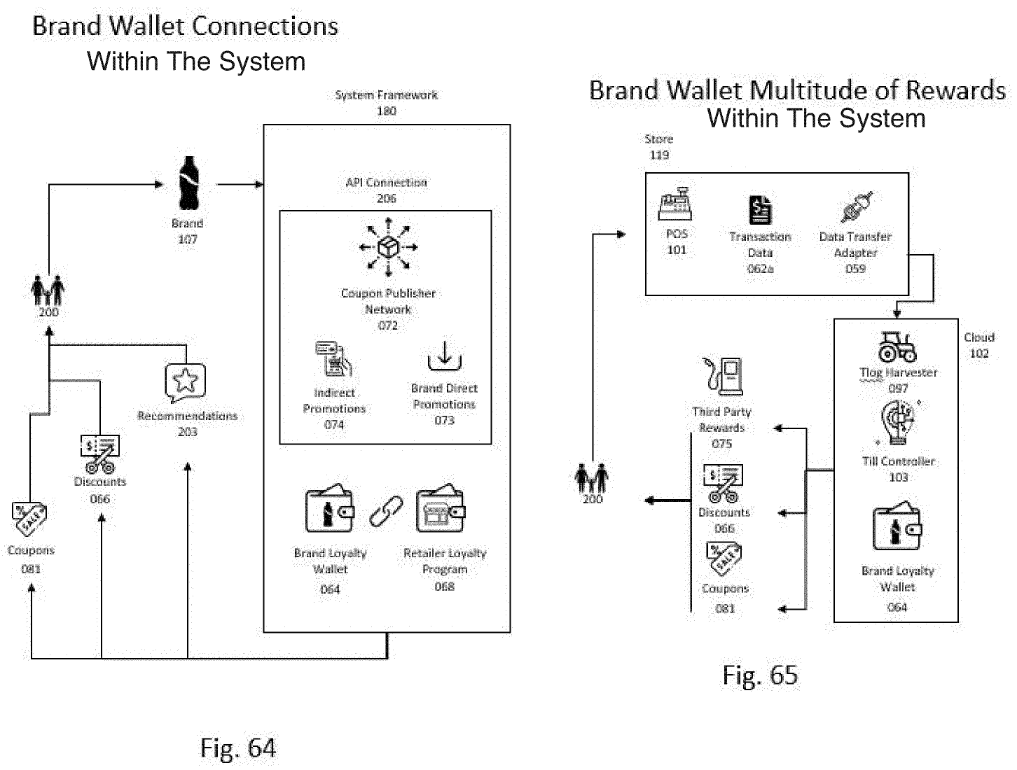

FIG. 64 illustrates a method of operation with a system framework 180 in which a consumer 200 receives coupons 081, discounts 066, and recommendations 203 from both a brand 107 and a retailer 119. The consumer 200 can sign up directly with the brand 107. This connect the consumer to benefits from the brand loyalty wallet 064 and the retailer loyalty program 068. Both the brand 064 and retailer program 068 are linked together. Coupon publisher networks 072, indirect promotions 074, and brand direct promotions 073 are injected into the system. Direct promotions 073 come from the brand 107, whereas indirection promotions 074 originate from third-party coupon providers. An AI 125 and wallet logic 064/068 provide the consumer with unique recommendations 203 from the promotion content sources 072, 073, 074.

FIG. 65 illustrates a method of operation in which a consumer 200 receives various benefits 081, 066, 075 after purchasing a product from a retailer 119. When the consumer 200 purchases the product at a store 119, the transaction data 062a is sent from the POS 101 to the cloud 102 through a data transfer adapter 059. This adapter 059 communicates with the TLog harvester 097. Till controller 103 filters this data into the appropriate brand loyalty wallet 064. Then, the wallet 064, though imbedded logic, triggers or pushes benefits to the consumer 200. These benefits include, but are not limited to, coupons 081, discounts 066, and third-party rewards 075. The third-party rewards include things such as airline miles, points for gas, free entertainment subscriptions, movie tickets, and others.

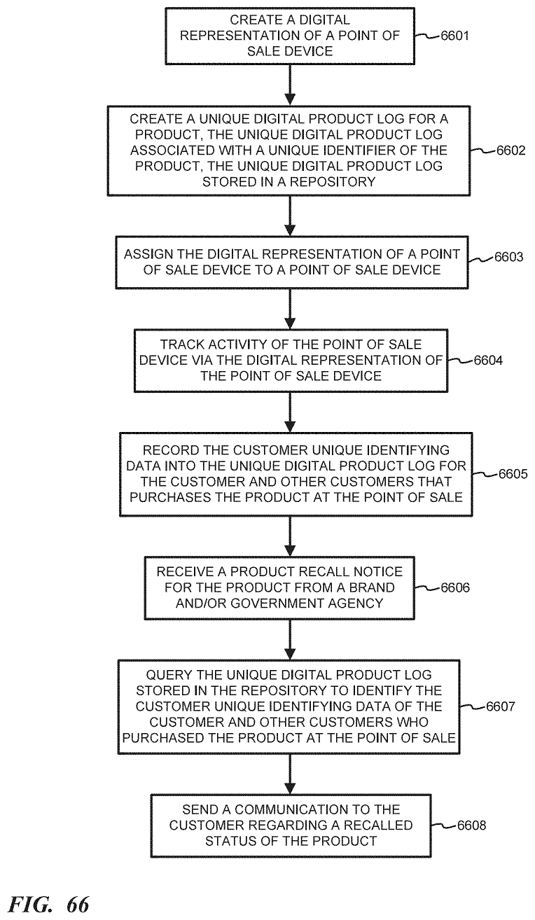

FIG. 66 is a flowchart illustrating a process for notifying a customer of a product recall in an example embodiment. In step 6601, a digital representation of a point of sale device is created. In step 6602, a unique digital product log for a product is created. The unique digital product log is associated with a unique identifier of the product. The unique digital product log is stored in a repository. In step 6603, the digital representation of a point of sale device is assigned to a point of sale device. In step 6604, activity of the point of sale device is tracked via the digital representation of the point of sale device. In step 6605, the customer unique identifying data is recorded into the unique digital product log for the customer and other customers that purchases the product at the point of sale. The customer unique identifying data may be a customer unique identifier.

In step 6606, a product recall notice for the product is received from a brand and/or government agency. In step 6607, the unique digital product log that is stored in the repository is queried to identify the customer unique identifying data of the customer and other customers who purchased the product at the point of sale. In step 6608, a communication is sent to the customer regarding a recalled status of the product.

The process for notifying a customer of a product recall may further include translating data from a native point-of-sale language to a standard system language format.

The process for notifying a customer of a product recall may further include sending product recall information and the customer unique identifier of the customer who purchased the product to a payment processor, and matching customer unique identifier to customer contact information at the payment processor. Sending a communication to the customer regarding the recalled status of the product may be completed by the payment processor.

In another embodiment, a product recall system comprises a digital replica of a point of sale device configured to receive a product unique identifier, and receive customer unique identifying data; an adapter configured to facilitate communication between incompatible entities; a repository configured to store the product unique identifier, and store the customer unique identifying data associated with a purchase of the product; a query module configured to execute a query of the repository to identify customer unique identifying data for the customers who purchased the recalled product; and a communication module configured to receive a communication regarding a product recall, and send communications regarding a product recall.

The repository in the product recall system may be a blockchain repository.

The product recall system may further comprise a second repository.

The product recall system may further comprise an artificial intelligence component configured to direct data to the correct locations.

In another embodiment, a method for stopping a purchase of a product that has been recalled comprises receiving into a system a recall notice; receiving into a point of sale a unique identifier of a recalled product; receiving into the point of sale a communication regarding a recalled status of the product; and stopping the point of sale device from completing a sale transaction for the recalled product.

In another embodiment, a system for alerting a merchant about a defective product in merchant inventory comprises a merchant repository configured to store data regarding products in the merchant inventory; a point of sale device configured to receive product unique identifier data, and receive customer unique identifying data; a blockchain repository configured to store a product unique identifier along with a customer unique identifier for customers who purchased a product associated with the product unique identifier; a querying module configured to execute a query of the blockchain repository to identify customers who purchased a defective product, and execute a query of a merchant repository configured to store merchant inventory data to identify recalled products still in inventory; and a communication module configured to receive a product recall notice, and send a communication to a merchant alerting the merchant of recalled product in the merchant's inventory and recalled product that was sold to merchant customers.

In another embodiment, a method for sending a recall notice to a customer comprises configuring a system repository to receive and store a product log; entering a product unique identifier of a product in order to name the product log; receiving the product unique identifier from a point of sale device; receiving unique identifying data of a customer who is purchasing the product from the point of sale device; writing the unique identifying data of the customer who purchased the product into the product log; receiving a notice recalling the product into the system; querying the system repository to identify the product log and the customer who purchased the product; and sending a product recall notice to the customer. The system repository may be a blockchain repository. An entity sending the product recall notice to the customer may be a payment processor.

The method for sending a recall notice to a customer may further comprise sending to the payment processor a product recall notice with the unique identifying data of the customer who purchased the product. The system repository may be a blockchain repository.

The method for sending a recall notice to a customer may further comprise refunding a purchase price of a recalled product to the customer via a transaction account.

In another embodiment, a system for sending a recall notification for a product to a customer who purchased the product comprises a product log identified by a unique product identifier, the product log configured to receive customer unique identifying data of customers who purchase the product; a repository configured to receive and store the product log; a product with a unique identifier attached to the product; a customer transaction account configured to track payments made by the customer; a payment instrument configured to facilitate payments made from customer transaction account; a point of sale device configured to receive unique product identifier data from the product, and receive customer unique identifying data from the payment instrument; a digital representation of the point of sale device configured within the system to facilitate tracking activity of the point of sale device; a system communication module configured to receive a product recall notification into the system, and send the product recall notification to a payment processor; a system query module configured to execute a query of the of the repository to identify the customer unique identifying data of the customer who purchased the product; a payment processor repository configured to receive customer transaction account information, and store the customer's personal identifying information together with customer unique identifying data associated with the customer account; a payment processor query module configured to execute a query of the payment processor repository to identify personal identifying information associated with the customer unique identifying data; and a payment processor communication module configured to receive the product recall notification and customer unique identifying data of the customer who purchased the recalled product from the system communication module, and send the product recall notification to the customer.

In another embodiment, a method for receiving a recall notice comprises setting up a transaction account with a payment processor; receiving a payment instrument from the payment processor associated with the transaction account; purchasing a product via a point of sale device with the payment instrument; and receiving from the payment processor a notification that the product has been recalled.

In another embodiment, a method for tracking a product for a potential recall comprises entering a product unique identifier associated with a product into a server; receiving into inventory the product with the product unique identifier attached thereto; configuring the server to send customer unique identifying data into a unique product log associated with the product unique identifier; configuring a point of sale device to receive product unique identifier data; configuring the point of sale device to receive the customer unique identifying data from a payment instrument; receiving approval from a payment processor that the customer is authorized to make payment with the payment instrument; selling the product via a point of sale device to the customer using the payment instrument attached to a transaction account; sending customer unique identifying data to the unique product log; and querying the product log to identify customers who purchased the product.

In another embodiment, a method for facilitating a recall of a product comprises setting up a transaction account for a customer; sending a payment instrument to the customer; approving a payment for the purchase of the product by the customer; receiving a communication that the product has been recalled along with unique identifying data of the customer who purchased the recalled product; matching the customer unique identifying data with customer personal identifying information; and sending a communication to the customer with information about the recalled product.

In another embodiment, a system for writing personal preference information and/or allergy information to a customer's purchase receipt comprises a repository configured to receive personal allergy information and/or personal preference information into a customer account stored in the repository, and product ingredients and/or other product information into a product log stored in the repository; a query module configured to execute a query on the repository to identify matches of between customer allergy information and/or personal preference information stored in the customer account and product ingredients and/or other product information stored in the product log; a point of sale device configured to receive customer unique identifying data during a purchase, receive product unique identifier data, and write information to a purchase receipt. The repository may be a blockchain repository.

The system for writing personal preference information and/or allergy information to a customer's purchase receipt may further comprise a communication module configured to send a communication regarding personal preference information and/or allergy information on products the customer purchased to a customer device.

In another embodiment, a method for writing personal preference information and/or allergy information to a customer's purchase receipt comprises receiving personal allergy information and/or personal preference information into a customer account stored in a repository; receiving product ingredients and/or other product information into a product log stored in the repository; receiving into a point of sale device customer unique identifying data during a purchase at the point of sale device; receiving into the point of sale device product unique identifier data during the purchase at the point of sale device; executing a query on the repository to identify matches of between customer allergy information and/or personal preference information stored in the customer account and product ingredients and/or other product information stored in the product log; and writing personal preference information and/or allergy information to the customer's purchase receipt.

The method for writing personal preference information and/or allergy information to a customer's purchase receipt may further comprise sending a communication regarding personal preference information and/or allergy information on products the customer purchased to a customer device. The repository may be a blockchain repository.

In another embodiment, a system for notifying a customer of a recall of a product comprises a point of sale device; a digital representation of the point of sale device configured to facilitate tracking the activity of a point of sale device; a unique digital product log for the product, the unique digital product log associated with a single unique identifier of the product, the unique digital product log configured to receive customer unique identifying data of the customer who purchases the product at the point of sale device, the unique digital product log stored on a repository configured to store the unique digital product log; a querying module configured to execute a query of the unique digital product log stored in the repository to identify the customer unique identifying data of the customer and every customer who purchased the product at the point of sale; a communication module configured to receive a product recall notice from a brand and/or government agency, and to send a communication regarding the recalled status of the product.

The system for notifying a customer of a recall of a product may further comprise a payment processor repository configured to store customer information; a payment processor query module configured to execute a query on the payment processor repository to identify customer contact information associated with customer unique identifying information of the customer who purchased the product; a payment processor communication module configured to receive recall information of the product being recalled, comprising: at least the name of product being recalled, and the customer unique identifying information of the customer who purchased the product, and to send recall information for the product to the customer who purchased the product. The repository may be a blockchain repository.

In another embodiment, a method for notifying a customer of a product recall without disclosing customer's name or contact information comprises receiving into a point of sale device a product unique identifier; receiving into a point of sale from a payment instrument a customer unique identifier; sending the customer unique identifier to a product log identified by the product unique identifier which stores the customer unique identifier of the customer who purchased the product at the point of sale device; receiving a product recall notice for the product with the product unique identifier; querying the product log identified by the product unique identifier to identify the customer unique identifier of the customer who purchased the product; communicating with a payment processor associated with the payment instrument the product name and the customer unique identifier of the customer who purchased the product; querying a payment processor database to identify the customer's personal identifying information matched to the customer unique identifier; and communicating to the customer a notification that the product purchased by the customer has been recalled.

The method for notifying a customer of a product recall without disclosing customer's name or contact information may further comprise storing the product log on a blockchain repository.

In another embodiment, a system for writing data from a point of sale to a blockchain repository comprises a point of sale device configured to receive product data and customer data to generate a transaction log; a data transfer adapter configured to facilitate the transfer of transaction logs; a server configured to apply the appropriate data transfer adapter for transferring transaction logs based on the point of sale type; an order adapter configured to automate complex data normalization so that multiple sources of data are converted into a standard system language format; a data processing adapter configured translate standard system language format into a repository native language; a processing engine configured to apply the appropriate data processing adapter to write to the selected blockchain; and a blockchain repository configured to receive data from the system.

In another embodiment, a method for writing data from a point of sale to a blockchain repository comprises generating a transaction log at a point of sale device that contains product purchase data; transferring the transaction log to a server configured to translate the transaction log; translating the transaction log to a standard system language format file; selecting a portion of the standard system language formal file to write to the blockchain repository; translating the portion of the standard system language format file into the blockchain repository language file; transferring the blockchain repository language file to a blockchain repository node; writing the blockchain repository language format file to the blockchain repository. In some embodiments, the portion includes all portions of the standard system language formal file.

The method for writing data from a point of sale to a blockchain repository may further comprise writing the standard system language format file to a non-blockchain repository.

The method for writing data from a point of sale to a blockchain repository may further comprise selecting a second portion of the standard system language formal file to write to a second blockchain repository; translating the second portion of the standard system language format file into the second blockchain repository language file; and transferring the second blockchain repository language file to a second blockchain repository node.

The method for writing data from a point of sale to a blockchain repository may further comprise selecting a third portion of the standard system language formal file to write to a third blockchain repository; translating the third portion of the standard system language format file into the third blockchain repository language file; and transferring the third blockchain repository language file to a third blockchain repository node

The method for writing data from a point of sale to a blockchain repository may further comprise executing a query across one or more repositories to identify information about consumers and the products they purchased.

In another embodiment, a method for tracking purchases by a customer from multiple merchants comprises signing up a customer for a system account where the customer agrees to have purchases tracked; configuring a digital wallet for the customer; receiving data from a first purchase at a first point of sale at a first merchant; sending first transaction log of the first purchase to cloud system server; translating the first transaction log into standard system language format; sending data from the first purchase to the digital wallet stored in a repository; receiving data from a second purchase at a second point of sale at a second merchant; sending second transaction log from the second purchase to cloud system server; translating the second transaction log into standard system language format; sending purchase data from the second purchase to the digital wallet stored in the repository; and querying the digital wallet stored in the repository to identify purchases made by the customer.

The method for tracking purchases by a customer from multiple merchants may further comprise receiving data from a third purchase at a third point of sale at a second merchant; sending third transaction log of third purchase to cloud system server; translating the third transaction log into standard system language format; and sending purchase data from the third purchase to the digital wallet stored in the repository.

The method for tracking purchases by a customer from multiple merchants may further comprise sending a reward to the customer when a certain level of purchases by the customer is reach.

The method for tracking purchases by a customer from multiple merchants may further comprise receiving into the customer digital wallet store credit that can be spent by the customer at multiple merchants.

In another embodiment, a system for tracking purchases by a customer from multiple merchants comprises a digital wallet assigned to a customer and configured to store data representing purchases made by the customer; a first point of sale device configured to receive product and payment data stationed at a first merchant; a second point of sale device configured to receive product and payment data stationed at a second merchant; digital representations of the point of sale devices configured to facilitate tracking the activity of the point of sale devices and assigned to the point of sale devices; a repository configured to store the digital wallet and receive purchase data into the digital wallet; a query module configured to execute a query of the digital wallet to identify purchases made by the customer.

In another embodiment, a method for offering incentives to a customer based on multiple criteria comprises generating a digital wallet for the customer that contains customer profile data; generating a digital representation of a point of sale device; assigning the digital representation of a point of sale device to a point of sale device to facilitate tracking the activity of the point of sale device; tracking the transaction log of a purchase made by a customer at the point of sale device; sensing at least two of criteria of time criteria, date criteria, location criteria, customer purchases criteria, and customer profile criteria; receiving time criteria and date criteria into the system; receiving customer location criteria into the system; querying the digital wallet of the customer to identify customer criteria and customer purchases criteria; creating a digital incentive for the customer based on at least two criteria; and sending the incentive to the digital wallet of the customer. The incentive may be for use at a location other than location of last known purchase made by customer.

The method for offering incentives to a customer based on multiple criteria may further comprise creating a digital incentive for the customer based on at least three of the criteria.

The method for offering incentives to a customer based on multiple criteria may further comprise creating a digital incentive for the customer based on at least four of the criteria.

The method for offering incentives to a customer based on multiple criteria may further comprise creating a digital incentive for the customer based all criteria available.

In another embodiment, a system for offering incentives to a customer based on multiple criteria comprises a digital wallet assigned to a customer and configured to store data representing purchases made by the customer and to receive incentives sent to the customer; a point of sale device configured to receive product and payment data; a digital representation of a point of sale device configured to facilitate tracking the activity of the point of sale device; a transaction log of a purchase made by the customer at the point of sale device; a time keeping device configured to provide time and date data; a location sensor configured to sense the location of a customer; a query module configured to execute a query of the digital wallet of the customer to identify past purchases of the customer; an engine configured to create incentives based on any two of the following criteria, time criteria, date criteria, location criteria, past behavior of the customer criteria; and a communication module configured to send the incentives to the digital wallet of customer.

In the system for offering incentives to a customer based on multiple criteria the engine may be an artificial intelligence engine. The artificial intelligence engine may be programed to learn preferences of the customer and create incentives based on those preferences. The incentives created may be for a location other than the location of the point of sale device.

In another embodiment, a method for machine to machine tracking of scan-based offers comprises writing a scan-based offer where a brand agrees pay a retailer for meeting a product sales goal; generating a digital product log for the product that resides in a blockchain repository; generating a digital representation of a point of sale device to facilitate tracking the activity of a point of sale device; assigning the digital representation of a point of sale device to the point of sale device; generating a transaction log at the point of sale device; translating the transaction log into a standard system language format; selecting product identifier data for the product from the transaction log; translating product identifier data to be compatible with the blockchain repository; writing the product identifier data to the digital product log that resides in the blockchain repository; and querying the digital product log in the blockchain repository to identify if the sale goal has been met. There may be more than one digital representation of the point of sale assigned to more than one point of sale device. The scan-based offer may be written as a smart contract for the blockchain repository.

The method for machine to machine tracking of scan-based offers may further comprise settling the smart contract automatically when the sales goal is met. The smart contract may be automatically settled by sending a digital form of payment to a transaction account of the retailer.

In another embodiment, a system for machine-to-machine tracking of scan-based offers comprises a scan-based offer in which a brand agrees pay a retailer for meeting a product sales goal; a digital product log for the product that resides in a blockchain repository; a digital representation of a point of sale device configured to facilitate tracking the activity of a point of sale device; a point of sale device configured to receive product and payment data; a data transfer adapter configured to facilitate the transfer of transaction logs; a server configured to apply the appropriate data transfer adapter for transferring transaction logs based on the point of sale type; an order adapter configured to automate complex data normalization so that multiple sources of data are converted into a standard system language format and configured to select the appropriate product data to write to the digital product log.; a data processing adapter configured translate standard system language format into a repository native language; a processing engine configured to apply the appropriate data processing adapter to write selected blockchain; a blockchain repository configured to receive data into the digital product log from the system; and a query module configured to execute a query of the digital product log in the blockchain repository to identify if the sale goal has been met.

In the system for machine-to-machine tracking of scan-based offers, there may be more than one digital representation of the point of sale assigned to more than one point of sale device. The scan-based offer may be written as a smart contract for the blockchain repository. The smart contract may be configured to settle automatically when the sales goal is met. The smart contract may be configured to automatically settled by sending a digital form of payment to a transaction account of the retailer.

In another embodiment, a system for dynamic pricing for restricted items allows CPG brands to offer direct consumer incentives (e.g., rebates) for alcohol and other articles restricted by laws that differ by area and other factors. The system knows all the relevant information and only incentivizes individuals in a manner that is legally acceptable, such as based on the area, age, and other factors. The system can instantly incentivize (e.g., rebate) a consumer, thereby clearing with the brand and the retailers in a matter of seconds. The system allows for personalized pricing of alcohol and other restricted goods.

In another embodiment, a method for offering an incentive for a restricted item to a customer based on age, location, and other criteria comprises generating a digital wallet for the customer that contains customer specifics comprises sensing location of the customer; querying the digital wallet and law repository to identify age, laws pertaining to the restricted item in the location of the customer, and other pertinent criteria; creating a digital incentive for the customer based on age, laws in the location of the customer and other criteria; and sending the incentive to the digital wallet of the customer.

In another embodiment, a system for offering incentives for a restricted item to a customer based on age, location and other criteria comprises a digital wallet assigned to a customer and configured to receive incentives sent to the customer; a digital wallet repository configured to store digital wallets; a time keeping device configured to provide time and date data; a location sensor configured to sense the location of a customer; a law repository configured to store laws pertaining to the restricted item searchable by location; a query module configured to execute a query of the digital wallet of the customer to identify age and other criteria, and a query of the law repository to identify laws pertaining to the restricted item in the location of the customer; an engine configured to create incentives based on age, location and other criteria; and a communication module configured to send the incentives to the digital wallet of customer.

The system for offering incentives for a restricted item to a customer based on age, location and other criteria wherein the engine is an artificial intelligence engine. The artificial intelligence engine may be programed to learn preferences of the customer and create incentives based on those preferences as well as other criteria.

Millennials, like most generations, enjoy being rewarded for their actions. Through gamification, brands may offer discount experiences based on milestones and rewards. Consumers are surprised and delighted by brands that they previously thought to be boring and old. One object of the invention is to provide a permission blockchain system and artificial intelligence gamification of incentives.

In another embodiment, a method for offering an incentive to a customer based on customer performance of tasks comprises generating a digital wallet assigned to a customer and configured to store data representing purchases made by the customer, tasks completed by the customer and to receive incentives sent to the customer; sending an offer via the communication module to the customer to receive an incentive based on the customer performance of tasks; generating a digital representation of a point of sale device configured to facilitate tracking the activity of a point of sale device; generating a purchase log of purchases made by the customer at point of sale devices, writing the purchase log to the digital wallet; receiving time and date data from a time keeping device; receiving location information of the customer from a sensor configured to sense the location of a customer; receiving customer content viewing information; querying the digital wallet of the customer to identify if the customer has performed tasks required to receive the incentive; and sending via the communication module an incentive to the digital wallet of the customer.

The method for offering an incentive to a customer based on customer performance of tasks wherein the offer is generated via an artificial intelligence engine. The artificial intelligence engine may be programed to learn preferences of the customer and create performance-based incentives based on those preferences.

In another embodiment, a system for offering an incentive to a customer based on customer performance of tasks comprises an offer to a customer to receive an incentive-based customer performance of tasks; a digital wallet assigned to the customer and configured to store data representing purchases made by the customer, tasks completed by the customer and to receive incentives sent to the customer; a point of sale device configured to receive product and payment data; a digital representation of a point of sale device configured to facilitate tracking the activity of the point of sale device; a purchase log of a purchases made by the customer at point of sale devices store in the digital wallet; a time keeping device configured to provide time and date data; a location sensor configured to sense the location of a customer; a query module configured to execute a query of the digital wallet of the customer to identify if the customer has performed tasks required to receive the incentive; and a communication module configured to send offers and incentives to the digital wallet of the customer

The system for offering an incentive to a customer based on customer performance of tasks further comprising an artificial intelligence engine. The artificial intelligence engine may be programed to learn preferences of the customer and create performance-based incentives based on those preferences.

One embodiment provides enhance scalability and speed of a ledger or database system through sharding. The system of sharding allows each player in the blockchain to have their own blockchain. Embodiments provide centralized connection information where horizontal database sharding is implemented. When any of the business objects (e.g., hundreds of business objects) are persisted, the system asks the database connection manager where it should be persisted. The connection manager determines the type of object (Order, for example) and the retail hierarchy (e.g., client, program, merchant, and store) where the order is to be persisted and returns what order database shard the order should use. Given that all logic is isolated from the developer, modifications to the algorithm are easy and safe to implement.

The OBM plug-in works similarly and seamlessly adds an additional save operation to the blockchain. It saves to the database, and with the plug-in, the Object would know its destination blockchain-shard based on the transaction being brand, retailer, or partner. In addition, it will know under which client, program, merchant, and site the transaction is taking place and know which shard it should use when saving to the blockchain.

In another embodiment, a system for sharding in a permissioned blockchain repository where multiple parties are each assigned their own blockchain shard comprises a blockchain repository stored on two or more nodes and configured to receive data via blocks each block with a beginning hash and an ending hash; nodes configured to store the blockchain repository and validate blocks within the blockchain repository; a digital representation of a first party; and a digital representation of a second party. A repository connection manager may be configured to assign a shard location to digital representations whenever they are persisted.

In another embodiment, a method of offering credit at a point of sale comprises receiving from a customer account signup information for a credit account including at least the customer's name; recording customer information to a repository; assigning a digital representation of a point of sale device to a point of sale device that is configured to interact with the customer; executing a query of a repository to identify if a credit account of the customer is valid, and if the account has available credit to pay for a transaction; recording a credit receivable to a repository; sending a payment to a merchant for the transaction; and recording payment information to a repository.

In another embodiment, a method for offering credit to a customer at a point of sale comprises setting up an account for a customer; recording customer account to a repository; receiving a request for payment from a device of the customer; assigning to the device of the customer a digital representation of the device of the customer; executing a query of the customer account to identify if a credit account of the customer is valid, and if the credit account has available credit to pay for a transaction; sending to the device of the customer a digital signature; assigning to a point of sale a digital representation of the point of sale; receiving from the point of sale the digital signature; and recording a credit receivable to the repository. The repository may be a blockchain repository

The method for offering credit to a customer at a point of sale may further comprise verifying the location of the customer device.

The method for offering credit to a customer at a point of sale may further comprise verifying that the customer is operating the customer device.

In another embodiment, a method for providing credit to a customer comprises creating a customer digital wallet; creating a merchant digital wallet; creating a funding account; creating digitized tokens that represent funds held in a separate account; sending digitized tokens to the funding account; creating a digital representation of a point of sale device configured to facilitate tracking of a point of sale device; receiving into the point of sale device product information; receiving into the point of sale customer digital wallet information; receiving a request from the point of sale device to authorize a payment; sending an approval message to the point of sale device; sending digitized tokens to the digital wallet of the merchant from the funding account; sending request for payment to the digital wallet of the customer; sending digital wallet information to a repository; and sending funding account information to the repository

In another embodiment, a system for providing credit to a customer comprises a digital wallet configured to receive personal information of the customer and transaction information of the customer; a digital wallet configured to receive information of a merchant; a digital representation of a point of sale device configured to allow interaction with a point of sale device; digitized tokens configured to represent funds held in a separate account; a credit funding account configured to receive and disperse digitized tokens; a repository configured to receive and store account information; a digital wallet app configured to allow inputs from a customer and to facilitate transaction payments. The repository may be a blockchain repository.

Although this disclosure specifically described best modes and preferred methods and use of the invention, it should be understood that many changes in the specific methods and modes described and shown in the figures may clearly be made without departing from the true scope of the invention. Also, it is anticipated that all drawings and paragraphs or parts within this specification can be combined with any other drawing and/or paragraph to make the intended invention. While various exemplary embodiments of the disclosed system and method have been described above it should be understood that they have been presented for purposes of example only, not limitations. It is not exhaustive and does not limit the disclosure to the precise form disclosed. Modifications and variations are possible in light of the above teachings or may be acquired from practicing of the disclosure, without departing from the breadth or scope.

1. A method of consumer authorization, comprising:
creating a digital representation of a point of sale device;
assigning the digital representation of the point of sale device to a device in order to track activity of a consumer;
writing to a repository associated with the digital representation of the point of sale device consumer unique identifying data;
writing, to the repository associated with the digital representation of the point of sale device, unique product identifying data associated with a pharmaceutical received by the consumer;
writing the unique product identifying data to a repository;
sensing proximity of the device;
receiving, from the digital representation of the point of sale device, consumer unique identifying data;
sensing a health issue of the consumer;
writing the proximity data and health issue of the consumer data to the repository;
analyzing via an artificial intelligence component the proximity data and the health issue of the consumer data in the repository;
generating a response to the analysis of the proximity data and the health issue of the consumer data;
retrieving, from the repository, the consumer unique identifying data, the product unique identifying data that is associated with a pharmaceutical received by the consumer, and artificial intelligence analysis data; and
authorizing services for the consumer dependent on the data retrieved from the repository.

2. The method of claim 1, wherein the device is associated with a consumer payment instrument.

3. The method of claim 1, wherein the device is implanted in the consumer.

4. The method of claim 1, wherein the repository is a blockchain repository.

5. The method of claim 1, wherein the data regarding the digital representation of the point of sale device, the device data, product unique identifying data, device proximity data, health issue data, artificial intelligence analysis data are stored in a log associated with the consumer.

6. The method of claim 1, further comprising:
sending a pharmaceutical adverse reaction notice to the consumer.

7. The method of claim 1, further comprising:
sending a health report to the consumer.

8. The method of claim 1, further comprising:
translating data from a sensor to a standard system language format.

9. A consumer identity safety system, comprising:
a digital replica of a point of sale device configured to
receive a pharmaceutical product unique identifier, and
receive consumer unique identifying data;
a recognition interface configured to identify a consumer;
sensors configured to sense health issues of a consumer;
an adapter configured to facilitate communication between sensors and the system;
a repository configured to
receive and store the pharmaceutical product unique identifier, and
receive and store the consumer unique identifying data and pharmaceutical product unique identifier associated with consumer receipt of a pharmaceutical product; and
receive and store consumer health issue data; and
receive and store consumer proximity data;
a query module configured to
execute a query of the repository to identify consumer unique identifying data of the consumers who received the pharmaceutical product along with consumer health issue data; and
a communication module configured to
receive a communication regarding a consumer health issue; and
send communications regarding a consumer health issue.

10. The system of claim 9, wherein the repository is a blockchain repository.

11. The system of claim 10, further comprising:
a second repository.

12. The system of claim 11, further comprising:
an artificial intelligence component configured to analyze consumer health issue data and direct data to the correct locations.

13. A method for authorizing access to a space, comprising:
creating a digital replica of a point of sale device;
assigning the digital replica to a point of sale device;
receiving consumer pharmaceutical receipt data into a repository;
receiving point of sale proximity data into repository;
recognizing a consumer with the point of sale device at a check point
executing a query of the repository to match consumer with consumer pharmaceutical receipt data;
sending to a device a communication regarding a health issue of the consumer; and
authorizing access to a space dependent on said communication.

14. The method of claim 13, further comprising:
tracking point of sale device activity via the digital replica of the point of sale device.

15. The method of claim 13, further comprising:
receiving from the digital replica the unique identifier of a pharmaceutical product known to cause an adverse reaction.

16. (canceled)

17. The method for claim 13, further comprising:
causing to be displayed on the point of sale device a notice about a known adverse reaction to a pharmaceutical product.

18. (canceled)

19. (canceled)

20. (canceled)

21. (canceled)

22. (canceled)

23. (canceled)

24. A system for access authorization of a consumer, comprising:
a digital representation of a consumer;
a recognition interface configured to recognize the consumer;
a log associated with the digital representation of the consumer configured to accept a pharmaceutical product unique identifier;
a virtual space configured to represent a physical environment;
a query module configured to query the log to determine if the consumer has received the pharmaceutical; and
a virtual machine configured to authorize access to a physical space associated with the virtual space.

25. The system of claim 24, further comprising:
sensors configured to detect consumer health issues.

26. The system of claim 25, wherein the log is also configured to receive consumer health issue data.

27. The system of claim 25, further comprising:
an implant configured to be implanted at least partially under the skin of the consumer; and
wherein the recognition interface is configured to scan the implant for a unique identifier.

28. The system of claim 25, further comprising:
an artificial intelligence component configured to analyze data received into the system from the sensors and generate a response regarding the data analysis.
